(19) United States
(12) Patent Application Publication
(10) Pub. No.: US 2004/0072228 A1
(43) Pub. Date: Apr. 15, 2004

(54) Card11 NFkB activating polypeptides, nucleic acids, inbred and transgenic animals, and methods of use thereof

(75) Inventors: Richard J. Glynne, La Jolla, CA (US); Jesse Eunsuk Jun, Palmerston (AU); Christopher Carl Goodnow, Ainslie (AU)

Correspondence Address: FOLEY & LARDNER, P.O. BOX 80278, SAN DIEGO, CA, 92138-0278, US

(73) Assignee: Phenomix Corporation

(21) Appl. No.: 10/632,696

(22) Filed: Aug. 1, 2003

Related U.S. Application Data
(60) Provisional application No. 60/401,078, filed on Aug. 2, 2002.
(60) Provisional application No. 60/422,614, filed on Oct. 29, 2002.

(51) Int. Cl. C12Q 1/68; C07H 21/04; C12N 9/12; C12P 21/02; C12N 5/06

(52) U.S. Cl. 435/6; 435/69.1; 435/194; 435/320.1; 435/325; 536/23.2

(57) ABSTRACT

The invention provides novel caspase recruitment domain 11 (CARD11), also known as CARMA-1, polypeptides, nucleic acids encoding them and methods for making and using them. In one aspect, the polypeptides of the invention have NFkB activating activity. The invention also provides non-human transgenic animals, e.g., mice, comprising the CARD11 nucleic acids of the invention. The invention also provides pharmaceutical compositions comprising a nucleic acid or polypeptide of the invention. Administration of a pharmaceutical composition of the invention to a subject is used to generate a toleragenic immunological environment in the subject. This can be used to tolerize the subject to an antigen. The invention also provides inbred mouse strains homozygous for a non-wild type CARD11 allele. This genotype results in mice having a phenotype comprising dermatitis, B cell defects and T cell defects.

### FIELD OF THE INVENTION

This invention relates to molecular and cellular biology and medicine. In particular, the invention provides novel caspase recruitment domain 11 (CARD11), also known as CARMA-1, polypeptides, nucleic acids encoding them and methods for making and using them, for example, to produce non-human transgenic animals.

### BACKGROUND OF THE INVENTION

The caspase recruitment domain (CARD) polypeptide can function as a protein-binding module to mediate the assembly of CARD-containing proteins into apoptosis and NF-kappaB signaling complexes. It has been reported that CARD protein 11 (CARD11), also known as CARMA-1, and CARD protein 14 (CARD14) maybe members of a membrane-associated guanylate kinase (MAGUK) family, a class of proteins that functions as molecular scaffolds for the assembly of multiprotein complexes at specialized regions of the plasma membrane. CARD11 and CARD14 have been reported to have homologous structures consisting of an N-terminal CARD domain, a central coiled-coil domain, and a C-terminal tripartite domain comprised of a PDZ domain, an Src homology 3 domain, and a GUK domain with homology to guanylate kinase. The CARD domains of both CARD11 and CARD14 may associate with the CARD domain of BCL10, a signaling protein that activates NF-kappaB through the I-kappa-B kinase complex in response to upstream stimuli. It was reported that when expressed in cells, CARD11 and CARD14 activate NF-kappa B and induce the phosphorylation of BCL11. These findings suggest that CARD11 and CARD14 are MAGUK family members that function as upstream activators of BCL10 and NF-kappaB signaling. See, e.g., Bertin (2001) J. Biol. Chem. 276(15):11877-11882; WO 01/40468.

### SUMMARY OF THE INVENTION

The present invention provides novel caspase recruitment domain 11 (CARD11), also known as CARMA-1, polypeptides, nucleic acids encoding them and methods for making and using them. In one aspect, the polypeptides of the invention have NFKB activating activity. The invention also provides non-human transgenic animals, e.g., mice, comprising the CARD11 nucleic acids of the invention. The invention also provides pharmaceutical compositions comprising a nucleic acid or polypeptide of the invention. Administration of a pharmaceutical composition of the invention to a subject is used to generate a toleragenic immunological environment in the subject. This can be used to tolerize the subject to an antigen. The invention also provides inbred mouse strains homozygous for a non-wild type CARD11 allele. This genotype results in mice having a phenotype comprising dermatitis, B cell defects and T cell defects.

In one aspect, the invention provides an isolated or recombinant nucleic acid comprising a nucleic acid sequence having at least 90% sequence identity to SEQ ID NO:1, wherein the nucleic acid encodes at least one polypeptide having an NFkB activating activity. In alternative embodiments, the NFkB activating activity comprises mediating the assembly of CARD-containing proteins into apoptosis signaling complexes, mediating the assembly of CARD-containing proteins into NF-kappaB signaling complexes, or being a molecular scaffold for the assembly of a multi-molecular complex. In further alternative embodiments, the NFkB activating activity comprises recruitment of a signaling protein into a multi-molecular complex, wherein the signaling protein comprises a Bcl10, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

The invention provides isolated or recombinant nucleic acids comprising a nucleic acid sequence having at least 90% sequence identity to SEQ ID NO:1 over a region of at least about 100 residues, wherein the nucleic acids encode at least one polypeptide having an NFkB activating activity. The sequence identities can be determined by analysis with a sequence comparison algorithm or by a visual inspection.

In alternative aspects, the isolated or recombinant nucleic acids comprise a nucleic acid sequence having at least 90%, 91%, 92%, 93%, 94%, 95%, 96%, 97%, 98%, 99% or more sequence identity to SEQ ID NO:1 over a region of at least about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues. In one aspect, the isolated or recombinant nucleic acid has a sequence as set forth in SEQ ID NO:1.

The invention provides isolated or recombinant nucleic acids comprising a nucleic acid sequence having at least 90% sequence identity to SEQ ID NO:3 over a region of at least about 100 residues. In one aspect, the nucleic acid encodes at least one polypeptide having an NFkB activating activity. The sequence identities can be determined by analysis with a sequence comparison algorithm or by a visual inspection.

In alternative aspects, the isolated or recombinant nucleic acids comprise a nucleic acid sequence having at least 90%, 91%, 92%, 93%, 94%, 95%, 96%, 97%, 98%, 99% or more sequence identity to SEQ ID NO:3 over a region of at least about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues. In one aspect, the isolated or recombinant nucleic acid has a sequence as set forth in SEQ ID NO:3.

The invention provides isolated or recombinant nucleic acids comprising a nucleic acid sequence having at least 90% sequence identity to SEQ ID NO:4 over a region of at least about 100 residues. The sequence identities can be determined by analysis with a sequence comparison algorithm or by a visual inspection.

In alternative aspects, the isolated or recombinant nucleic acids comprise a nucleic acid sequence having at least 90%, 91%, 92%, 93%, 94%, 95%, 96%, 97%, 98%, 99% or more sequence identity to SEQ ID NO:4 over a region of at least about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues. In one aspect, the isolated or recombinant nucleic acid has a sequence as set forth in SEQ ID NO:4.

The sequence comparison algorithm can be a BLAST version algorithm. In one aspect, the BLAST nucleotide parameters comprise word size=11, expect=10, filter low complexity with DUST, cost to open gap=5, cost to extend gap=2, penalty for mismatch=−3, reward for match=1, Dropoff (X) for BLAST extensions in bits=20, final X dropoff value for gapped alignment=50, and all other options are set to default.

In one aspect, the NFkB activating activity comprises mediating the assembly of CARD-containing proteins into apoptosis signaling complexes. The NFkB activating activity can comprise mediating the assembly of CARD-containing proteins into NF-kappaB signaling complexes. The NFkB activating activity can comprise being a molecular scaffold for the assembly of a multi-molecular complex. In one aspect, the NFkB activating activity comprises being a molecular scaffold for the assembly of a multiprotein complex.

In one aspect, the NFkB activating activity comprises recruitment of a signaling protein into a multi-molecular complex. The NFkB activating activity can comprise recruitment of a signaling protein into a multi-molecular complex comprising a lipid raft. The signaling protein can comprise a Bc110, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

In one aspect, the polypeptide can have a membrane-associated guanylate kinase domain structure. The polypeptides can have a central coiled-coil domain. The polypeptide can have a C-terminal tripartite domain comprising a PDZ domain, an Src homology 3 domain or a GUK domain.

The invention provides isolated or recombinant nucleic acids, wherein the nucleic acids comprise a sequence that hybridizes under stringent conditions to a nucleic acid sequence as set forth in SEQ ID NO:1, SEQ ID NO:3, or SEQ ID NO:4, or the complement of any thereof, wherein the nucleic acid encodes a polypeptide having an NFkB activating activity. In one aspect, the nucleic acid encodes a polypeptide having an NFkB activating activity. In alternative aspects, the nucleic acid comprises at least about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues in length, or, the full length of the gene or transcript. In one aspect, the stringent conditions comprise a wash step comprising a wash in 0.2×SSC at a temperature of about 65° C. for about 15 minutes.

In further aspects, the invention provides an isolated or recombinant nucleic acid comprising a nucleic acid sequence having at least 90% sequence identity to SEQ ID NO:3; and an isolated or recombinant nucleic acid comprising a nucleic acid sequence having at least 90% sequence identity to SEQ ID NO:4.

In another aspect, the invention provides first nucleic acid molecule for identifying a second nucleic acid molecule, wherein the second nucleic acid molecule encodes a polypeptide with an NFkB activating activity, wherein the first nucleic acid molecule comprises at least 10 consecutive bases of a sequence as set forth in SEQ ID NO:1, SEQ ID NO:3 or SEQ ID NO:4, and further wherein the first nucleic acid molecule identifies the second nucleic acid molecule by binding or hybridization.

The invention provides nucleic acid probes for identifying a nucleic acid encoding a polypeptide with an NFkB activating activity or a CARD11 gene or variant thereof, wherein the probe comprises at least 10 consecutive bases of a sequence as set forth in SEQ ID NO:1, SEQ ID NO:3 or SEQ ID NO:4, wherein the probe identifies the nucleic acid by binding or hybridization. The probe preferably comprises at least 15, 16, 17, 18, 19, 20, 21, 22, 23, 24 or 25 consecutive bases. The invention provides nucleic acid probes for identifying a nucleic acid encoding a polypeptide with an NFkB activating activity or a CARD11 gene or variant thereof, wherein the probe comprises a nucleic acid sequence having at least 90%, 91%, 92%, 93%, 94%, 95%, 96%, 97%, 98%, 99% or more sequence identity to SEQ ID NO:1, SEQ ID NO:3 or SEQ ID NO:4 over a region of at least about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues. The sequence identities can be determined by analysis with a sequence comparison algorithm or by visual inspection. In alternative aspects, the probe comprises an oligonucleotide comprising at least about 10 to 50, about 20 to 60, about 30 to 70, about 40 to 80, or about 60 to 100 consecutive bases of a sequence as set forth in SEQ ID NO:1, SEQ ID NO:3 or SEQ ID NO:4. The probe preferably comprises at least 15, 16, 17, 18, 19, 20, 21, 22, 23, 24 or 25 consecutive bases. In alternative aspects, the probe is about 90, 100, 110, 120, 130, 140, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or more residues in length. In one aspect, the probe comprises a subset of a sequence as set forth in SEQ ID NO:1, SEQ ID NO:3 or SEQ ID NO:4.

The invention provides an amplification primer sequence pair for amplifying a nucleic acid of the invention, e.g., a nucleic acid encoding a polypeptide with an NFkB activating activity. The primer pair can be capable of amplifying a nucleic acid sequence as set forth in SEQ ID NO:1, SEQ ID NO:3 or SEQ ID NO:4. In one aspect, each member of the amplification primer sequence pair comprises an oligonucleotide comprising at least about 10 to 50 consecutive bases of the sequence.

The invention provides methods of amplifying a nucleic acid encoding a polypeptide with an NFkB activating activity or a CARD11 gene or variant thereof, comprising amplification of a template nucleic acid with an amplification primer sequence pair capable of amplifying a nucleic acid sequence as set forth in SEQ ID NO:1, SEQ ID NO:3 or SEQ ID NO:4.

In further aspects, the invention provides an expression cassette, a vector, or a transformed cell comprising a nucleic acid comprising a nucleic acid sequence having at least 90% sequence identity to SEQ ID NO:1, SEQ ID NO:3, or SEQ ID NO:4. The transformed cell may be a bacterial cell, a mammalian cell, a fungal cell, a yeast cell, an insect cell or a plant cell. The invention further provides a cloning vehicle comprising a nucleic acid sequence having at least 90% sequence identity to SEQ ID NO:1, SEQ ID NO:3, or SEQ ID NO:4. The nucleic acid sequence may be contained within a recombinant virus, a plasmid, a phage, a phagemid, a cosmid, a fosmid, a bacteriophage or an artificial chromosome.

The invention provides expression cassettes comprising a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof.

The invention provides vectors comprising a nucleic acid of the invention, e.g., a nucleic acid comprising a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof.

The invention provides vector systems comprising a nucleic acid of the invention, e.g., a nucleic acid having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof, operably linked to transcriptional control elements such that double stranded RNA molecules are produced, wherein the double stranded RNA molecules are complementary to or capable of hybridizing under stringent conditions to a nucleic acid sequence at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. In one aspect, the vector systems and transcriptional control elements can generate intracellular double stranded RNA molecules.

The invention provides cloning vehicles comprising a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. In one aspect, the cloning vehicle comprises a recombinant virus, a plasmid, a phage, a phagemid, a cosmid, a fosmid, a bacteriophage or an artificial chromosome. The virus can comprise an adenovirus vector, a retroviral vector or an adeno-associated viral vector. The cloning vehicle can comprise a bacterial artificial chromosome (BAC), a plasmid, a bacteriophage P1-derived vector (PAC), a yeast artificial chromosome (YAC), a mammalian artificial chromosome (MAC).

The invention provides transformed cells comprising a vector, wherein the vector comprises a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. In alternative aspects, the cell is a bacterial cell, a mammalian cell, a fungal cell, a yeast cell, an insect cell or a plant cell.

In further aspects, the invention provides an antisense oligonucleotide comprising a nucleic acid sequence complementary to or capable of hybridizing under stringent conditions to a nucleic acid sequence with at least 90% sequence identity to SEQ ID NO:1, SEQ ID NO:3, or SEQ ID NO:4; and a method of inhibiting an NFkB activity in a cell with this antisense.

The invention provides antisense oligonucleotides comprising a nucleic acid of the invention, e.g., a nucleic acid sequence complementary to or capable of hybridizing under stringent conditions to a nucleic acid sequence at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. The antisense oligonucleotide can be between about 10 to 50, about 20 to 60, about 30 to 70, about 40 to 80, or about 60 to 100 bases in length.

The invention provides methods of inhibiting an NFkB activity in a cell comprising administering to the cell or expressing in the cell an antisense oligonucleotide comprising a nucleic acid of the invention, e.g., a nucleic acid sequence complementary to or capable of hybridizing under stringent conditions to a nucleic acid sequence at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof.

In further aspects, the invention provides a double stranded RNA oligonucleotide comprising a nucleic acid sequence complementary to or capable of hybridizing under stringent conditions to a nucleic acid sequence with at least 90% sequence identity to SEQ ID NO:1, SEQ ID NO:3, or SEQ ID NO:4. The invention further provides a method of inducing the degradation by RNA interference of a message in a cell comprising administering to the cell or expressing in the cell a double stranded RNA molecule, or a molecule predicted to fold into a double stranded form, or two complementary RNA molecules that are capable of hybridizing to form a double stranded RNA molecule, wherein the double stranded RNA molecule comprises a nucleic acid sequence complementary to or capable of hybridizing under stringent conditions to a nucleic acid sequence at least 90% sequence identity to SEQ ID NO:1, SEQ ID NO:3, or SEQ ID NO:4.

The invention provides double stranded RNA oligonucleotides comprising a nucleic acid of the invention, e.g., a nucleic acid sequence complementary to or capable of hybridizing under stringent conditions to a nucleic acid sequence at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. The double stranded RNA oligonucleotide can be between about 10 to 50, about 20 to 60, about 30 to 70, about 40 to 80, or about 60 to 100 bases in length.

The invention provides methods of inducing the degradation by RNA interference of a message in a cell comprising administering to the cell or expressing in the cell a double stranded RNA molecule, or a molecule predicted to fold into a double stranded form, or two complementary RNA molecules that are capable of hybridizing to form a double stranded RNA molecule, wherein the double stranded RNA molecule comprises a nucleic acid of the invention, e.g., a nucleic acid sequence complementary to or capable of hybridizing under stringent conditions to a nucleic acid sequence at least 90% sequence identity to SEQ ID NO:1 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, or a subsequence or complement thereof.

In one aspect, the sequence comparison algorithm is a BLAST version algorithm, e.g., where the BLAST nucleotide parameters comprise word size=3, expect=10, filter low complexity with SEG, cost to open gap=11, cost to extend gap=1, similarity matrix Blosum62, Dropoff(X) for blast extensions in bits=7, X dropoff value for gapped alignment (in bits)=15, final X dropoff value for gapped alignment=25.

In another aspect, the invention provides an isolated or recombinant polypeptide comprising (i) an amino acid sequence having at least 95% sequence identity to SEQ ID NO:2; or (ii) an amino acid sequence encoded by a nucleic acid comprising a sequence having at least 90% sequence identity to SEQ ID NO:1; wherein the polypeptide has an NFkB activating activity. The NFkB activating activity comprises mediating the assembly of CARD-containing proteins into apoptosis signaling complexes, mediating the assembly of CARD-containing proteins into NF-kappaB signaling complexes, or being a molecular scaffold for the assembly of a multi-molecular complex. Alternatively, the NFkB activating activity comprises recruitment of a signaling protein into a multi-molecular complex. The signaling protein may comprise a Bc110, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

The invention provides isolated or recombinant polypeptides comprising an amino acid sequence having at least 95%, 96%, 97%, 98%, 99% or more sequence identity to SEQ ID NO:2 or SEQ ID NO:5 over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention, e.g., a nucleic acid comprising a sequence: (i) having at least 90% sequence identity to SEQ ID NO:1 over a region of at least about 100 residues, wherein the sequence identities are determined by analysis with a sequence comparison algorithm or by visual inspection, or, (ii) that hybridizes under stringent conditions to a nucleic acid as set forth in SEQ ID NO:1 or a complement thereof. In one aspect, the polypeptide comprises a sequence as set forth in SEQ ID NO:2 or SEQ ID NO:5.

In one aspect, the polypeptide has an NFkB activating activity. The NFkB activating activity can comprise mediating the assembly of CARD-containing proteins into apoptosis signaling complexes. The NFkB activating activity can comprise mediating the assembly of CARD-containing proteins into NF-kappaB signaling complexes. The NFkB activating activity can comprise being a molecular scaffold for the assembly of a multi-molecular complex, or, being a molecular scaffold for the assembly of a multiprotein complex. The NFkB activating activity can comprise recruitment of a signaling protein into a multi-molecular complex. The NFkB activating activity can comprise recruitment of a signaling protein into a multi-molecular complex comprising a lipid raft. The signaling protein can comprise a Bc110, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

In one aspect, the polypeptides of the invention have a membrane-associated guanylate kinase domain structure. In one aspect, the polypeptide of the invention have a central coiled-coil domain. The polypeptides of the invention can have a C-terminal tripartite domain comprising a PDZ domain, a Src homology 3 domain or a GUK domain.

The invention provides protein preparations comprising a polypeptide of the invention, wherein the protein preparation can comprise a liquid, a solid or a gel.

The invention provides heterodimers comprising a polypeptide of the invention. In one aspect, the second domain comprises a polypeptide and the heterodimer is a fusion protein. The second domain can be an epitope or a tag.

The invention provides immobilized polypeptides of the invention. In one aspect, the polypeptides have an NFkB activating activity, wherein the polypeptide comprises a sequence of the invention. In one aspect, the polypeptides comprise antibodies of the invention. The polypeptide can be immobilized on a cell, a metal, a resin, a polymer, a ceramic, a glass, a microelectrode, a graphitic particle, a bead, a gel, a plate or a capillary tube.

The invention provides arrays comprising an immobilized polypeptide of the invention, e.g., an antibody of the invention or a CARD11 polypeptide of the invention. The invention provides arrays comprising an immobilized nucleic acid of the invention.

The invention provides isolated or recombinant antibodies that specifically bind to a polypeptide of the invention. The antibody can be a monoclonal or a polyclonal antibody. The invention provides hybridomas comprising an antibody that specifically binds to a polypeptide of the invention.

The invention provides methods of making an antibody that specifically binds to a polypeptide with NFkB activating activity comprising administering to a non-human animal a nucleic acid of the invention, or a polypeptide of the invention, in an amount and form sufficient to generate a humoral immune response, thereby making an antibody that specifically binds to a polypeptide with NFkB activating activity.

The invention provides methods of producing a recombinant polypeptide comprising the steps of: (a) providing a nucleic acid operably linked to a promoter; wherein the nucleic acid comprises a sequence of the invention; and (b) expressing the nucleic acid of step (a) under conditions that allow expression of the polypeptide, thereby producing a recombinant polypeptide. The methods can further comprise transforming a host cell with the nucleic acid of step (a) followed by expressing the nucleic acid of step (a), thereby producing a recombinant polypeptide in a transformed cell.

According to a further aspect, the invention provides a method for identifying a polypeptide having an NFkB activating activity. This method comprises contacting a polypeptide of the invention, or a fragment thereof, with two or more molecules that multimerize or specifically associate in the presence of an NFkB activating polypeptide; and detecting multimerization or specific association of the molecules; wherein multimerization or specific association of the molecules identifies the polypeptide as having an NFkB activating activity.

According to a further aspect, the invention provides another method for identifying a polypeptide having an NFkB activating activity. This method comprises contacting a polypeptide of the invention, or a fragment thereof, with a construct comprising an NFkB-responsive promoter operably linked to a reporter gene; and detecting the amount of reporter gene product produced; wherein an increase in the amount of reporter gene product identifies the polypeptide as having an NFkB activating activity.

The invention provides methods for identifying a polypeptide having an NFkB activating activity comprising the following steps: (a) providing a polypeptide of the invention or a polypeptide encoded by a nucleic acid of the invention; (b) providing a construct comprising an NFkB-responsive promoter operably linked to a reporter gene; and (c) contacting the polypeptide of step (a) with the construct of step (b) and detecting the amount of reporter gene product produced, wherein an increase in the amount product identifies the polypeptide as having an NFkB activating activity. In one aspect, the NFkB activating activity comprises recruitment of a signaling protein into a multi-molecular complex comprising a lipid raft. The NFkB activating activity can comprise being a molecular scaffold for the assembly of a multiprotein complex. The NFkB activating activity can comprise activation of IKK complex, degradation of p100 or phosphorylation of RelA (p65), c-Rel or RelB. The NFkB activating activity can comprise stimulation of cellular apoptosis, cell proliferation, entry into mitosis or upregulation of NFkB-response polypeptides.

In one aspect, detecting the amount of reporter gene product produced comprises detecting the amount of reporter gene transcript, the amount of polypeptide encoded by the reporter gene, or, where the reporter gene encodes an enzyme, detecting the amount of the enzyme's product or substrate. The reporter gene can encode a CD25, a TNF alpha, an interleukin 1, an interleukin 6, a CD25, a MIP1a, a MIP1b, a Bc1X1, an A1, an IRF4 or a detectable moiety, such as a chemo-luminescent polypeptide, e.g., a luciferase, and the like.

According to a further aspect, the invention provides a method of determining whether a test compound specifically binds to a polypeptide of the invention, or a fragment thereof. This method comprises contacting the polypeptide or fragment thereof with the test compound; and determining whether the test compound specifically binds to the polypeptide, thereby determining that the test compound specifically binds to the polypeptide.

According to a further aspect, the invention provides a method for identifying a modulator of an NFkB activating activity. This method comprises contacting a polypeptide of the invention, or a fragment thereof, with a test compound; and measuring an activity of the NFkB activating polypeptide; wherein a change in NFkB activating activity measured in the presence of the test compound as compared to the NFkB activating activity in the absence of the test compound provides a determination that the test compound modulates an activity of the NFkB activating polypeptide. This method may further comprise providing two or more molecules that multimerize or specifically associate in the presence of an NFkB activating polypeptide, wherein the NFkB activating activity is measured by detecting an increase or decrease in the amount of multimerization or specific association of the molecules.

The methods can further comprise providing molecules that multimerize or specifically associate in the presence of an NFkB activating polypeptide and wherein the NFkB activating activity is measured by detecting an increase or decrease in the amount of multimerization or specific association of the molecules. In one aspect, a decrease in the amount of multimerization or specific association with the test compound as compared to the amount of multimerization or specific association without the test compound identifies the test compound as an inhibitor of NFkB activating polypeptide activity. An increase in the amount of multimerization or specific association with the test compound as compared to the amount of multimerization or specific association without the test compound can identify the test compound as an activator of NFkB activating polypeptide activity.

In one aspect, the test compound comprises a small molecule. The test compound can comprise an RNA. The RNA can comprise a small inhibitory RNA (siRNA) or an antisense RNA. The RNA can encode a protein in an NFkB-mediated activation pathway. The test compound can comprise a dominant negative form of a protein in an NFkB-mediated activation pathway.

The invention further provides a method for identifying a polypeptide able to upregulate the activity of an NFkB activity, said method comprising: contacting a polypeptide according to claim 20, or a fragment thereof, with a reporter gene with activity determined by the activation state of an NFkB; and detecting an increase in reporter gene activity; wherein an increase in the amount of reporter gene activity identifies a polypeptide able to upregulate the activity of NFkB. In one aspect, detecting the amount of reporter gene activity comprises detecting the amount of reporter gene transcript, the amount of polypeptide encoded by the reporter gene, or, where the reporter gene encodes an enzyme, detecting the amount of the enzyme's product or substrate.

The invention provides computer systems comprising a processor and a data storage device wherein said data storage device has stored thereon a polypeptide sequence or a nucleic acid sequence, wherein the polypeptide sequence comprises sequence of the invention, or a subsequence thereof, and the nucleic acid comprises a sequence of the invention or a subsequence thereof.

The invention provides computer readable mediums having stored thereon a polypeptide sequence or a nucleic acid sequence, wherein the polypeptide sequence comprises sequences of the invention, or a subsequence thereof, and the nucleic acid comprises sequences of the invention or a subsequence thereof.

The invention further provides a method for determining a functional fragment of an NFkB activating polypeptide. This method comprises deleting a plurality of amino acid residues from a polypeptide according to claim 20 to create a subsequence thereof; and testing the subsequence for an NFkB activating activity, thereby determining a functional fragment of an NFkB activating polypeptide.

According to another aspect of the present invention, there is provided a transgenic non-human animal comprising a heterologous nucleic acid, wherein the nucleic acid comprises a sequence having at least 90% sequence identity to SEQ ID NO:1 SEQ ID NO:3, or SEQ ID NO:4, wherein said animal exhibits a phenotype, relative to a wild-type phenotype comprising a characteristic selected from the group consisting of a dermatitis, a B cell defect, a T cell defect, and a combination of any two or more thereof. The transgenic non-human animal may be a mouse or a rat. Also provided is a cell or cell line derived from a transgenic non-human animal of the invention.

The invention provides transgenic non-human animals comprising a heterologous nucleic acid, wherein the nucleic acid comprises a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, the nucleic acid comprises a sequence that hybridizes under stringent conditions to a sequence as set forth in SEQ ID NO:1, SEQ ID NO:3 or SEQ ID NO:4, or a subsequence thereof, or the complement of any thereof.

The invention provides transgenic non-human animals comprising a nucleic acid of the invention flanked by a sequence to facilitate recombination, for example, a Cre/loxp sequence, wherein the nucleic acid comprises a sequence of the invention, e.g., a nucleic acid having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof.

The invention provides transgenic non-human animals comprising a vector comprising a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. In alternative aspects, the animal is a mouse or a rat. The expression of the gene sequence can inhibit expression of an endogenous NFkB activating polypeptide. Insertion of the gene sequence into the non-human animal's genome can inhibit expression of an endogenous NFkB activating polypeptide.

The invention provides transgenic plants or seeds comprising a nucleic acid of the invention, e.g., a nucleic acid sequence at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof.

According to a further aspect of the invention, there is provided a knockout non-human animal, wherein an endogenous gene sequence comprising a nucleic acid sequence having at least 90% sequence identity to SEQ ID NO:1 or SEQ ID NO:3 is disrupted so as to produce a phenotype comprising a characteristic selected from the group consisting of a dermatitis, a B cell defect, a T cell defect, and a combination of any two or more thereof. The animal may be a mouse or a rat. Also provided is a cell or cell line derived from a knockout non-human animal of the invention.

The invention provides knockout non-human animals wherein an endogenous gene sequence has been knocked out, wherein the knocked-out gene sequence comprises a nucleic acid of the invention, e.g., a nucleic acid sequence having at least 90% sequence identity to SEQ ID NO:1, SEQ ID NO:3 or SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:1, SEQ ID NO:3 or SEQ ID NO:4, or a subsequence thereof, or the complement of any thereof. The knockout transgenic non-human animal can further comprise a heterologous nucleic acid sequence having at least 90% sequence identity to SEQ ID NO:4 over a region of at least about 100 residues, wherein the sequence identities are determined by analysis with a sequence comparison algorithm or by visual inspection, or, a nucleic acid that hybridizes under stringent conditions to a nucleic acid sequence as set forth in SEQ ID NO:4, or a subsequence or complement thereof. The knock-out transgenic non-human animal can be a mouse or a rat.

The invention provides CARD11 knockout non-human animals made by a method, comprising knocking out the expression of an endogenous CARD11 gene sequence, wherein the gene sequence comprises a nucleic acid sequence having at least 90% sequence identity to SEQ ID NO:1, SEQ ID NO:3 or SEQ ID NO:4, wherein the sequence identities are determined by analysis with a sequence comparison algorithm or by visual inspection, or, a nucleic acid that hybridizes under stringent conditions to a nucleic acid sequence as set forth in SEQ ID NO:1, or a subsequence or complement thereof, and the endogenous gene sequence is knocked out by insertion of a nucleic acid of the invention, e.g., a nucleic acid sequence having at least 90% sequence identity to SEQ ID NO:1, SEQ ID NO:3 or SEQ ID NO:4, wherein the sequence identities are determined by analysis with a sequence comparison algorithm or by visual inspection, or, a nucleic acid that hybridizes under stringent conditions to a nucleic acid sequence as set forth in SEQ ID NO:1, or a subsequence or complement thereof. The CARD11 knockout non-human animal can be a mouse or a rat.

The invention provides inbred mouse or rat strains comprising a plurality of mice or rats of the invention, e.g., mice comprising a nucleic acid and/or polypeptide of the invention, e.g., transgenic mice.

The invention further provides a variety of methods using the transgenic non-human animals of the invention. The invention provides an in vitro method of screening for a modulator of an NFkB activating activity, said method comprising: contacting a cell or cell line derived from a transgenic animal with a test compound; and detecting an increase or a decrease in the amount of an NFkB reporter gene, an NFkB transcript, an NFkB protein, or an NFkB activity; thereby identifying the test compound as a modulator of an NFkB activating activity.

The invention provides an in vivo method of screening for a modulator of an NFkB activating activity, said method comprising: contacting a transgenic non-human animal of the invention with a test compound; and detecting an increase or a decrease in the amount of an NFkB reporter gene, an NFkB transcript, an NFkB protein, or an NFkB activity; thereby identifying the test compound as a modulator of an NFkB activating activity.

The invention provides an in vivo method for screening for a modulator of a dermatitis, a B cell defect, or a T cell defect, said method comprising: contacting a transgenic non-human animal of the invention with a test compound; and detecting an increase or a decrease in the amount or severity of the dermatitis, the B cell defect, or the T cell defect; wherein the increase or the decrease identifies the test compound as a modulator of the dermatitis, the B cell defect, or the T cell defect.

The invention provides an in vivo method to identify a genetic modulator of a dermatitis, a B cell defect, or a T cell defect, said method comprising: inserting a test gene into one or more cells of a transgenic non-human animal of the invention; and detecting an increase or a decrease in the amount or severity of the dermatitis, the B cell defect, or the T cell defect; wherein the increase or decrease identifies the test gene as a genetic modulator of the dermatitis, the B cell defect, or the T cell defect.

The invention provides an in vivo method to identify a genetic modulator of a dermatitis, a B cell defect, or a T cell defect, said method comprising: mating a first transgenic non-human animal of the invention with a second non-human animal of a sex opposite of the first transgenic non-human animal, wherein the second non-human animal is selected from the group consisting of an inbred non-human animal strain, a randomly mutagenized non-human animal, a transgenic non-human animal, and a knockout non-human animal; and selecting an offspring of the mating that exhibits an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first transgenic non-human animal of the invention with a second non-human animal of a sex opposite of the first transgenic non-human animal, wherein the second non-human animal is a randomly mutagenized non-human animal; (ii) mating two offspring of the mating of step (i); and (iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first transgenic non-human animal of the invention with a second non-human animal of a sex opposite of the first transgenic non-human animal, wherein the second non-human animal is a randomly mutagenized non-human animal; (ii) mating an offspring of the mating of step (i) with a transgenic non-human animal according to claim 35; and (iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first transgenic non-human animal of the invention with a second non-human animal of a sex opposite of the first transgenic non-human animal, wherein the second non-human animal is a randomly mutagenized non-human animal; (ii) mating an offspring of the mating of step (i) with a randomly mutagenized non-human animal; and (iii) identifying offspring of the mating of step (ii) that carry a mutated allele of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention further provides a variety of methods using the knockout non-human animals of the invention. The invention provides an in vivo method for screening for a modulator of a dermatitis, a B cell defect, or a T cell defect, said method comprising: contacting a knockout non-human animal of the invention with a test compound; and detecting an increase or a decrease in the amount or severity of the dermatitis, the B cell defect, or the T cell defect; wherein the increase or the decrease identifies the test compound as a modulator of the dermatitis, the B cell defect, or the T cell defect.

The invention provides an in vivo method to identify a genetic modulator of a dermatitis, a B cell defect, or a T cell defect, said method comprising: inserting a test gene into one or more cells of a knockout non-human animal of the invention; and detecting an increase or a decrease in the amount or severity of the dermatitis, the B cell defect, or the T cell defect; wherein the increase or decrease identifies the test gene as a genetic modulator of the dermatitis, the B cell defect, or the T cell defect.

According to a further aspect of the invention, there is provided an inbred mouse comprising a genome that is homozygous for a nucleic acid sequence encoding a polypeptide having at least 95% sequence identity to SEQ ID NO:2, wherein said polypeptide comprises a change in the amino acid sequence of SEQ ID NO:2 at amino acid residue number 298. The polypeptide comprises a sequence as set forth in SEQ ID NO:5. The inbred mouse has a phenotype comprising a characteristic selected from the group consisting of a dermatitis, a B cell defect, and a T cell defect. Also provided is a cell or cell line derived from an inbred mouse of the invention. The cell can comprises a plurality of B cells, a plurality of T cells or a combination thereof.

In one aspect, the mouse genome is homozygous for a CARD 11 polypeptide-coding sequence encoding a polypeptide comprising an amino acid sequence as set forth in SEQ ID NO:5. The mouse can have a phenotype comprising a dermatitis, a B cell defect, a T cell defect, an erythema or a combination thereof. The dermatitis can comprise an erythema of ear tissue, weepy eyes, dermatitis of skin on the neck, infiltration of eosinophils in or around inflamed areas of skin, elevated circulating levels of IgE, atopic dermatitis or a combination thereof.

In one aspect, the B cell defect comprises a guanylate kinase activity. In one aspect, the B cell defect comprises a defect in B cell signaling. The defect in B cell signaling can comprise a defect in B cell development, such as an arrest in splenic follicular B cell maturation. The defect in B cell signaling can comprise an inability to down-regulate surface IgM, a reduction in frequency of marginal zone and peritoneal B cells, a defective T dependent and T independent antibody response, a reduced mitogenic response after cross linking of B cell antigen receptors, a reduced mitogenic response to a combination of PMA and ionomycin or a combination thereof.

In one aspect, the T cell defect comprises a defective co-signal up-regulation of CD69 through CD28 signaling after T cell receptor (TCR) stimulation, a defective cell division response after CD28 signaling after T cell receptor (TCR) stimulation, a defective IL2 response after CD28 signaling after T cell receptor (TCR) stimulation, a defective IL2 receptor alpha chain response after CD28 signaling after T cell receptor (TCR) stimulation or a combination thereof.

The invention provides an inbred mouse strain comprising a plurality of inbred mice of the invention.

The invention provides an in vitro method of screening for a modulator of an NFkB activating activity, said method comprising: contacting a cell or cell line of the invention with a test compound; and detecting an increase or a decrease in the amount of an NFkB reporter gene, an NFkB transcript, an NFkB protein, or an NFkB activity; thereby identifying the test compound as a modulator of an NFkB activating activity. In one aspect, the test compound is a small molecule.

In one aspect, detecting the amount of reporter gene product produced comprises detecting the amount of reporter gene transcript, the amount of polypeptide encoded by the reporter gene, or, where the reporter gene encodes an enzyme, detecting the amount of the enzyme's product or substrate. The reporter gene can encodes a CD25, a TNF alpha, an interleukin 1, an interleukin 6, a CD25, a MIP1a, a MIP1b, a Bc1X1, an A1, an IRF4, a green fluorescent protein or a luciferase. In one aspect, an increase in the amount of the NFkB reporter gene identifies the test compound as an activator of the murine CARD11 NFkB activating activity. In one aspect, a decrease in the amount of the NFkB reporter gene identifies the test compound as an inhibitor of the murine CARD11 NFkB activating activity.

The invention provides an in vitro method for screening for a modulator of a murine CARD 11 NFkB activating activity comprising the following steps: (a) providing a cell of the invention or a cell line of the invention; (b) providing a test compound; (c) contacting the test compound of step (b) with the cell or cell line of step (a) and detecting an increase or a decrease in the amount of an NFkB transcript or an NFkB protein, wherein a decrease or an increase in the amount of the transcript or protein identifies the test compound as a modulator of CARD11 NFkB activating activity. In one aspect, the test compound is a small molecule.

The invention provides an in vitro method for screening for a modulator of a murine CARD11 NFkB activating activity comprising the following steps: (a) providing a cell of the invention or a cell line of the invention; (b) providing a test compound; (c) contacting the test compound of step (b) with the cell or cell line of step (a) and detecting an increase or a decrease in the amount of an NFkB activity, wherein a decrease or an increase in the amount of the NFkB activity identifies the test compound as a modulator of a CARD11 NFkB activating activity. In one aspect, the test compound is a small molecule.

In one aspect, the NFkB activating activity comprises recruitment of a signaling protein into a multi-molecular complex comprising a lipid raft. In one aspect, the NFkB activating activity comprises being a molecular scaffold for the assembly of a multiprotein complex. In one aspect, the NFkB activating activity comprises activation of IKK complex, degradation of p100 or phosphorylation of RelA (p65), c-Rel or RelB. In one aspect, the NFkB activating activity comprises stimulation of cellular apoptosis, cell proliferation, entry into mitosis or upregulation of NFkB-response polypeptides.

An in vivo method of screening for a modulator of an NFkB activating activity, said method comprising: contacting an inbred mouse of the invention with a test compound; and detecting an increase or a decrease in the amount of an NFkB reporter gene, an NFkB transcript, an NFkB protein, or an NFkB activity; thereby identifying the test compound as a modulator of an NFkB activating activity.

The method provides an in vivo method for screening for a modulator of a murine CARD11 NFkB activating activity comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of an NFkB reporter gene, wherein a decrease or an increase in the amount of the NFkB reporter gene identifies the test compound as an in vivo modulator of the murine CARD11 NFkB activating activity. In one aspect, the test compound is a small molecule.

In one aspect, detecting the amount of reporter gene product produced comprises detecting the amount of reporter gene transcript, the amount of polypeptide encoded by the reporter gene, or, where the reporter gene encodes an enzyme, detecting the amount of the enzyme's product or substrate. In one aspect, the reporter gene encodes a CD25, a TNF alpha, an interleukin 1, an interleukin 6, a CD25, a MIP1a, a MIP1b, a Bc1X1, an A1, an IRF4 or a luciferase.

In one aspect, an increase in the amount of the NFkB reporter gene identifies the test compound as an activator of the murine CARD11 NFkB activating activity. In one aspect, a decrease in the amount of the NFkB reporter gene identifies the test compound as an inhibitor of the murine CARD11 NFkB activating activity.

The invention further provides a variety of methods using an inbred mouse of the invention. The invention provides an in vivo method for screening for a modulator of a dermatitis, a B cell defect, a T cell defect, said method comprising: contacting an inbred mouse with a test compound; and detecting an increase or a decrease in the amount or severity of the dermatitis, the B cell defect, or the T cell defect; wherein the increase or the decrease identifies the test compound as a modulator of the dermatitis, the B cell defect, or the T cell defect.

The invention provides an in vivo method to identify a genetic modulator of a dermatitis, a B cell defect, or a T cell defect, said method comprising: inserting a test gene into one or more cells of an inbred mouse; and detecting an increase or a decrease in the amount or severity of the dermatitis, the B cell defect, or the T cell defect; wherein the increase or decrease identifies the test gene as a genetic modulator of the dermatitis, the B cell defect, or the T cell defect.

The invention provides an in vivo method to identify a genetic modulator of a dermatitis, a B cell defect, or a T cell defect, said method comprising: mating a first inbred mouse with a second mouse of a sex opposite of the first inbred mouse, wherein the second mouse is selected from the group consisting of an inbred mouse strain, a randomly mutagenized mouse, a transgenic mouse, and a knockout mouse; and selecting an offspring of the mating that exhibits an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first inbred mouse with a second mouse of a sex opposite of the first inbred mouse, wherein the second mouse is a randomly mutagenized non-human animal; (ii) mating two offspring of the mating of step (i); and (iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first inbred mouse with a second mouse of a sex opposite of the first inbred mouse, wherein the second mouse is a randomly mutagenized non-human animal; (ii) mating an offspring of the mating of step (i) with an inbred mouse; and (iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention provides an in vivo method to identify a genetic modulator of a dermatitis, a B cell defect, or a T cell defect, said method comprising: (i) mating a first inbred mouse with a second mouse of a sex opposite of the first inbred mouse, wherein the second mouse is a randomly mutagenized mouse; (ii) mating an offspring of the mating of step (i) with a randomly mutagenized mouse; and (iii) identifying offspring of the mating of step (ii) that carry a mutated allele of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

The invention provides an in vivo method for screening for a modulator of a murine CARD11 NFkB activating activity comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of an NFkB transcript or an NFkB protein, wherein a decrease or an increase in the amount of the transcript or protein identifies the test compound as a modulator of CARD11 NFkB activating activity. In one aspect, the test compound is a small molecule.

The invention provides an in vivo method for screening for a modulator of a murine CARD11 NFkB activating activity comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of an NFkB activity, wherein a decrease or an increase in the amount of the NFkB activity identifies the test compound as a modulator of a CARD11 NFkB activating activity. In one aspect, the test compound is a small molecule.

In one aspect, the NFkB activating activity comprises recruitment of a signaling protein into a multi-molecular complex comprising a lipid raft. In one aspect, the NFkB activating activity comprises being a molecular scaffold for the assembly of a multiprotein complex. In one aspect, the NFkB activating activity comprises activation of IKK complex, degradation of p100 or phosphorylation of RelA (p65), c-Rel or RelB. In one aspect, the NFkB activating activity comprises stimulation of cellular apoptosis, cell proliferation, entry into mitosis or upregulation of NFkB-response polypeptides.

The invention provides an in vivo method for screening for a modulator of a dermatitis comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of the dermatitis, wherein a decrease or an increase in the amount or severity of the dermatitis identifies the test compound as a modulator of the dermatitis. In one aspect, the dermatitis comprises an erythema, an erythema of ear tissue, weepy eyes, dermatitis of skin on the neck, infiltration of eosinophils in or around inflamed areas of skin, elevated circulating levels of IgE, atopic dermatitis or a combination thereof. In one aspect, the test compound is a small molecule.

The invention provides an in vivo method for screening for a modulator of a B cell defect comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of the B cell defect, wherein a decrease or an increase in the amount or severity of the B cell defect identifies the test compound as a modulator of the B cell defect. In one aspect, the B cell defect comprises a guanylate kinase activity. In one aspect, the B cell defect comprises a defect in B cell development. In one aspect, the defect in B cell development comprises an arrest in splenic follicular B cell maturation. In one aspect, the B cell defect comprises a defect in B cell signaling. The defect in B cell signaling can comprises an inability to down-regulate surface IgM, a reduction in frequency of marginal zone and peritoneal B cells, a defective T dependent and T independent antibody response, a reduced mitogenic response after cross linking of B cell antigen receptors, a reduced mitogenic response to a combination of PMA and ionomycin or a combination thereof. In one aspect, the test compound is a small molecule.

The invention provides an in vivo method for screening for a modulator of a T cell defect comprising the following steps: (a) providing an inbred mouse of the invention or an inbred mouse strain of the invention; (b) providing a test compound; (c) contacting the test compound of step (b) with the inbred mouse or inbred mouse strain of step (a) and detecting an increase or a decrease in the amount of the T cell defect, wherein a decrease or an increase in the amount or severity of the T cell defect identifies the test compound as a modulator of the T cell defect. The T cell defect can comprise a defective co-signal up-regulation of CD69 through CD28 signaling after T cell receptor (TCR) stimulation. a defective cell division response after CD28 signaling after T cell receptor (TCR) stimulation, a defective IL2 response after CD28 signaling after T cell receptor (TCR) stimulation, a defective IL2 receptor alpha chain response after CD28 signaling after T cell receptor (TCR) stimulation or a combination thereof.

The invention provides an in vivo method to screen for a genetic modulator of a dermatitis, a B cell defect or a T cell defect comprising the following steps: (a) providing an inbred mouse of the invention or an inbred mouse strain of the invention; (b) providing a test gene; (c) inserting the test gene of step (b) into one or more cells of a mouse of step (a); and (d) detecting an increase or a decrease in the amount of the dermatitis, B cell defect or T cell defect, wherein a decrease or an increase in the amount or severity of the dermatitis, B cell defect or T cell defect identifies the test gene as a genetic modulator of the dermatitis, B cell defect or T cell defect.

The invention provides an in vivo method to identify a genetic modulator of a dermatitis, a B cell defect or a T cell defect comprising the following steps: (a) providing a first mouse, wherein the mouse is an inbred mouse of the invention or an inbred mouse strain of the invention; (b) providing a second mouse of the sex opposite that of step (a), wherein the second mouse is an inbred mouse strain; (c) mating a first mouse of step (a) with a second mouse of step (b); and (d) selecting an offspring of the mating of step (c) that expresses an increased or a decreased amount of the dermatitis, B cell defect or T cell defect, thereby identifying a genetic modulator of a dermatitis, a B cell defect or a T cell defect. The method can further comprise identifying the gene that modulates the increased or a decreased amount of the dermatitis, B cell defect or T cell defect in the offspring mouse.

The invention provides an in vivo method to identify a genetic modulator of a dermatitis, a B cell defect or a T cell defect comprising the following steps: (a) providing a first mouse, wherein the mouse is an inbred mouse of the invention or an inbred mouse strain of the invention; (b) providing a second mouse of the sex opposite that of step (a), wherein the second mouse is a randomly mutagenized mouse; (c) mating a first mouse of step (a) with a second mouse of step (b); and (d) selecting an offspring of the mating of step (c) that expresses an increased or a decreased amount of the dermatitis, B cell defect or T cell defect, thereby identifying a genetic modulator of a dermatitis, a B cell defect or a T cell defect. The method can further comprise identifying the gene that modulates the increased or a decreased amount of the dermatitis, B cell defect or T cell defect in the offspring mouse.

The invention provides an in vivo method to identify a genetic modulator of a dermatitis, a B cell defect or a T cell defect comprising the following steps: (a) providing a first mouse, wherein the mouse is an inbred mouse of the invention or an inbred mouse strain of the invention; (b) providing a second mouse of the sex opposite that of step (a), wherein the second mouse is an transgenic or a knockout mouse; (c) mating a first mouse of step (a) with a second mouse of step (b); and (d) selecting an offspring of the mating of step (c) that expresses an increased or a decreased amount of the dermatitis, B cell defect or T cell defect, thereby identifying a genetic modulator of a dermatitis, a B cell defect or a T cell defect. The method can further comprise identifying the gene that modulates the increased or a decreased amount of the dermatitis, B cell defect or T cell defect in the offspring mouse.

The invention provides an in vivo method to identify a genetic modulator of a dermatitis, a B cell defect or a T cell defect comprising the following steps: (a) providing a first mouse, wherein the mouse is an inbred mouse of the invention or an inbred mouse strain of the invention; (b) providing a second mouse of a sex opposite that of step (a), wherein the second mouse is a randomly mutagenized mouse; (c) mating a first mouse of step (a) with a second mouse of step (b); (d) mating a first mouse of step (c) with a second mouse of step (c); (e) identifying mice from the litters of step (d) which carry two mutated CARD11 alleles; and (f) identifying mice from step (e) that have an altered severity of dermatitis, B cell defect or T cell defect as compared to the founder mouse strain of step (a). The mutated alleles can be detected by use of an allele-specific PCR reaction. In one aspect, the dermatitis comprises infiltration of eosinophils in or around inflamed areas of skin. The dermatitis can comprise elevated circulating levels of IgE. The dermatitis phenotype can comprise atopic dermatitis. The test compound can be a small molecule.

The invention provides in vivo methods for screening for a modulator of a B cell defect comprising the following steps: (a) providing a transgenic non-human animal of the invention; (b) providing a test compound; (c) contacting the test compound of step (b) with the non-human animal of step (a) and detecting an increase or a decrease in the amount of the B cell defect, wherein a decrease or an increase in the amount of the B cell defect identifies the test compound as a modulator of the B cell defect. In one aspect, the B cell defect comprises a guanylate kinase activity. The B cell defect can comprise a defect in B cell development, such as an arrest in splenic follicular B cell maturation, or, a defect in B cell signaling, or, an inability to down-regulate surface IgM, or, a reduction in frequency of marginal zone and peritoneal B cells, or, a defective T dependent and T independent antibody response, or, a reduced mitogenic response after cross linking of B cell antigen receptors, or, a reduced mitogenic response to a combination of PMA and ionomycin, or, all or a combination thereof. In one aspect, the test compound is a small molecule.

The invention provides in vivo methods for screening for a modulator of a T cell defect comprising the following steps: (a) providing a transgenic non-human animal of the invention; (b) providing a test compound; (c) contacting the test compound of step (b) with the non-human animal of step (a) and detecting an increase or a decrease in the amount of the T cell defect, wherein a decrease or an increase in the amount of the T cell defect identifies the test compound as a modulator of the T cell defect. In one aspect, the T cell defect comprises a defective co-signal up-regulation of CD69 through CD28 signaling after T cell receptor (TCR) stimulation, or, a defective cell division response after CD28 signaling after T cell receptor (TCR) stimulation, or, a defective IL2 response after CD28 signaling after T cell receptor (TCR) stimulation, or, a defective IL2 receptor alpha chain response after CD28 signaling after T cell receptor (TCR) stimulation, or, all or a combination thereof. The test compound can be a small molecule.

The invention provides in vivo methods to identify a genetic modulator of a dermatitis, a B cell defect, a T cell defect, or a lymphoma, comprising the following steps: (a) providing a transgenic non-human animal of the invention; (b) providing a test gene; (c) inserting the test gene of step (b) into one or more cells of a non-human animal of step (a); and (d) detecting an increase or a decrease in the amount of the dermatitis, B cell defect, T cell defect, or lymphoma, wherein a decrease or an increase in the amount or severity of the dermatitis, B cell defect, T cell defect, or lymphoma identifies the test gene as a genetic modulator of the dermatitis, B cell defect, T cell defect, or lymphoma.

The invention provides in vivo methods to identify a genetic modulator of a dermatitis, a B cell defect, a T cell defect, or a lymphoma, comprising the following steps: (a) providing a transgenic non-human animal of the invention; (b) providing a second non-human animal of a sex opposite that of step (a), wherein the second non-human animal is an inbred non-human animal strain; (c) mating a first non-human animal of step (a) with a second non-human animal of step (b); and (d) selecting an offspring non-human animal of the mating of step (c) that expresses an increased or a decreased amount of the dermatitis, B cell defect, T cell defect, or lymphoma, thereby identifying a genetic modulator of a dermatitis, a B cell defect, a T cell defect, or lymphoma. The method can further comprise identifying the gene that modulates the increased or a decreased amount of the dermatitis, B cell defect, T cell defect, or lymphoma in the offspring non-human animal.

The invention provides in vivo methods to identify a genetic modulator of a dermatitis, a B cell defect, a T cell defect, or a lymphoma, comprising the following steps: (a) providing a first transgenic non-human animal of the invention or an inbred mouse strain of the invention; (b) providing a second non-human animal of a sex opposite that of step (a), wherein the second non-human animal is a randomly mutagenized non-human animal; (c) mating a first non-human animal of step (a) with a second non-human animal of step (b); and (d) selecting an offspring of the mating of step (c) that expresses an increased or a decreased amount of the dermatitis, B cell defect, T cell defect, or lymphoma, thereby identifying a genetic modulator of a dermatitis, a B cell defect, a T cell defect, or lymphoma. The method can further comprise identifying the gene that modulates the increased or a decreased amount of the dermatitis, B cell defect, T cell defect, or lymphoma in the offspring. The method can further comprise identifying the mutation that modulates the increased or a decreased amount of the dermatitis, B cell defect, T cell defect, or lymphoma in the offspring.

The invention provides in vivo methods to identify a genetic modulator of a dermatitis, a B cell defect, a T cell defect, or a lymphoma, comprising the following steps: (a) providing a first transgenic non-human animal of the invention; (b) providing a second non-human animal of a sex opposite that of step (a), wherein the second non-human animal is an transgenic or a knockout non-human animal; (c) mating a first non-human animal of step (a) with a second non-human animal of step (b); and (d) selecting an offspring non-human animal of the mating of step (c) that expresses an increased or a decreased amount of the dermatitis, B cell defect, T cell defect, or lymphoma, thereby identifying a genetic modulator of a dermatitis, a B cell defect, a T cell defect, or a lymphoma. The method can further comprise identifying the gene that modulates the increased or a decreased amount of the dermatitis, B cell defect, T cell defect or lymphoma in the offspring non-human animal.

The invention provides in vivo methods to identify a genetic modulator of a dermatitis, a B cell defect, a T cell defect, or a lymphoma, comprising the following steps: (a) providing a first transgenic non-human animal of the invention, or an inbred mouse strain of the invention; (b) providing a second non-human animal of a sex opposite that of step (a), wherein the second non-human animal is a randomly mutagenized non-human animal; (c) mating a first non-human animal of step (a) with a second non-human animal of step (b); (d) mating a first non-human animal of step (c) with a second non-human animal of step (c); (e) identifying non-human animals from the litters of step (d) which carry two mutated CARD11 alleles; and (f) identifying non-human animals from step (e) that have an altered severity of dermatitis, B cell defect, T cell defect, or lymphoma, as compared to the founder non-human animal strain of step (a). In one aspect, the mutated alleles are detected by use of an allele-specific PCR reaction.

The invention provides in vivo methods to identify a genetic modulator of a dermatitis, a B cell defect, a T cell defect, or a lymphoma, comprising the following steps: (a) providing a first transgenic non-human animal of the invention, or an inbred mouse strain of the invention; (b) providing a second non-human animal of a sex opposite that of step (a), wherein the second non-human animal is a randomly mutagenized non-human animal; (c) mating a first non-human animal of step (a) with a second non-human animal of step (b); (d) mating a first non-human animal of step (c) with a second transgenic non-human animal of the invention, or an inbred mouse strain of the invention; (e) identifying non-human animals from the litters of step (d) which carry two mutated CARD11 alleles; and (f) identifying non-human animals from step (e) that have an altered severity of dermatitis, B cell defect, T cell defect, or lymphoma, as compared to the founder non-human animal strain of step (a). The mutated alleles can be detected by use of an allele-specific PCR reaction.

The invention provides in vivo methods to identify a genetic modulator of a dermatitis, a B cell defect, a T cell defect, or a lymphoma, comprising the following steps: (a) providing a first transgenic non-human animal of the invention, or an inbred mouse strain of the invention; (b) providing a second non-human animal of a sex opposite that of step (a), wherein the second non-human animal is a randomly mutagenized non-human animal; (c) mating a first non-human animal of step (a) with a second non-human animal of step (b); (d) mating a first non-human animal of step (c) with a second non-human animal of a sex opposite that of step (a), wherein the second non-human animal is the mutagenized non-human animal from step (b); (e) identifying non-human animals from the litters of step (d) which carry a mutated CARD11 allele; and (f) identifying non-human animals from step (e) that have an altered severity of dermatitis, B cell defect, T cell defect, or lymphoma, as compared to the founder non-human animal strain of step (a). In one aspect, the mutated alleles are detected by use of an allele-specific PCR reaction.

The invention provides methods for generating a toleragenic signal in a subject comprising the following steps: (a) providing an antisense polynucleotide of the invention; and

(b) administering to the subject an amount of antisense polynucleotide of step (a) sufficient to inhibit the expression of a CARD11 polypeptide, thereby generating a T cell defect comprising a defective co-signal up-regulation of CD69 through CD28 signaling after T cell receptor (TCR) stimulation, thereby generating a toleragenic signal in the subject.

The invention provides methods for generating a toleragenic signal in a subject comprising the following steps: (a) providing an antibody of the invention; and (b) administering to the subject an amount of the antibody of step (a) sufficient to inhibit the activity of a CARD11 polypeptide, thereby generating a T cell defect comprising a defective co-signal up-regulation of CD69 through CD28 signaling after T cell receptor (TCR) stimulation, thereby generating a toleragenic signal in the subject.

The invention provides methods for tolerizing a subject to an antigen comprising the following steps: (a) providing an antisense polynucleotide of the invention; (b) providing an antigen; (c) administering to the subject an amount of antisense polynucleotide of step (a) sufficient to inhibit the expression of a CARD11 polypeptide, thereby generating a T cell defect comprising a defective co-signal up-regulation of CD69 through CD28 signaling after T cell receptor (TCR) stimulation, thereby generating a toleragenic signal in the subject; and (d) administering the antigen to the subject, thereby tolerizing the subject to the antigen.

The invention provides methods for tolerizing a subject to an antigen comprising the following steps: (a) providing an antibody of the invention; (b) providing an antigen; (c) administering to the subject an amount of antibody of step (a) sufficient to inhibit the expression of a CARD11 polypeptide, thereby generating a T cell defect comprising a defective co-signal up-regulation of CD69 through CD28 signaling after T cell receptor (TCR) stimulation, thereby generating a toleragenic signal in the subject; and (d) administering the antigen to the subject, thereby tolerizing the subject to the antigen.

The invention provides methods for tolerizing a subject to an antigen comprising the following steps: (a) providing an small molecule inhibitor to a polypeptide of the invention, e.g., a polypeptide of the invention having an NFkB activating activity; (b) providing an antigen; (c) administering to the subject an amount of small molecule of step (a) sufficient to inhibit the expression of a CARD11 polypeptide, thereby generating a T cell defect comprising a defective co-signal up-regulation of CD69 through CD28 signaling after T cell receptor (TCR) stimulation, thereby generating a toleragenic signal in the subject; and (d) administering the antigen to the subject, thereby tolerizing the subject to the antigen.

The invention provides a method of ameliorating a defect in B cell signaling in a subject by gene therapy or replacement therapy of caspase recruitment domain 11 (CARD11) comprising the following steps: (a) providing a caspase recruitment domain 11 (CARD11) polypeptide or a nucleic acid encoding a caspase recruitment domain 11 (CARD11) polypeptide operably linked to a promoter, wherein the promoter is capable of expressing the nucleic acid in a cell of the subject; (b) administering the polypeptide or the nucleic acid in an amount sufficient to ameliorate the defect in B cell signaling in the subject. The caspase recruitment domain 11 (CARD11) polypeptide can be a mammalian caspase recruitment domain 11 (CARD11) polypeptide. In one aspect, the caspase recruitment domain 11 (CARD11) polypeptide is a human caspase recruitment domain 11 (CARD11) polypeptide (see, e.g., WO 01/40468). In one aspect of the method, the subject is a mammal, such as a human.

In one aspect, the nucleic acid further comprises a promoter. The nucleic acid can be administered as naked DNA. The nucleic acid can further comprise an expression vector. The nucleic acid can further comprise a recombinant virus, a plasmid, a phage, a phagemid, a cosmid, a fosmid, a bacteriophage or an artificial chromosome or a recombinant viral vector. The viral vector can comprise an adenovirus vector, a retroviral vector or an adeno-associated viral vector.

In one aspect, the retroviral vector comprises a murine leukemia virus (MuLV), an gibbon ape leukemia virus (GaLV), a Simian Immuno-deficiency virus (SIV), a human immuno deficiency virus (HIV), and combinations thereof. In one aspect, the viral vector comprises an baculoviridiae, a parvoviridiae, a picornoviridiae, a herpesveridiae, a poxyiridae, an adenoviridiae or a picornnaviridiae virus.

In one aspect, the nucleic acid further comprises a bacterial artificial chromosome (BAC), a plasmid, a bacteriophage P1-derived vector (PAC), a yeast artificial chromosome (YAC), a mammalian artificial chromosome (MAC).

In one aspect, the nucleic acid or the polypeptide is administered systemically, regionally, or locally. In one aspect, the nucleic acid or the polypeptide is targeted to an epithelial tissue. The epithelial tissue can be a lung or a tracheal epithelial tissue. The nucleic acid or the polypeptide can be delivered by a nasal route. The nucleic acid or the polypeptide can be administered nasally by inhalation, such as by aerosol or mist. In alternative aspects, the nucleic acids and/or the polypeptides are administered transmucosally, transdermally, transdermally as naked DNA, transdermally by bombardment, by micro-projectile bombardment, orally, as biodegradable microspheres or capsules, in liposomes or a combination thereof. In alternative aspects, the nucleic acids and/or the polypeptides are administered intraarterially, intrathecally (IT), intravenously (IV), parenterally or in the intra-pleural cavity or a combination thereof.

In one aspect, the B cell defect comprises a guanylate kinase activity. In one aspect, the B cell defect comprises a defect in B cell development. The defect in B cell development can comprise an arrest in splenic follicular B cell maturation, a defect in B cell signaling, an inability to down-regulate surface IgM, a reduction in frequency of marginal zone and peritoneal B cells, a defective T dependent and T independent antibody response, a reduced mitogenic response after cross linking of B cell antigen receptors, a reduced mitogenic response to a combination of PMA and ionomycin, an immunodeficiency, such as an immunodeficiency comprising an inability to generate a humoral response.

In one aspect, the polypeptide is administered intravenously (IV) at a dosage of about 0.01 mg/hr to about 1.0 mg/hr. In one aspect, the polypeptide is administered over a period of between about 1 to 6 hours.

The invention provides methods of ameliorating a dermatitis, a B cell defect, a T cell defect, or a lymphoma in a subject by gene therapy or replacement therapy of caspase recruitment domain 11 (CARD11) comprising the following steps: (a) providing a caspase recruitment domain 11 (CARD11) polypeptide or a nucleic acid encoding a caspase recruitment domain 11 (CARD11) polypeptide operably linked to a promoter, wherein the promoter is capable of expressing the nucleic acid in a cell of the subject; (b) administering the polypeptide or the nucleic acid in an amount sufficient to ameliorate the dermatitis, B cell defect, T cell defect or lymphoma in the subject.

The dermatitis can comprise an erythema of ear tissue, weepy eyes, dermatitis of skin on the neck, infiltration of eosinophils in or around inflamed areas of skin, elevated circulating levels of IgE, atopic dermatitis or a combination thereof.

The B cell defect can comprise a guanylate kinase activity. In one aspect, the B cell defect comprises a defect in B cell signaling. The defect in B cell signaling can comprise a defect in B cell development, such as an arrest in splenic follicular B cell maturation. The defect in B cell signaling can comprise an inability to down-regulate surface IgM, a reduction in frequency of marginal zone and peritoneal B cells, a defective T dependent and T independent antibody response, a reduced mitogenic response after cross linking of B cell antigen receptors, a reduced mitogenic response to a combination of PMA and ionomycin or a combination thereof.

The T cell defect can comprise a defective co-signal up-regulation of CD69 through CD28 signaling after T cell receptor (TCR) stimulation, a defective cell division response after CD28 signaling after T cell receptor (TCR) stimulation, a defective IL2 response after CD28 signaling after T cell receptor (TCR) stimulation, a defective IL2 receptor alpha chain response after CD28 signaling after T cell receptor (TCR) stimulation or a combination thereof.

The invention also provides methods for detecting the presence of or the prognosis of a B cell defect, a T cell defect, a lymphoma, or a dermatitis in a subject by detecting the level of expression of a caspase recruitment domain 11 (CARD11) gene comprising the following steps:

(a) providing a nucleic acid encoding a caspase recruitment domain 11 (CARD11) polypeptide or a nucleic acid that hybridizes under stringent conditions to a nucleic acid encoding a caspase recruitment domain 11 (CARD11) polypeptide or the complement thereof; (b) providing a tissue, cell or fluid sample from the subject, wherein the sample comprises a nucleic acid message; (c) contacting the nucleic acid of step (a) with the sample of step (b) under conditions wherein the nucleic acid of step (a) can specifically bind to or hybridize to a nucleic acid from the sample of step (b); and (d) detecting and measuring the amount of a specific binding or hybridizing of the nucleic acid of step (a) to a nucleic acid from the sample of step (b), wherein lower than normal levels of caspase recruitment domain 11 (CARD11) message in a subject detects or prognoses a B cell defect, a T cell defect, a lymphoma, or a dermatitis.

In one aspect, the caspase recruitment domain 11 (CARD11) polypeptide is a mammalian caspase recruitment domain 11 (CARD11) polypeptide, such as a human caspase recruitment domain 11 (CARD11) polypeptide. In one aspect, the subject is a mammal, such as a human.

In one aspect, the nucleic acid of step (a) is immobilized on an array, a slide, a capillary tube and the like.

The invention provides methods for detecting the presence or the or prognosis of a B cell defect, a T cell defect, a lymphoma, or a dermatitis in a subject by detecting expression of a caspase recruitment domain 11 (CARD11) gene comprising the following steps: (a) providing an amplification primer pair capable of amplifying a nucleic acid encoding a caspase recruitment domain 11 (CARD11) polypeptide or a subsequence thereof; (b) providing a tissue, cell or fluid sample from the subject, wherein the sample comprises a nucleic acid message; (c) contacting the amplification primer pair of step (a) with the sample of step (b) under conditions wherein the amplification primer pair of step (a) can specifically amplify a nucleic acid from the sample of step (b); and (d) detecting and measuring the amount of specific amplification product, wherein lower than normal levels of caspase recruitment domain 11 (CARD11) message in a subject detects or prognoses a B cell defect, a T cell defect, a lymphoma, or a dermatitis. In one aspect, the caspase recruitment domain 11 (CARD11) polypeptide is a human caspase recruitment domain 11 (CARD11) polypeptide. In one aspect, the subject is a mammal, such as a human.

The invention provides methods for detecting the presence of or prognosis of a B cell defect, a T cell defect, a lymphoma, or a dermatitis in a subject by sequencing a caspase recruitment domain 11 (CARD11) gene or message comprising the following steps: (a) providing a tissue, cell or fluid sample from the subject, wherein the sample comprises a nucleic acid gene or message; (b) sequencing a caspase recruitment domain 11 (CARD11) gene or message from the sample of step (a), wherein mutations, additions or deletions of nucleic acid residues in the caspase recruitment domain 11 (CARD11) gene or message as compared to wild type caspase recruitment domain 11 (CARD11) detects or prognoses a B cell defect, a T cell defect, a lymphoma, or a dermatitis in the subject. In one aspect, the caspase recruitment domain 11 (CARD11) polypeptide is a human caspase recruitment domain 11 (CARD11) polypeptide. In one aspect, the subject is a mammal, such as a human.

The invention provides methods to identify alleles of CARD11 that predispose to immunological diseases such as dermatitis, atopy, allergy, rheumatoid arthritis, type I diabetes, SLE, other autoimmune diseases by sequencing CARD11 in patient and control populations and looking for alleles enriched in patient groups.

The invention provides methods for detecting a B cell defect, a T cell defect, a lymphoma, or a dermatitis in a subject by detecting amplifications or deletions in a caspase recruitment domain 11 (CARD11) gene comprising the following steps: (a) providing a tissue, cell or fluid sample from the subject, wherein the sample comprises genomic DNA; (b) detecting and measuring a caspase recruitment domain 11 (CARD11) gene from the sample of step (a), wherein a deletion or an amplification of the caspase recruitment domain 11 (CARD11) gene detects or prognoses a B cell defect, a T cell defect, a lymphoma, or a dermatitis in the subject. In one aspect, the caspase recruitment domain 11 (CARD11) polypeptide is a human caspase recruitment domain 11 (CARD11) polypeptide. The subject can be a mammal, such as a human.

The method can further comprise providing an array comprising a immobilized nucleic acid, wherein the nucleic acid encodes a caspase recruitment domain 11 (CARD11) polypeptide or a nucleic acid that hybridizes under stringent conditions to a nucleic acid encoding a caspase recruitment domain 11 (CARD11) polypeptide, or the complement thereof, and the amplification or deletion is detected by contacting the sample of step (a) to the array-immobilized nucleic acid.

In one aspect, the amplification or deletion is detected by comparative genomic hybridization (CGH). The comparative genomic hybridization (CGH) can be an array-based CGH.

The invention provides methods for detecting a B cell defect, a T cell defect, a lymphoma, or a dermatitis in a subject by detecting amplifications or deletions in a caspase recruitment domain 11 (CARD11) gene comprising the following steps: (a) providing an amplification primer pair capable of amplifying a nucleic acid encoding a caspase recruitment domain 11 (CARD11) polypeptide or a subsequence thereof; (b) providing a tissue, cell or fluid sample from the subject, wherein the sample comprises a nucleic acid message; (c) contacting the amplification primer pair of step (a) with the sample of step (b) under conditions wherein the amplification primer pair of step (a) can specifically amplify a nucleic acid from the sample of step (b); and (d) detecting and measuring the amount of specific amplification product, wherein deletion or amplification of a caspase recruitment domain 11 (CARD11) gene in a subject detects a B cell defect, a T cell defect, a lymphoma or a dermatitis. In one aspect, the caspase recruitment domain 11 (CARD11) polypeptide is a human caspase recruitment domain 11 (CARD11) polypeptide. The subject can be a mammal, such as a human.

The invention provides methods for detecting a B cell defect, a T cell defect, a lymphoma, or a dermatitis in a subject by detecting caspase recruitment domain 11 (CARD11) polypeptide levels comprising the following steps: (a) providing a ligand capable of specifically binding to a caspase recruitment domain 11 (CARD11) polypeptide or a subsequence thereof; (b) providing a tissue, cell or fluid sample from the subject; (c) contacting the ligand of step (a) with the sample of step (b) under conditions wherein the ligand of step (a) can specifically bind to a caspase recruitment domain 11 (CARD11) polypeptide or fragment thereof from the sample of step (b); and (d) detecting and measuring the amount of caspase recruitment domain 11 (CARD11) polypeptide or fragment thereof, wherein an amount of caspase recruitment domain 11 (CARD11) polypeptide lower than normal levels in a subject detects or prognoses a B cell defect, a T cell defect, a lymphoma, or a dermatitis. The caspase recruitment domain 11 (CARD11) polypeptide can be a human caspase recruitment domain 11 (CARD11) polypeptide. The ligand can be an antibody that can specifically bind to a human caspase recruitment domain 11 (CARD11) polypeptide or a fragment thereof.

The invention provides methods further comprising providing an array comprising a immobilized ligand, wherein the ligand is capable of binding to a caspase recruitment domain 11 (CARD11) polypeptide, and the amount of caspase recruitment domain 11 (CARD11) polypeptide is detected by contacting the sample of step (a) to the array-immobilized ligand. The sample can comprise a body fluid, such as a CSF, urine or a blood sample, a biopsy, a sperm cell, a skin scraping and the like.

The invention further provides a method of treating an autoimmune disease or a lymphoma, said method comprising modulating a polypeptide having NFkB activating activity by administering a compound that binds to or modulates a polypeptide comprising (i) an amino acid sequence having at least 95% sequence identity to SEQ ID NO:2; or (ii) an amino acid sequence encoded by a nucleic acid comprising a sequence having at least 90% sequence identity to SEQ ID NO:1; wherein the polypeptide has an NFkB activating activity.

The details of one or more aspects of the invention are set forth in the accompanying drawings and the description below. Other features, objects, and advantages of the invention will be apparent from the description and drawings, and from the claims.

All publications, patents, patent applications, and GenBank sequences, cited herein are hereby expressly incorporated by reference for all purposes.

### DESCRIPTION OF DRAWINGS

The following drawings are illustrative of aspects of the invention and are not meant to limit the scope of the invention as encompassed by the claims.

FIG. 1 is an illustration of a two-dimensional display of representative data from two-color FACS experiments, where cells from the spleen and peritoneal cavity were stained with CD21 and CD23 and CD21 and IgM, respectively, as discussed in detail in Example 1, below.

FIG. 2 is a schematic of the protocol of a mixed bone marrow chimera experiment, as discussed in detail in Example 1, below.

FIG. 3 is an illustration of a two-dimensional display of representative data from two-color FACS experiments showing that the “unmodulated” mice B cell phenotype is cell-intrinsic, as discussed in detail in Example 1, below.

FIG. 4 is a graphic summary of representative data showing that “Unmodulated” mice B cells fail to respond to BCR crosslinking, as discussed in detail in Example 1, below.

FIG. 5 is a graphic summary of representative data showing that “Unmodulated” mice with dermatitis have very high levels of serum IgE, as discussed in detail in Example 1, below.

FIG. 6 is an illustration of representative results of a Western blot with anti-pTyr(4G10) antibody showing that early signaling events in “Unmodulated” mice B cells are defective, as shown by Western blot, as discussed in detail in Example 1, below.

FIG. 7 is a schematic of the mapping of the “pigsy” mutation, as discussed in detail in Example 1, below.

FIG. 8a illustrates the results of representative experiments where peripheral blood cells were stained for surface IgM and IgD; the percentage of cells stained indicated in quadrants. FIG. 8b and FIG. 8c illustrate B cell sub-populations in lymphoid organs of wild-type and mutant mice. FIG. 8d illustrates basal serum antibody levels in wild-type (●) and mutant (●) mice. FIG. 8e illustrates the effect of DNP-specific antibodies in sera prior to immunization (−) and post-immunization (+). FIG. 8f illustrates CGG-specific and Bordetella pertussis-specific IgG1 and IgG2a antibody responses after combined immunization. FIG. 8 is discussed in detail in Example 1, below.

FIG. 9a illustrates the results of representative experiments crosslinking B cell antigen receptors with antibodies to IgM. FIG. 9b illustrates the division of CFSE-labeled splenocytes from wildtype (grey) and unmodulated mice (black) stimulated for 72 hours. Shaded histograms show undivided cells in parallel cultures with IL-4 alone. FIG. 9c illustrates CD25 and CD86 levels on B cells after 16 hours stimulation. Shaded histograms represent unstimulated cells. Numbers indicate MFI. FIG. 9d illustrates protein tyrosine phosphorylation after stimulation with 10 μg/ml anti-IgM for times shown; lower panel: elevation of intracellular calcium, measured by Indo-1 fluorescence after stimulation with anti-IgM (arrow). FIG. 9e illustrates phosphorylation of ERK and JNK measured in purified B cells by Western blotting with anti-phosphoERK1/2 or anti-phosphoJNK antibodies. FIG. 9f illustrates BCR-induced degradation of IkB-alpha measured by Western blotting. FIG. 9g illustrates selective deficit in JNK and Bcl10 phosphorylation in purified B cells following stimulation with phorbol ester and ionomycin. Bcl10 is unfilled arrowhead; phospho Bcl10 is filled arrowhead. FIG. 9 is discussed in detail in Example 1, below.

FIG. 10a illustrates DNA synthesis after 48 hours in splenocytes from wildtype (filled symbols) and mutant (unfilled symbols) mice stimulated with the indicated concentrations of plate-bound anti-TCR in the presence (circles) or absence (triangles) of anti-CD28. FIG. 10b illustrates DNA synthesis in purified splenic T cells stimulated with plate-bound anti-TCR alone, see the left panel, ●:+/+, ∘:un/un. The right panel shows proliferation to 0.1 μg/ml anti-TCR without or with anti-CD28 co-stimulation. Mean and SD are shown for three mice of each genotype. FIG. 10c illustrates divisions of CFSE-labeled wild-type (unshaded) and mutant (shaded) CD4 or CD8 splenocytes after 72 hours co-culture stimulated with low concentration of anti-TCR plus anti-CD28 or with saturating concentration of anti-TCR. Dashed lines indicate CFSE in undivided cells. FIG. 10d illustrates induction of CD25 after 24 hours on CD4⁺ T cells stimulated as indicated (unshaded) or cultured without stimulation (shaded). Numbers represent MFI of CD25 on stimulated cells. FIG. 10e illustrates blastogenesis and intracellular IL-2 in co-cultured wild-type and mutant splenocytes after 22 hours stimulation with phorbol ester and ionomycin. Shaded histograms, unstimulated cells; open histograms, stimulated cells. The mean percentage ±SD of cells secreting IL-2 in wild-type cells (open bars) is compared with that of mutant cells (closed bars). FIG. 10 is discussed in detail in Example 1, below.

FIG. 11a illustrates the serum IgE concentration in WT, un/+ and mutant un/un mice of different ages. FIG. 11b illustrates representative histology (at 5× magnification) of wildtype and mutant (un/un) mouse skin with atopic dermatitis. FIG. 11 is discussed in detail in Example 1, below.

FIG. 12 shows illustrates aspects of the identification of the mutated gene in unmodulated mice. FIG. 12a illustrates high-resolution haplotype matrix representing a part of mouse chromosome 5. Numbers on the right column indicate the physical map position of SSLP markers based on public mouse genome assembly. FIG. 12b illustrates an electropherogram trace showing point-mutation and resulting codon substitution in unmodulated mice. Predicted amino-acid translation of nucleotide sequence adjacent point-mutation is shown in upper panel. A schematic diagram of mouse CARD11 structure and location of mutation is the lower panel. FIG. 12c illustrates partial amino-acid sequences of coiled-coil domain in mouse and human Carma proteins aligned. Residues that are conserved in 100% of analyzed proteins are boxed and shaded. The filled arrowhead indicates mutated leucine in unmodulated. Unfilled arrowheads indicate heptad repeats of hydrophobic amino acids. FIG. 12 is discussed in detail in Example 1, below.

FIG. 13 illustrates comparisons of primary and secondary lymphoid organ cellularity. Mice of 9-12 weeks of age and same sex were used for this analysis: wild-type (+/+, n=6, open bars), mutant heterozygotes (un/+, n=5, shaded bars) and mutant homozygotes (un/un, n=7, closed bars). FIG. 13 is discussed in detail in Example 1, below.

### DETAILED DESCRIPTION OF THE INVENTION

The invention provides novel caspase recruitment domain 11 (CARD11) polypeptides, nucleic acids encoding them and methods for making and using them. In one aspect, the polypeptides of the invention have NFkB activating activity. The invention also provides non-human animals, e.g., transgenics or inbred strains, comprising the CARD11 polypeptides and/or nucleic acids of the invention, and methods for making and using these animals. The invention provides in vitro and in vivo methods to identify genetic or chemical modulators of a CARD11 activity. The invention provides in vitro and in vivo methods to identify genetic or chemical modulators of a dermatitis, a B cell defect, a T cell defect, or a lymphoma. The invention also provides transgenic animals and inbred strains comprising non-human animals having their endogenous CARD11 activity disabled by using the nucleic acids of the invention, e.g., by use of antisense nucleic acids of the invention or by knockout of CARD11-encoding loci using nucleic acids of the invention.

The invention provides methods for identifying/screening for modulators (e.g., inhibitors, activators) of a CARD11 activity, e.g., an NFkB activating activity. The screening methods of the invention can be practiced in vitro or administered to a cell of the invention or an animal of the invention in vivo. Compounds (e.g., from combinatorial chemical libraries) are also screened using the compositions, cells, non-human animals and methods of the invention for their ability to ameliorate dermatitis, B cell defects and/or T cell defects, lymphoma, or for their ability to generate a toleragenic environment in an animal. The invention found that an animal that lacks sufficient CARD11 activity can have a dermatitis, a B cell defect and/or a T cell defect.

The B cell defect can involve a guanylate kinase activity. The B cell defect also can comprise a defect in B cell signaling, including, e.g., a defect in B cell development, an arrest in splenic follicular B cell maturation, an inability to down-regulate surface IgM, a reduction in frequency of marginal zone and peritoneal B cells, a defective T dependent and T independent antibody response, a reduced mitogenic response after cross linking of B cell antigen receptors or a reduced mitogenic response to a combination of PMA and ionomycin.

The T cell defect can comprise a defective co-signal up-regulation of CD69 through CD28 signaling after T cell receptor (TCR) stimulation, a defective cell division response after CD28 signaling after T cell receptor (TCR) stimulation, a defective IL2 response after CD28 signaling after T cell receptor (TCR) stimulation or a defective IL2 receptor alpha chain response after CD28 signaling after T cell receptor (TCR) stimulation.

Thus, the non-human animals of the invention and cells of the invention, including the inbred mouse strains of the invention, that have been engineered to express no or less than sufficient levels of active CARD11 polypeptide can be used as screening models for modulators of these CARD11 activities and for modulators of and agents for treatment and amelioration of these conditions. These methods can be used to screen for drugs to ameliorate/treat dermatitis and B cell and T cell defects.

The invention also provides pharmaceutical composition comprising a nucleic acid or polypeptide of the invention. Administration of a pharmaceutical composition of the invention to a subject is used to generate a toleragenic immunological environment in the subject. This can be used to tolerize the subject to an antigen.

The invention also provides inbred mouse strains homozygous for a non-wild type CARD11 allele (the “wild type” allele nucleic acid coding sequence, genomic sequence and polypeptide are set forth as SEQ ID NO:1, SEQ ID NO:3 and SEQ ID NO:2, respectively). This genotype results in mice having a phenotype comprising dermatitis, B cell defects and T cell defects. While the invention is not limited by any particular mechanism of action, in one aspect, the genotype of the inbred mice of the invention results in a changed NFkB activating activity. In alternative aspects, the changed NFkB activating activity involves CARD11 recruitment of a signaling protein into a multi-molecular complex comprising a lipid raft, CARD11 being a molecular scaffold for the assembly of a multiprotein complex, CARD11 involvement in an IKK complex, CARD11 involvement in the degradation of p100 or the phosphorylation of RelA (p65), c-Rel or RelB. While the invention is not limited by any particular mechanism of action, in alternative aspects the NFkB activating activity comprises stimulation of cellular apoptosis, cell proliferation, entry into mitosis or upregulation of NFkB-response polypeptides.

The CARD11 allele of the inbred strain has a single base pair residue change as compared to wild type. The changed, or mutant, sequence in the inbred mouse of the invention is at base residue number 907 in SEQ ID NO:1, where an ‘A’ is changed to a ‘T’ causing a change in nucleic acid subsequence “ACATCCTGGAACATGACC” to “ACATCCAGGAACATGACC” (see SEQ ID NO:4). This nucleic acid change results in a change of amino acid from leucine to glutamine at amino acid 298 of SEQ ID NO:5, and causing a change in amino acid subsequence ‘DILEH’ to ‘DIQEH’.

The invention also provides methods for in vitro and in vivo screening for modulators, e.g., inhibitors or activators, of a CARD11 mediated activity. The in vitro screening can include use of cell extracts. The in vivo screening can include single cell—(“whole cell”—) based screening and whole (non-human) animal screening. The whole animal screening methods include the transgenic and knockout animals of the invention and the inbred mice of the invention. Again, while the invention is not limited by any particular mechanism of action, in alternative aspects the invention provides in vitro and in vivo methods for screening for modulators of a CARD11 activity, e.g., a CARD11 NFkB activating activity, including a murine CARD11 NFkB activating activities.

An increase or a decrease in the amount of CARD11 activity can be detected in vitro and in vivo by measuring a decrease or an increase in the activity of a “reporter” gene, e.g., a coding sequence controlled by a CARD11-responsive transcriptional control element, e.g., an NFkB responsive promoter element, i.e., an “NFkB reporter gene.” In alternative aspects, the amount of reporter gene transcript, the amount of polypeptide encoded by the reporter gene, or, where the reporter gene encodes an enzyme, detecting the amount of the enzyme's product or substrate, or, where the reporter gene encodes a bioluminescent protein, the luminescence of the protein. The reporter gene can encode any directly or indirectly detectable transcript or polypeptide, e.g., a CD25, a TNF alpha, an interleukin 1, an interleukin 6, a CD25, a MIP1a, a MIP1b, a Bc1X1, an A1, an IRF4, a green fluorescent protein or a luciferase. Expression of a luciferase or a green fluorescent protein can be measured in vitro or in vivo by a variety of known devices, e.g., a luminometer (see, e.g., Lojek (2002) Luminescence 17:1-4; U.S. Pat. Nos. 6,377,342; 6,348,965), or by MRI (see, e.g., WO 02/47537).

Generating and Manipulating Nucleic Acids

The invention provides nucleic acids, including expression cassettes such as expression vectors, encoding the polypeptides of the invention. The invention also provides non-human animals comprising the nucleic acids of the invention. The invention also provides CARD11 knockout non-human animals (e.g., transgenic or inbred strains) made using the nucleic acids of the invention.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

The phrases “nucleic acid” or “nucleic acid sequence” as used herein refer to an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol.; 144:189-197; Strauss-Soukup (1997) Biochemistry 36:8692-8698; Samstag (1996) Antisense Nucleic Acid Drug Dev 6:153-156.

The invention provides isolated or recombinant nucleic acids comprising sequences having various sequence identities to SEQ ID NO:1, SEQ ID NO:3 or SEQ ID NO:4, or, sequences that hybridize under stringent conditions to these exemplary sequences of the nucleotide subsequences thereof, or the complement of any thereof. An exemplary CAR11 nucleotide sequence (SEQ ID NO:1) is as follows:

| | | |
|---|---|---|
| 1 | GAGGAGGGCCAGCTATGGATGACTACATGGAGACGCTGAAGGATGAAGAGGAGGCCCTAT | (SEQ ID NO:1) |
| 61 | GGGATAACGTGGAATGCAACCGGCACATGCTGAGCCGTTACATCAACCCCGCCAAGCTCA | |
| 121 | CCCCCTACCTGCGCCAGTGCAAGGTCATCGATGAGCAAGATGAAGACGAGGTGCTCAATG | |
| 181 | CGCCCATGCTGCCGTCCAAGATCAACCGTGCAGGCCGATTGTTGGACATTCTTCACACCA | |
| 241 | AGGGACAAAGGGGCTATGTGGTCTTCCTGGAGAGCCTGGAGTTTTACTACCCAGAACTTT | |
| 301 | ACAAACTGGTGACTGGAAAGGAACCCACCCGGAGATTCTCCACCATTGTGGTGGAGGAAG | |
| 361 | GCCATGAGGGCCTCACACACTTCCTGATGAACGAGGTCATCAAACTGCAGCAGCAAGTGA | |
| 421 | AAGCCAAGGACCTTCAGCGCTGTGAGCTGCTGGCCAAGTCCCGGCAACTGGAGGATGAGA | |
| 481 | AGAAGCAGCTGAGCCTGATACGGGTGGAGCTGCTGACCTTCCAGGAGCGATACTACAAGA | |
| 541 | TGAAGGAGGAGCGGGACAGCTACAATGACGAGCTCGTCAAGGTCAAGGACGACAACTACA | |
| 601 | ACTTAGCCATGCGCTACGCCCAGCTCAGTGAGGAGAAAAACATGGCGGTGATGAGGAGCC | |
| 661 | GCGACCTCCAACTCGAGATCGACCAGCTCAAACACCGACTGAACAAGATGGAGGAGGAAT | |
| 721 | GCAAGCTGGAGAGAAATCAGTCCCTCAAGCTCAAGAATGACATCGAGAACCGGCCCAGGA | |
| 781 | AGGAGCAGGTCCTGGAGCTGGAGCGGGAGAATGAGATGCTGAAGACGAAAATTCAGGAGC | |
| 841 | TGCAGTCCATCATCCAGGCTGGCAAGCGCAGCCTCCCTGACTCAGACAAGGCCATCTTGG | |
| 901 | ACATCCTGGAACATGACCGGAAGGAGGCGCTAGAGGACCGGCAGGAACTGGTCAACAAAA | |
| 961 | TTTACAACCTACAAGAGGAAGTCCGCCAGGCGGAGGAGCTGCGGGATAAGTACCTGGAGG | |
| 1021 | AGAAGGAAGACCTGGAACTCAAGTGTTCAACCCTGGGGAAGGACTGTGAAATGTACAAGC | |
| 1081 | ACCGCATGAACACAGTTATGCTGCAGCTGGAGGAGGTGGAGCGGGAGCGGGACCAGGCCT | |
| 1141 | TCCACTCCCGAGATGAGGCACAGACACAGTACTCACAGTGCTTAATCGAGAAGGACAAGT | |
| 1201 | ACCGGAAGCAGATCCGGGAGCTGGAGGAGAAGAACGATGAGATGCGTATTGAGATGGTGA | |
| 1261 | GGAGGGAGGCCTGTATTGTCAACCTGGAAAGCAAGCTCCGGCGCCTGTCCAAGGACAACG | |
| 1321 | GCAGCCTCGACCAGAGTCTGCCTAGACACCTTCCAGCCACCATCATCTCACAGAACCTTG | |
| 1381 | GAGACACCAGCCCCAGGACCAATGGCCAGGAAGCTGATGATTCTTCAACCTCAGAAGAGT | |
| 1441 | CTCCCGAAGACAGCAAGTACTTTCTGCCTTACCACCCACCCCGGCGCCGGATGAACCTAA | |
| 1501 | AGGGCATCCAGCTGCAGAGAGCCAAATCCCCCATCAGCATGAAGCAAGCATCTGAGTTTC | |
| 1561 | AAGTCAAGGGGCACGAAGAGGATTTCACAGACGGCAGCCCCAGTTCCTCCCGCTCGCTGC | |
| 1621 | CTGTCACCAGCTCTTTCTCCAAGATGCAACCCCATCGGAGCCGCAGCAGCATCATGTCAA | |
| 1681 | TCACGGCAGAGCCCCCGGGAAATGACTCCATAGTCAGACGCTGTAAGGAAGATGCGCCAC | |
| 1741 | ACCGGAGCACGGTGGAAGAAGACAACGATAGCTGTGGGTTTGATGCCTTAGACCTTGACG | |
| 1801 | ATGAAAATCACGAACGTTATTCCTTTGGACCTCCCTCCATCCACTCCTCCTCCTCTTCAC | |
| 1861 | ACCAGTCAGAGGGACTGGATGCCTACGACCTGGAGCAGGTCAACCTCATGTTACGAAAGT | |
| 1921 | TCTCTTTGGAAAGGCCCTTCCGGCCATCGGTCACATCTGGGGGTCACGTGCGGGGCACCG | |
| 1981 | GGCCCTTGGTCCAGCACACAACTCTGAATGGCGATGGGCTCATCACGCAGCTCACCCTTC | |
| 2041 | TGGGCGGCAATGCACGCGGGAGCTTCATTCACTCTGTCAAGCCAGGCTCACTGGCTGAGA | |
| 2101 | GGGCCGGACTGCGTGAGGGCCACCAACTCCTGCTGCTGGAAGGTTGCATCCGAGGCGAAA | |
| 2161 | GGCAGAGCGTTCCACTGGATGCGTGCACAAAAGAAGAGGCCCGTTGGACCATCCAGAGGT | |
| 2221 | GCAGTGGCCTCATCACTCTGCATTACAAGGTCAACCATGAAGGATACCGGAAGCTGCTGA | |
| 2281 | AGGAGATGGAGGATGGTCTGATCACATCAGGGGACTCGTTCTATATCCGCCTGAACCTGA | |
| 2341 | ACATCTCCAGCCAGCTGGATGCCTGCTCCATGTCCCTCAAGTGTGACGACGTGGTGCATG | |
| 2401 | TCCTAGACACCATGTACCAGGACAGGCACGAGTGGCTGTGTGCACGAGTCGACCCCTTCA | |
| 2461 | CTGACCAAGACCTGGACACGGGCACCATCCCCAGCTACAGCCGGGCTCAACAGCTTCTCC | |
| 2521 | TGGTGAAGCTCCAGCGGTTGGTTCACAGAGGCAACCGGGAAGAGGCAGACAGCGCTCACC | |
| 2581 | ACACCCTGCGCAGCCTCCGGAACACCCTGCAGCCCGAAGAGATGCTTTCGACGAGCGACC | |
| 2641 | CCCGAGTCAGCCCCCGCCTCTCCAGAGCGAGTTTCTTCTTTGGCCAGCTCCTGCAGTTTG | |
| 2701 | TCAGCCGGTCAGAAAACAAGTACAAAAGAATGAACAGCAATGAGCGCGTGAGAATCATCT | |
| 2761 | CTGGGAGTCCCCTGGGGAGCCTCTCCCGGTCCTCGCTGGATGCCACCAAACTCCTGACCG | |
| 2821 | AGAAGCATGAAGAACTGGATCCTGAGAATGAGCTCAGCCGGAACCTCACCCTGATCCCTT | |
| 2881 | ACAGCCTGGTGCGCGCTTTCCACTGTGAGCGCCGCAGGCCTGTGCTCTTCACGCCCACCA | |
| 2941 | TGCTGGCCAAGACATTGGTGCAGAAGCTGCTCAACTCAGGGGGTGCCATGGAGTTCACCA | |
| 3001 | TCTGCAAGTCAGATATTGTCACAAGAGATGAGTTCCTCCGAAAGCAGAAGACAGAGACCA | |
| 3061 | TCATCTACTCCCGGGAAAAGAACCCCAACACCTTTGAATGCATCGTCCCTGCCAACATTG | |
| 3121 | AGGCTGTGGCAGCCAAGAACAAACACTGCCTGCTGGAGGCTGGGATCGGCTGTGTGCGCG | |
| 3181 | ACCTGATCAAGTGCAAGGTGTACCCCATAGTGCTGCTCATCCGGGTGAGCGAGAAGAACA | |
| 3241 | TCAAACGGTTCAGGAAGCTGCTGCCGCGGCCAGAGACGGAAGAGGAATTCCTGCGAGTGT | |
| 3301 | GCAGGCTCAAAGAGAAGGAGCTGGAGGCGCTGCCCTGCCTCTACGCCACCGTGGAAGCTG | |
| 3361 | AGATGTGGAGCAGCGTGGAGGAGCTGCTGCGAGTCCTCAAAGACAAGATTGTAGAGGAGC | |
| 3421 | AGCGCAAGACCATCTGGGTGGACGAGGACCAGCTGTGAGCTTGTCTGGGTCTGACCTACA | |
| 3481 | CACAGACACACCGG | |

SEQ ID NO:1 encodes a protein having a sequence as set forth in SEQ ID NO:2, as described below.

An exemplary genomic sequence (SEQ ID NO:3) comprising SEQ ID NO:1 is as follows: (the intronic splice junctions are in normal type, exons are in bold, dots represent remaining intronic sequence)

| | |
|---|---|
| ...TGTTTCATAGAGGCTTTTTTATTAACCTTGTTGGGGTTTATGCCTCGCAGGAGGAGGGCCAGCTATGGATGACTA | (SEQ ID NO:3) |
| CATGGAGACGCTGAAGGATGAAGAGGAGGCCCTATGGGATAACGTGGAATGCAACCGGCACATGCTGAGCCGTTACATCAACC | |
| CCGCCAAGCTCACCCCCTACCTGCGCCAGTGCAAGGTCATCGATGAGCAAGATGAAGACGAGGTGCTCAATGCGCCCATGCTG | |
| CCGTCCAAGATCAACCGTGCAGGTAGTGGAGGGAAGGGAAGTGTGTGAACCACACATGCTCAGTCAAGGGCA......TGTCATCCA | |
| ATCAAACACACTCTCACCATCTGGATGGAACTTCTCCCCAGGCCGATTGTTGGACATTCTTCACACCAAGGGACAAAGGGGCT | |
| ATGTGGTCTTCCTGGAGAGCCTGGAGTTTTACTACCCAGACTTTACAAACTGGTGACTGGAAAGGAACCCACCCGGAGATTC | |
| TCCACCATTGTGGGTAAGTGGCTTTGCTACCAGGGGCAAGGGAACCCTAGTAGAAGGATGTGTG......CTCCGCCACTGCCCCAC | |
| ACCTCAGGTCTGACTGGGTACCCCTCTCCACAGTGGAGGAAGGCCATGAGGGCCTCACACACTTCCTGATGAACGAGGTCATC | |
| AAACTGCAGCAGCAAGTGAAAGCCAAGGACCTTCAGCGCTGTGAGCTGCTGGCCAAGTCCCGGCAACTGGAGGATGAGAAGAA | |
| GCAGCTGAGCCTGATACGGGTGGAGCTGCTGACCTTCCAGGAGCGATACTACAAGATGAAGGAGGAGCGGGACAGCTACAATG | |
| ACGAGCTCGTCAAGGTCAAGGACGACAACTACAACTTAGCCATGCGCTACGCCCAGCTCAGTGAGGAGAAAAACATGGCGGTG | |
| ATGAGGAGCCGCGACCTCCAACTCGAGGTGGGGATGCCTGGGCTCCGGCTGAACTGAGGGAAGGGAAAAGAAATGTCT......GCA | |
| TTCCTTGACAACCATCCTCACCCCTCTGCTGGCGTGCTTGTCTTCAGATCGACCAGCTCAAACACCGACTGAACAAGATGGAG | |
| GAGGAATGCAAGCTGGAGAGAAATCAGTCCCTCAAGCTCAAGAATGACATCGAGAACCGGCCCAGGAAGGAGCAGGTCCTGGA | |
| GCTGGAGCGGGAGAATGAGATGCTGAAGACGAAAATTCAGGAGCTGCAGTCCATCATCCAGGTGAGACGCACCACCCTTGTAT | |
| AGGGGAGGGCTAGGCGGGACAAGGTGGGT......TAGCTCCTTTGTCTTCCTCCTGTACTGATCTCTGCTCCTAACCCCACCAGGC | |
| TGGCAAGCGCAGCCTCCCTGACTCAGACAAGGCCATCTTGGACATCCTGGAACATGACCGGAAGGAGGCGCTAGAGGACCGGC | |
| AGGAACTGGTCAACAAAATTTACAACCTACAAGAGGAAGTCCGCCAGGCGGAGGAGCTGCGGGATAAGGTGGGAGTACTATGG | |
| GTCAGGAGAGCAGCAGCCAGCCAGTGCCTTTAACAG......CCCCTGGAAGACCCAGGATCTCATTGCTCTCTCGCATTGCTCTCT | |
| CGCAGTACCTGGAGGAGAAGGAAGACCTGGAACTCAAGTGTTCAACCCTGGGGAAGGACTGTGAAATGTACAAGCACCGCATG | |
| AACACAGTTATGCTGCAGcTGGAGGAGGTGGAGCGGGAGCGGGACCAGGTACGGTGCCACCCTGGACGTGGCAGACCGTGAGG | |
| ATGCCCAGCAGCGCT......CTACTTTACTTTAGTTTGCTTTGGGTGAACCCGTAGCTCTTCCTTGGCAGGCCTTCCACTCCCGAG | |
| ATGAGGCACAGACACAGTACTCACAGTGCTTAATCGAGAAGGACAAGTACCGGAAGCAGATCCGGGAGCTGGAGGAGAAGAAC | |
| GATGAGATGCGTATTGAGATGGTGAGGAGGGAGGCCTGTATTGTCAACCTGGAAAGCAAGCTCCGGCGCCTGTCCAAGGACAA | |
| CGGCAGCCTCGACCAGGTAGGCTTAGCTAGCCATGTCCCCATACCCATGGCCAGGTGTCCCCCAAT......unknownAGTCTGCC | |
| TAGACACCTTCCAGCCACCATCATCTCACAGAACCTTGGAGACACCAGCCCCAGGACCAATGGCCAGGAAGCTGATGATTCTT | |
| CAACCTCAGAAGAGTCTCCCGAAGACAGCAAGTACTTTCTGCCTTACCACCCACCCCGGCGCCGGATGAACCTAAAGGGCATC | |
| CAGCTGCAGAGunknown......TTGTGTCTTCTGTCTTGTGTCTTCCCCTCCTCTCTCTTTCAGCTGCAGAGAGCCAAATCCCCC | |
| ATCAGCATGAAGCAAGCATCTGAGTTTCAAGGTTAGTAGGCTGCCCAGATCCTTTTTGCCCTTGCTCATCTGTCACCCTCCC... | |
| ...CCCTTTTCTCTCTCCTCTGTTTTCTGGGCCCGCGGCTCTGATGAGGACAGTCAAGGGGCACGAAGAGGATTTCACAGACGGC | |
| AGCCCCAGTTCCTCCCGCTCGCTGCCTGTCACCAGCTCTTTCTCCAAGATGGTGAGCTGCCGTAATTTCCACTCCACTTACAC | |
| ACCTTCAGAGTCCCCGGG......CGAGGCATCGCAGATAAATATCCGGTAACACAGCTGTGCCGCATTCACAGCAACCCCATCGGA | |
| GCCGCAGCAGCATCATGTCAATCACGGCAGAGCCCCCGGGAAATGACTCCATAGTCAGACGCTGTAAGGAAGATGCGCCACAC | |
| CGGAGGTGAGTGAGTGGCAGCTGGAGGCCTTGGCTAGGTGACTGACCCTGTCTCCA......CGCCAGTGTCGTCTCACCGCCCCCC | |
| CTCCCCCCGGGCTTGTTTCTTACAGCACGGTGGAAGAAGACAACGATAGCTGTGGGTTTGATGCCTTAGACCTTGACGGTATG | |
| TATGTATCTGCCAGGCCCGAAGGAACCCCGGAGGGCAGGGTCTGC......CCCCCCCCCCCCAGTGCTCCAGGTCCTGACAGACCC | |
| TCATTCCTCTGCAGATGAAAATCACGAACGTTATTCCTTTGGACCTCCCTCCATCCACTCCTCCTCCTCTTCACACCAGTCAG | |
| AGGGACTGGATGCCTACGACCTGGAGCAGGTCAACCTCATGTTACGAAAGTTCTCTTTGGAAAGGTATGGAGGCAGGGCTGGG | |
| GAGATGACTCTGTGGGTGTAGCACTTGCCAC......TTCAGCTCTTCTCTTTTGAGTCCAACTGCCTATTTTATTTCCCACCTCAG | |
| GCCCTTCCGGCCATCGGTCACATCTGGGGGTCACGTGCGGGGCACCGGGCCCTTGGTCCAGCACACAACTCTGAATGGCGATG | |
| GGCTCATCACGCAGCTCACCCTTCTGGGCGGCAATGCACGCGGGAGCTTCATTCACTCTGTCAAGCCAGGCTCACTGGCTGAG | |
| AGGGCCGGACTGCGTGAGGGCCACCAACTCCTGCTGGTGAGACATAGAGGGAGAAGCTACGGGTTGTCCCCAGGCCCTCCACT | |
| TCT......TGCCCATCAGCAGAGCCAAAGAAAATGAACCTCTTGTTCTTGTCCTGTAGCTGGAAGGTTGCATCCGAGGCGAAAGGC | |
| AGAGCGTTCCACTGGATGCGTGCACAAAAGAAGAGGCCCGTTGGACCATCCAGAGGTGCAGTGGCCTCATCACTCTGCATTAC | |
| AAGGTCACCATGAAGGTAAACCTGGGCCGGACCTGGTCCACACAAGGGTAGGGTACAGGACGCATA....AGACGCGGTATCTC | |
| CAGGGTGCTTACATTTCTGGATTACGGTCTCCTCAGGATACCGGAAGCTGCTGAAGGAGATGGAGGATGGTCTGATCACATCA | |
| GGGGACTCGTTCTATATCCGCCTGAACCTGAACATCTCCAGCCAGCTGGATGCCTGCTCCATGTCCCTCAAGTGTGACGACGT | |
| GGTGCATGTCCTAGACACCATGTACCAGGACAGGCACGAGTGGCTGTGTGCACGAGTCGACCCCTTCACTGACCAAGACCTGG | |
| ACACGGGCACCATCCCCAGCTACAGCCGGTGAGTGGGGGATGGGCTCCCACACCACCCCAGGCCAGCAGCTCCCTCAGC......AC | |
| ACCTTGCCCAGTCATAGCTCTGAGGTTGCTCCCATGTCCCCTCCACAGGGCTCAACAGCTTCTCCTGGTGAAGCTCCAGCGGT | |
| TGGTTCACAGAGGCAACCGGGAAGAGGCAGACAGCGCTCACCACACCCTGCGCAGCCTCCGGGTAGGTACACAAAGACACACA | |
| CACACACAGCCCAGGCCCTGCTGCCACCA......TACTCGGGAGCCCTTTCCTCAGCCTGAGCTGATTGACAAACTCATTCCAGAA | |
| CACCCTGCAGCCCGAAGAGATGCTTTCGACGAGCGACCCCCGAGTCAGCCCCCGCCTCTCCAGAGCGAGTTTCTTCTTTGGCC | |
| AGCTCCTGCAGGTAAGGTTGGTGATCGGATGCCCACTGACTTTTCTGGCACAGTGTGCTGG.....CCTGCCCCCACCCTCTACCC | |
| CACCCTCCTACCACTGTCTCTTCTCCACAGTTTGTCAGCCGGTCAGAAAACAAGTACAAAAGAATGAACAGCAATGAGCGCGT | |
| GAGAATCATCTCTGGGAGTCCCCTGGGGAGCCTCTCCCGGTCCTCGCTGGATGCCACCAAACTCCTGACCGAGAAGCATGAAG | |
| GTGTGTGACGACCTCGAGGCCCCACCCCACAGCCCAGCAGGGGCATGTCT.....TGCCCTCGGGCAGGGCAGGCTCTAACAGCCC | |
| ACTGTTTCTGTTCTGTTAGAACTGGATCCTGAGAATGAGCTCAGCCGGAACCTCACCCTGATCCCTTACAGCCTGGTGCGCGC | |
| TTTCCACTGTGAGCGCCGCAGGCCTGTGCTCTTCACGCCCACCATGCTGGCCAAGACATTGGTGCAGAAGCTGCTCAACTCAG | |
| GGGGTGCCATGGAGTTCACCATCTGCAAGTCAGGTGAGCATGGCCAGGTGACAGACAGAGGGACACAGGCTTCGGCAGCCCAT | |
| .....CCACCACAAATAGCACTTGCCACCCTGGGCACTAACTCCCCTCTCTTCAGATATTGTCACAAGAGATGAGTTCCTCCGAAA | |
| GCAGAAGACAGAGACCATCATCTACTCCCGGGAAAAGAACCCCAACACCTTTGAATGCATCGTCCCTGCCAACATTGAGGCTG | |
| TGGCAGCCAAGGTGAGAGACCCTGGGTACTGGCCAGACTAAGGCCCTACCCAACCATCTCT.....AAGGACAGCTGGGTGGGCAC | |
| CACTGCTGATGGTGGCTGTTATACTTGCAGAACAAACACTGCCTGCTGGAGGCTGGGATCGGCTGTGTGCGCGACCTGATCAA | |
| GTGCAAGGTGTACCCCATAGTGCTGCTCATCCGGGTGAGCGAGAAGAACATCAAACGGTTCAGGTAAGGACACCCAGTCCTCA | |
| CACCTGCCACACACACACCACCCTTACTCA......CATGTCTTCCCCTGCCTGCTGTCTCTCCCTGCCCACCTCTCCACCCCCAGG | |
| AAGCTGCTGCCGCGGCCAGAGACGGAAGAGGAATTCCTGCGAGTGTGCAGGCTCAAAGAGAAGGAGCTGGAGGCGCTGCCCTG | |
| CCTCTACGCCACCGTGGAAGCTGAGATGTGGAGCAGCGTGGAGGAGCTGCTGCGAGTCCTCAAAGACAAGATTGTAGAGGAGC | |
| AGCGCAAGACCATCTGGGTGGACGAGGACCAGCTGTGAGCTTGTCTGGGTCTGACCTACACACAGACACACCGG... | |

The invention also provides the following exemplary CARD 11 variant (SEQ ID No:4) of SEQ ID NO:1, with a mutation at base 907:

| | | |
|---|---|---|
| 1 | GAGGAGGGCCAGCTATGGATGACTACATGGAGACGCTGAAGGATGAAGAGGAGGCCCTAT | (SEQ ID NO:4) |
| 61 | GGGATAACGTGGAATGCAACCGGCACATGCTGAGCCGTTACATCAACCCCGCCAAGCTCA | |
| 121 | CCCCCTACCTGCGCCAGTGCAAGGTCATCGATGAGCAAGATGAAGACGAGGTGCTCAATG | |
| 181 | CGCCCATGCTGCCGTCCAAGATCAACCGTGCAGGCCGATTGTTGGACATTCTTCACACCA | |
| 241 | AGGGACAAAGGGGCTATGTGGTCTTCCTGGAGAGCCTGGAGTTTTACTACCCAGAACTTT | |
| 301 | ACAAACTGGTGACTGGAAAGGAACCCACCCGGAGATTCTCCACCATTGTGGTGGAGGAAG | |
| 361 | GCCATGAGGGCCTCACACACTTCCTGATGAACGAGGTCATCAAACTGCAGCAGCAAGTGA | |
| 421 | AAGCCAAGGACCTTCAGCGCTGTGAGCTGCTGGCCAAGTCCCGGCAACTGGAGGATGAGA | |
| 481 | AGAAGCAGCTGAGCCTGATACGGGTGGAGCTGCTGACCTTCCAGGAGCGATACTACAAGA | |
| 541 | TGAAGGAGGAGCGGGACAGCTACAATGACGAGCTCGTCAAGGTCAAGGACGACAACTACA | |
| 601 | ACTTAGCCATGCGCTACGCCCAGCTCAGTGAGGAGAAAAACATGGCGGTGATGAGGAGCC | |
| 661 | GCGACCTCCAACTCGAGATCGACCAGCTCAAACACCGACTGAACAAGATGGAGGAGGAAT | |
| 721 | GCAAGCTGGAGAGAAATCAGTCCCTCAAGCTCAAGAATGACATCGAGAACCGGCCCAGGA | |
| 781 | AGGAGCAGGTCCTGGAGCTGGAGCGGGAGAATGAGATGCTGAAGACGAAAATTCAGGAGC | |
| 841 | TGCAGTCCATCATCCAGGCTGGCAAGCGCAGCCTCCCTGACTCAGACAAGGCCATCTTGG | |
| 901 | ACATCCAGGAACATGACCGGAAGGAGGCGCTAGAGGACCGGCAGGAACTGGTCAACAAAA | |
| 961 | TTTACAACCTACAAGAGGAAGTCCGCCAGGCGGAGGAGCTGCGGGATAAGTACCTGGAGG | |
| 1021 | AGAAGGAAGACCTGGAACTCAAGTGTTCAACCCTGGGGAAGGACTGTGAAATGTACAAGC | |
| 1081 | ACCGCATGAACACAGTTATGCTGCAGCTGGAGGAGGTGGAGCGGGAGCGGGACCAGGCCT | |
| 1141 | TCCACTCCCGAGATGAGGCACAGACACAGTACTCACAGTGCTTAATCGAGAAGGACAAGT | |
| 1201 | ACCGGAAGCAGATCCGGGAGCTGGAGGAGAAGAACGATGAGATGCGTATTGAGATGGTGA | |
| 1261 | GGAGGGAGGCCTGTATTGTCAACCTGGAAAGCAAGCTCCGGCGCCTGTCCAAGGACAACG | |
| 1321 | GCAGCCTCGACCAGAGTCTGCCTAGACACCTTCCAGCCACCATCATCTCACAGAACCTTG | |
| 1381 | GAGACACCAGCCCCAGGACCAATGGCCAGGAAGCTGATGATTCTTCAACCTCAGAAGAGT | |
| 1441 | CTCCCGAAGACAGCAAGTACTTTCTGCCTTACCACCCACCCCGGCGCCGGATGAACCTAA | |
| 1501 | AGGGCATCCAGCTGCAGAGAGCCAAATCCCCCATCAGCATGAAGCAAGCATCTGAGTTTC | |
| 1561 | AAGTCAAGGGGCACGAAGAGGATTTCACAGACGGCAGCCCCAGTTCCTCCCGCTCGCTGC | |
| 1621 | CTGTCACCAGCTCTTTCTCCAAGATGCAACCCCATCGGAGCCGCAGCAGCATCATGTCAA | |
| 1681 | TCACGGCAGAGCCCCCGGGAAATGACTCCATAGTCAGACGCTGTAAGGAAGATGCGCCAC | |
| 1741 | ACCGGAGCACGGTGGAAGAAGACAACGATAGCTGTGGGTTTGATGCCTTAGACCTTGACG | |
| 1801 | ATGAAAATCACGAACGTTATTCCTTTGGACCTCCCTCCATCCACTCCTCCTCCTCTTCAC | |
| 1861 | ACCAGTCAGAGGGACTGGATGCCTACGACCTGGAGCAGGTCAACCTCATGTTACGAAAGT | |
| 1921 | TCTCTTTGGAAAGGCCCTTCCGGCCATCGGTCACATCTGGGGGTCACGTGCGGGGCACCG | |
| 1981 | GGCCCTTGGTCCAGCACACAACTCTGAATGGCGATGGGCTCATCACGCAGCTCACCCTTC | |
| 2041 | TGGGCGGCAATGCACGCGGGAGCTTCATTCACTCTGTCAAGCCAGGCTCACTGGCTGAGA | |
| 2101 | GGGCCGGACTGCGTGAGGGCCACCAACTCCTGCTGCTGGAAGGTTGCATCCGAGGCGAAA | |
| 2161 | GGCAGAGCGTTCCACTGGATGCGTGCACAAAAGAAGAGGCCCGTTGGACCATCCAGAGGT | |
| 2221 | GCAGTGGCCTCATCACTCTGCATTACAAGGTCAACCATGAAGGATACCGGAAGCTGCTGA | |
| 2281 | AGGAGATGGAGGATGGTCTGATCACATCAGGGGACTCGTTCTATATCCGCCTGAACCTGA | |
| 2341 | ACATCTCCAGCCAGCTGGATGCCTGCTCCATGTCCCTCAAGTGTGACGACGTGGTGCATG | |
| 2401 | TCCTAGACACCATGTACCAGGACAGGCACGAGTGGCTGTGTGCACGAGTCGACCCCTTCA | |
| 2461 | CTGACCAAGACCTGGACACGGGCACCATCCCCAGCTACAGCCGGGCTCAACAGCTTCTCC | |
| 2521 | TGGTGAAGCTCCAGCGGTTGGTTCACAGAGGCAACCGGGAAGAGGCAGACAGCGCTCACC | |
| 2581 | ACACCCTGCGCAGCCTCCGGAACACCCTGCAGCCCGAAGAGATGCTTTCGACGAGCGACC | |
| 2641 | CCCGAGTCAGCCCCCGCCTCTCCAGAGCGAGTTTCTTCTTTGGCCAGCTCCTGCAGTTTG | |
| 2701 | TCAGCCGGTCAGAAAACAAGTACAAAAGAATGAACAGCAATGAGCGCGTGAGAATCATCT | |
| 2761 | CTGGGAGTCCCCTGGGGAGCCTCTCCCGGTCCTCGCTGGATGCCACCAAACTCCTGACCG | |
| 2821 | AGAAGCATGAAGAACTGGATCCTGAGAATGAGCTCAGCCGGAACCTCACCCTGATCCCTT | |
| 2881 | ACAGCCTGGTGCGCGCTTTCCACTGTGAGCGCCGCAGGCCTGTGCTCTTCACGCCCACCA | |
| 2941 | TGCTGGCCAAGACATTGGTGCAGAAGCTGCTCAACTCAGGGGGTGCCATGGAGTTCACCA | |
| 3001 | TCTGCAAGTCAGATATTGTCACAAGAGATGAGTTCCTCCGAAAGCAGAAGACAGAGACCA | |
| 3061 | TCATCTACTCCCGGGAAAAGAACCCCAACACCTTTGAATGCATCGTCCCTGCCAACATTG | |
| 3121 | AGGCTGTGGCAGCCAAGAACAAACACTGCCTGCTGGAGGCTGGGATCGGCTGTGTGCGCG | |
| 3181 | ACCTGATCAAGTGCAAGGTGTACCCCATAGTGCTGCTCATCCGGGTGAGCGAGAAGAACA | |
| 3241 | TCAAACGGTTCAGGAAGCTGCTGCCGCGGCCAGAGACGGAAGAGGAATTCCTGCGAGTGT | |
| 3301 | GCAGGCTCAAAGAGAAGGAGCTGGAGGCGCTGCCCTGCCTCTACGCCACCGTGGAAGCTG | |
| 3361 | AGATGTGGAGCAGCGTGGAGGAGCTGCTGCGAGTCCTCAAAGACAAGATTGTAGAGGAGC | |
| 3421 | AGCGCAAGACCATCTGGGTGGACGAGGACCAGCTGTGAGCTTGTCTGGGTCTGACCTACA | |
| 3481 | CACAGACACACCGG | |

SEQ ID NO:4 encodes a protein having a sequence as set forth in SEQ ID NO:5, as described below.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which may be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll (1993) DNA Cell. Biol., 12:441-53.

Transcriptional Control Elements

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Expression Vectors and Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif. 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (AAV)-based vectors can be used to transduce cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada (1996) Gene Ther. 3:957-964.

The term “expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Host Cells and Transformed Cells

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the exemplary SEQ ID NO:1, SEQ ID NO:3 or SEQ ID NO:4, or subsequences thereof.

Amplification methods include, e.g., polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Hybridization of Nucleic Acids

The invention provides isolated or recombinant nucleic acids that hybridize under stringent conditions to an exemplary sequence of the invention, e.g., a sequence as set forth in SEQ ID NO:1, SEQ ID NO:3, SEQ ID NO:4, or the complement of any thereof, or a nucleic acid that encodes a polypeptide of the invention. In alternative aspects, the stringent conditions are highly stringent conditions, medium stringent conditions or low stringent conditions, as known in the art and as described herein. These methods may be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Hybridization” refers to the process by which a nucleic acid strand joins with a complementary strand through base pairing. Hybridization reactions can be sensitive and selective so that a particular sequence of interest can be identified even in samples sin which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C. Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 ng/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na⁺ concentration of approximately 1 M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

Oligonucleotides Probes and Methods for using them

The invention also provides nucleic acid probes for identifying nucleic acids encoding a polypeptide with an NFkB activating activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

Determining the Degree of Sequence Identity

The invention provides nucleic acids having at least 90%, 91%, 92%, 93%, 94%, 95%, 96%, 97%, 98%, 99% or more sequence identity to SEQ ID NO:1, SEQ ID NO:3, or SEQ ID NO:4. The invention provides polypeptides having at least 90%, 91%, 92%, 93%, 94%, 95%, 96%, 97%, 98%, 99% or more sequence identity to SEQ ID NO:2 or SEQ ID NO:5. The sequence identities can be determined by analysis with a sequence comparison algorithm or by a visual inspection.

Protein and/or nucleic acid sequence identities (homologies) may be evaluated using any of the variety of sequence comparison algorithms and programs known in the art. The extent of sequence identity (homology) may be determined using any computer program and associated parameters, including those described herein, such as BLAST 2.2.2. or FASTA version 3.0t78, with the default parameters. For example, the sequence comparison algorithm is a BLAST version algorithm. In one aspect, for nucleic acid sequence identity analysis, the BLAST nucleotide parameters comprise word size=11, expect=10, filter low complexity with DUST, cost to open gap=5, cost to extend gap=2, penalty for mismatch=−3, reward for match=1, Dropoff (X) for BLAST extensions in bits=20, final X dropoff value for gapped alignment=50, and all other options are set to default. In one aspect, for polypeptide sequence identity analysis the sequence comparison algorithm is a BLAST version algorithm, e.g., where the BLAST nucleotide parameters comprise word size=3, expect=10, filter low complexity with SEG, cost to open gap=11, cost to extend gap=1, similarity matrix Blosum62, Dropoff (X) for blast extensions in bits=7, X dropoff value for gapped alignment (in bits)=15, final X dropoff value for gapped alignment=25.

Exemplary algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993). Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin &Altschul (1993) Proc. Natl. Acad. Sci. USA90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “—F F” setting is used, which disables filtering. Use of default filtering often results in Karlin-Altschul violations due to short length of sequence.

The default values used in this exemplary aspect of the invention include:

“Filter for low complexity: ON

Word Size: 3

Matrix: Blosum62

Gap Costs: Existence: 1

Extension: 1”

Other default settings are: filter for low complexity OFF, word size of 3 for protein, BLOSUM62 matrix, gap existence penalty of —11 and a gap extension penalty of −1.

The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence SEQ ID NO:1, SEQ ID NO:2, SEQ ID NO:3, SEQ ID NO:4, SEQ ID NO:5) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, e.g., SEQ ID NO:1, SEQ ID NO:2, SEQ ID NO:3, SEQ ID NO:4, SEQ ID NO:5, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to SEQ ID NO:1, SEQ ID NO:2, SEQ ID NO:3, SEQ ID NO:4, SEQ ID NO:5, that sequence is within the scope of the invention.

The phrase “substantially identical” in the context of two nucleic acids or polypeptides, can refer to two or more sequences that have, e.g., at least about at least 90%, 91%, 92%, 93%, 94%, 95%, 96%, 97%, 98%, 99% or more sequence identity or more nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention, e.g., SEQ ID NO:1, SEQ ID NO:2, SEQ ID NO:3, SEQ ID NO:4, SEQ ID NO:5, over a region of at least about 10, 20, 30, 40, 50, 100, 150, 200, 250, 300, 350, 400, 450, 500 or more residues, or a region ranging from between about 50 residues to the full length of the nucleic acid or polypeptide. Nucleic acid sequences of the invention can be substantially identical over the entire length of a polypeptide coding region.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

Inhibiting Expression of Polypeptides and Transcripts

The invention further provides for nucleic acids complementary to (e.g., antisense sequences to) the nucleic acid sequences of the invention. Antisense sequences are capable of inhibiting the transport, splicing or transcription of protein-encoding genes, e.g., the CARD11 polypeptide encoding nucleic acids of the invention. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One may screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3:737 (2002). In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

Antisense Oligonucleotides

The invention provides antisense oligonucleotides capable of binding polypeptide message which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold (1995) J. of Biol. Chem. 270:13581-13584).

siRNA

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism (Elbashir et al., Genes Dev., 15(2): 188-200 (2001)). First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

Inhibitory Ribozymes

The invention provides ribozymes capable of binding message which can inhibit polypeptide activity by targeting mRNA, e.g., inhibition of polypeptides with CARD11 activity, e.g., NFkB activating activity. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are described by Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

Transgenic and “Knockout” Non-Human Animals

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode mammalian kinases. Native expression in an animal may be reduced by providing an amount of antisense RNA or DNA effective to reduce expression of the receptor.

These animals can be used, e.g., as in vivo models to study NFkB activating activity, or, as models to screen for agents that change the NFkB activating activity in vivo.

In one aspect, the inserted transgenic sequence is a sequence of the invention designed such that it does not express a functional CARD11 (NFkB activating) polypeptide. The defect can be designed to be on the transcriptional, translational and/or the protein level.

The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP). One exemplary method to produce genetically altered non-human animals is to genetically modify embryonic stem cells. The modified cells are injected into the blastocoel of a blastocyst. This is then grown in the uterus of a pseudopregnant female. In order to readily detect chimeric progeny, the blastocysts can be obtained from a different parental line than the embryonic stem cells. For example, the blastocysts and embryonic stem cells may be derived from parental lines with different hair color or other readily observable phenotype. The resulting chimeric animals can be bred in order to obtain non-chimeric animals which have received the modified genes through germ-line transmission. Techniques for the introduction of embryonic stem cells into blastocysts and the resulting generation of transgenic animals are well known.

Because cells contain more than one copy of a gene, the cell lines obtained from a first round of targeting are likely to be heterozygous for the targeted allele. Homozygosity, in which both alleles are modified, can be achieved in a number of ways. In one approach, a number of cells in which one copy has been modified are grown. They are then subjected to another round of targeting using a different selectable marker. Alternatively, homozygotes can be obtained by breeding animals heterozygous for the modified allele, according to traditional Mendelian genetics. In some situations, it may be desirable to have two different modified alleles. This can be achieved by successive rounds of gene targeting or by breeding heterozygotes, each of which carries one of the desired modified alleles. See, e.g., U.S. Pat. No. 5,789,215.

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 244:1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281-1288, 1989; and Simms et al., Bio/Technology 6:179-183, 1988).

CARD11 Functional Knockouts

The invention provides non-human animals that do not express their endogenous CARD11 polypeptides, or, express their endogenous CARD11 polypeptide at lower than wild type levels (thus, while not completely “knocked out” their CARD11 activity is functionally “knocked out”). The invention also provides “knockout animals” and methods for making and using them. For example, in one aspect, the transgenic or modified animals of the invention comprise a “knockout animal,” e.g., a “knockout mouse,” engineered not to express an endogenous gene, e.g., an endogenous CARD11 gene, which is replaced with a gene expressing a polypeptide of the invention, or, a fusion protein comprising a polypeptide of the invention. Thus, in one aspect, the inserted transgenic sequence is a sequence of the invention designed such that it does not express a functional CARD11 (NFkB activating) polypeptide. The defect can be designed to be on the transcriptional, translational and/or the protein level. Because the endogenous CARD11 gene has been “knocked out,” only the inserted polypeptide of the invention is expressed.

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration may be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 5,616,491 describes knock-out mice having suppression of CD28 and CD45. Procedures for preparation and manipulation of cells and embryos are similar to those described above with respect to transgenic animals, and are well known to those of ordinary skill in the art.

A knock out construct refers to a uniquely configured fragment of nucleic acid which is introduced into a stem cell line and allowed to recombine with the genome at the chromosomal locus of the gene of interest to be mutated. Thus, a given knock out construct is specific for a given gene to be targeted for disruption. Nonetheless, many common elements exist among these constructs and these elements are well known in the art. A typical knock out construct contains nucleic acid fragments of about 0.5 kb to about 10.0 kb from both the 5′ and the 3′ ends of the genomic locus which encodes the gene to be mutated. These two fragments are typically separated by an intervening fragment of nucleic acid which encodes a positive selectable marker, such as the neomycin resistance gene. The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug.

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of the polypeptide product of the wild-type gene from which it was derived.

Each knockout construct to be inserted into the cell must first be in the linear form. Therefore, if the knockout construct has been inserted into a vector, linearization is accomplished by digesting the DNA with a suitable restriction endonuclease selected to cut only within the vector sequence and not within the knockout construct sequence. For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipette and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan. After the ES cell has been introduced into the embryo, the embryo may be implanted into the uterus of a pseudopregnant foster mother for gestation as described above.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

The functional CARD11 “knockout” non-human animals of the invention are of several types. Some non-human animals of the invention that are functional CARD11 “knockouts” express sufficient levels of a CARD11 inhibitory nucleic acid, e.g., antisense sequences or ribozymes of the invention, to decrease the levels or knockout the expression of functional polypeptide. Some non-human animals of the invention that are functional CARD11 “knockouts” express sufficient levels of a CARD11 dominant negative polypeptide such that the effective amount of free endogenous active CARD11 is decreased. Some non-human animals of the invention that are functional CARD11 “knockouts” express sufficient levels of an antibody of the invention, e.g., a CARD11 antibody, such that the effective amount of free endogenous active CARD11 is decreased. Some non-human animals of the invention that are functional CARD11 “knockouts” are “conventional” knockouts in that their endogenous CARD11 gene has been disrupted or mutated.

Functional CARD11 “knockout” non-human animals of the invention also include the inbred mouse strain of the invention and the cells and cell lines derived from these mice.

The invention provides a novel use for these non-human animals by discovering that animals that do not express sufficient levels of a CARD11 polypeptide have dermatitis, a B cell defect and/or a T cell defect. Thus, by using the transgenic non-human animals or inbred strains, e.g., mouse strains, of the invention the invention provides in vivo methods to identify modulators, e.g., chemical or genetic modulators, of a dermatitis, a B cell defect or a T cell defect.

The dermatitis can comprise erythema, e.g., erythema of ear tissue. The dermatitis can comprise weepy eyes or dermatitis of the skin, e.g., dermatitis of the skin on the neck. In one aspect, the dermatitis comprises infiltration of eosinophils in or around inflamed areas of skin. The dermatitis can comprise elevated circulating levels of IgE. The dermatitis phenotype can comprise atopic dermatitis. The test compound can be a small molecule. Thus, the invention provides in vivo methods to identify in vivo modulators or drugs, e.g., chemical or genetic modulators, to treat or ameliorate elevated circulating levels of IgE, infiltration of eosinophils in inflamed areas of skin and/or dermatitis, e.g., an atopic dermatitis.

The B cell defect can comprise a defect in B cell development, such as an arrest in splenic follicular B cell maturation, or, a defect in B cell signaling, or, an inability to down-regulate surface IgM, or, a reduction in frequency of marginal zone and peritoneal B cells, or, a defective T dependent and T independent antibody response, or, a reduced mitogenic response after cross linking of B cell antigen receptors, or, a reduced mitogenic response to a combination of PMA and ionomycin, or, all or a combination thereof. Thus, the invention provides in vivo methods to identify in vivo modulators or drugs, e.g., chemical or genetic modulators, to treat or ameliorate an inability to down-regulate surface IgM, a reduction in frequency of marginal zone and peritoneal B cells, defective T dependent and T independent antibody response, a reduced mitogenic response after cross linking of B cell antigen receptors and/or a reduced mitogenic response.

The T cell defect can comprise a defective co-signal up-regulation of CD69 through CD28 signaling after T cell receptor (TCR) stimulation, a defective cell division response after CD28 signaling after T cell receptor (TCR) stimulation, a defective IL2 response after CD28 signaling after T cell receptor (TCR) stimulation, a defective IL2 receptor alpha chain response after CD28 signaling after T cell receptor (TCR) stimulation, or, all or a combination thereof. The invention provides in vitro or in vivo methods to identify in vivo modulators or drugs, e.g., chemical or genetic modulators, to treat or ameliorate defective co-signal up-regulation of CD69, e.g., through CD28 signaling after T cell receptor (TCR) stimulation, defective cell division response after CD28 signaling after T cell receptor (TCR) stimulation, defective IL2 response after CD28 signaling after TCR stimulation and/or defective IL2 receptor alpha chain response after CD28 signaling after TCR stimulation. Thus, the invention provides in vitro or in vivo methods to identify in vivo modulators or drugs, e.g., chemical or genetic modulators, to anergize T cells, by identifying an inhibitor of one of these CARD11 activities.

The invention provides methods for tolerizing a subject to an antigen (including, e.g., inducing humoral or cellular anergy to an immunogen). The method comprises providing an inhibitor of a CARD11 activity (an NFkB activating activity), e.g., a nucleic acid (e.g., antisense, ribozyme) or a polypeptide (e.g., antibody or dominant negative) of the invention. The inhibitor is administered in sufficient amounts to the subject to inhibit the expression of a CARD11 polypeptide. This generates a T cell defect comprising a defective co-signal up-regulation of CD69 through CD28 signaling after TCR stimulation to generating a toleragenic signal in the subject. The antigen is then administered to the subject. This tolerizes the subject to the antigen. Methods and compositions for tolerizing subjects to antigens known in the art can be adapted to practice the methods of this invention, see, e.g., U.S. Pat. Nos. 6,245,752; 6,211,160; 6,060,056; 5,935,577; 5,856,446; 5,833,990; 4,428,965.

Inbred Mouse Strains

The invention provides an inbred mouse and an inbred mouse strain that can be generated as described herein and bred by standard techniques, see, e.g., U.S. Pat. Nos. 6,040,495; 5,552,287.

In order to screen for mutations with recessive effects a number of strategies can be used, all involving a further two generations. For example, male G1 mice can be bred to wild-type female mice. The resulting progeny (G2 mice) can be interbred or bred back to the G1 father. The G3 mice that result from these crosses will be homozygotes for mutations in a small number of genes (3-6) in the genome, but the identity of these genes is unknown. With enough G3 mice, a good sampling of the genome should be present.

Peptides and Polypeptides

The invention provides isolated or recombinant polypeptides comprising an amino acid sequence having at least 95%, 96%, 97%, 98%, 99% or more sequence identity to SEQ ID NO:2 or SEQ ID NO:5 over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. In one aspect, the polypeptide comprises a sequence as set forth in SEQ ID NO:2, SEQ ID NO:5. The invention provides methods for inhibiting the activity of a CARD 11 polypeptide, e.g., a polypeptide of the invention. The invention also provides methods for screening for compositions that inhibit the activity of, or bind to (e.g., bind to the active site), of a CARD 11 polypeptide, e.g., a polypeptide of the invention.

In one aspect, the invention provides CARD11 polypeptides (and the nucleic acids encoding them) where one, some or all of the CARD11 heptad repeat leucines are replaces by another amino acid, e.g., substituted with a polar amino acid residue, e.g., substituted with a polar glutamine residue. In one aspect, the invention provides methods to disrupt the interaction of CARD11 (Carma-1) with other CC domain proteins, such as the B cell adaptor protein BCAP or the related molecule BANK, or methods to disrupt self-assembly of CARD11 (Carma-1) into higher order structures.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a CARD11 activity and for agents that can ameliorate a dermatitis, a B cell defect, a T cell defect, or a lymphoma. Polypeptides (e.g., antibody or dominant negative) of the invention can also be used to tolerize a subject to an antigen for, e.g., inducing humoral or cellular anergy to an immunogen.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, PA. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has an NFkB activating activity. A mimetic composition can also be within the scope of the invention if it can inhibit an activity of a CARD11 polypeptide of the invention, e.g., be a dominant negative mutant or, bind to an antibody of the invention.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form.

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a CARD11 polypeptide of the invention, resulting in modification of the structure of the polypeptide, without significantly altering its biological activity. For example, amino- or carboxyl-terminal, or internal, amino acids which are not required for NFkB activating activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

The terms “polypeptide” and “protein” as used herein, refer to amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

An exemplary CARD11 is presented below; SEQ ID NO:1 being the nucleic acid sequence, and SEQ ID NO:2 being the amino acid translation thereof:

| | | |
|---|---|---|
| 1 | gaggagggccagct | (SEQ ID NO:1) |
| 15 | atggatgactacatggagacgctgaaggatgaagaggaggcccta | |
| | M D D Y M E T L K D E E E A L | (SEQ ID NO:2) |
| 60 | tgggataacgtggaatgcaaccggcacatgctgagccgttacatc | |
| | W D N V E C N R H R H M L S R Y I | |
| 105 | aaccccgccaagctcaccccctacctgcgccagtgcaaggtcatc | |
| | N P A K L T P Y L R Q C K V I | |
| 150 | gatgagcaagatgaagacgaggtgctcaatgcgcccatgctgccg | |
| | D E Q D E D E V L N A P M L P | |
| 195 | tccaagatcaaccgtgcaggccgattgttggacattcttcacacc | |
| | S K I N R A G R L L D I L H T | |
| 240 | aagggacaaaggggctatgtggtcttcctggagagcctggagttt | |
| | K G Q R G Y V V F L E S L E F | |
| 285 | tactacccagaactttacaaactggtgactggaaaggaacccacc | |
| | Y Y P E L Y K L V T G K E P T | |
| 330 | cggagattctccaccattgtggtggaggaaggccatgagggcctc | |
| | R R F S T I V V E E G H E G L | |
| 375 | acacacttcctgatgaacgaggtcatcaaactgcagcagcaagtg | |
| | T H F L M N E V I K L Q Q G V | |
| 420 | aaagccaaggaccttcagcgctgtgagctgctggccaagtcccgg | |
| | K A K D L Q R C E L L A K S R | |
| 465 | caactggaggatgagaagaagcagctgagcctgatacgggtggag | |
| | Q L E D E K K Q L S L I R V E | |
| 510 | ctgctgaccttccaggagcgatactacaagatgaaggaggagcgg | |
| | L L T F Q E R Y Y K M K E E R | |
| 555 | gacagctacaatgacgagctcgtcaaggtcaaggacgacaactac | |
| | D S Y N D E L V K V K D D N Y | |
| 600 | aacttagccatgcgctacgcccagctcagtgaggagaaaaacatg | |
| | N L A M R Y A Q L S E E K N M | |
| 645 | gcggtgatgaggagccgcgacctccaactcgagatcgaccagctc | |
| | A V M R S R D L Q L E I D Q L | |
| 690 | aaacaccgactgaacaagatggaggaggaatgcaagctggagaga | |
| | K H R L N K M E E E C K L E R | |
| 735 | aatcagtccctcaagctcaagaatgacatcgagaaccggcccagg | |
| | N Q S L K L K N D I E N R P R | |
| 780 | aaggagcaggtcctggagctggagcgggagaatgagatgctgaag | |
| | K E Q V L E L E R E N E M L K | |
| 825 | acgaaaattcaggagctgcagtccatcatccaggctggcaagcgc | |
| | T K I Q E L Q S I I Q A G K R | |
| 870 | agcctccctgactcagacaaggccatcttggacatcctggaacat | |
| | S L P D S D K A I L D I L E H | |
| 915 | gaccggaaggaggcgctagaggaccggcaggaactggtcaacaaa | |
| | D R K E A L E D R Q E L V N K | |
| 960 | atttacaacctacaagaggaagtccgccaggcggaggagctgcgg | |
| | I Y N L Q E E V R Q A E E L R | |
| 1005 | gataagtacctggaggagaaggaagacctggaactcaagtgttca | |
| | D K Y L E E K E D L E L K C S | |
| 1050 | accctggggaaggactgtgaaatgtacaagcaccgcatgaacaca | |
| | T L G K D C E M Y K H R M N T | |
| 1095 | gttatgctgcagctggaggaggtggagcgggagcgggaccaggcc | |
| | V M L Q L E E V E R E R D Q A | |
| 1140 | ttccactcccgagatgaggcacagacacagtactcacagtgctta | |
| | F H S R D S A Q T Q Y S G C L | |
| 1185 | atcgagaaggacaagtaccggaagcagatccgggagctggaggag | |
| | I E K D K Y R K Q I R E L E E | |
| 1230 | aagaacgatgagatgcgtattgagatggtgaggagggaggcctgt | |
| | K N D E M R I E M V R R E A C | |
| 1275 | attgtcaacctggaaagcaagctccggcgcctgtccaaggacaac | |
| | I V N L S S K L R R L S K D N | |
| 1320 | ggcagcctcgaccagagtctgcctagacaccttccagccaccatc | |
| | G S L D Q S L P R H L P A T I | |
| 1365 | atctcacagaaccttggagacaccagccccaggaccaatggccag | |
| | I S G N L G D T S P R T N G Q | |
| 1410 | gaagctgatgattcttcaacctcagaagagtctcccgaagacagc | |
| | S A D D S S T S E E S P E D S | |
| 1455 | aagtactttctgccttaccacccaccccggcgccggatgaaccta | |
| | K Y F L P Y H P P R R R M N L | |
| 1500 | aagggcatccagctgcagagagccaaatcccccatcagcatgaag | |
| | K G I Q L Q R A K S P I S M K | |
| 1545 | caagcatctgagtttcaagtcaaggggcacgaagaggatttcaca | |
| | Q A S S F Q V K G H E S D F T | |
| 1590 | gacggcagccccagttcctcccgctcgctgcctgtcaccagctct | |
| | D G S P S S S R S L P V T S S | |
| 1635 | ttctccaagatgcaaccccatcggagccgcagcagcatcatgtca | |
| | F S K M Q P H R S R S S I M S | |
| 1680 | atcacggcagagcccccgggaaatgactccatagtcagacgctgt | |
| | I T A E P P G N D S I V R R C | |
| 1725 | aaggaagatgcgccacaccggagcacggtggaagaagacaacgat | |
| | K E D A P H R S T V E E D N D | |
| 1770 | agctgtgggtttgatgccttagaccttgacgatgaaaatcacgaa | |
| | S C G F D A L D L D D E N H E | |
| 1815 | cgttattcctttggacctccctccatccactcctcctcctcttca | |
| | R Y S F G P P S I H S S S S S | |
| 1860 | caccagtcagagggactggatgcctacgacctggagcaggtcaac | |
| | H Q S E G L D A Y D L E Q V N | |
| 1905 | ctcatgttacgaaagttctctttggaaaggcccttccggccatcg | |
| | L M L R K F S L E R P F R P S | |
| 1950 | gtcacatctgggggtcacgtgcggggcaccgggcccttggtccag | |
| | V T S G G H V R G T G P L V Q | |
| 1995 | cacacaactctgaatggcgatgggctcatcacgcagctcaccctt | |
| | H T T L N G D G L I T Q L T L | |
| 2040 | ctgggcggcaatgcacgcgggagcttcattcactctgtcaagcca | |
| | L G G N A R G S F I H S V K P | |
| 2085 | ggctcactggctgagagggccggactgcgtgagggccaccaactc | |
| | G S L A E R A G L R E G H Q L | |
| 2130 | ctgctgctggaaggttgcatccgaggcgaaaggcagagcgttcca | |
| | L L L E G C I R Q E R Q S V P | |
| 2175 | ctggatgcgtgcacaaaagaagaggcccgttggaccatccagagg | |
| | L D A C T K E E A R W T I Q R | |
| 2220 | tgcagtggcctcatcactctgcattacaaggtcaaccatgaagga | |
| | C S G L I T L H Y K V N H E G | |
| 2265 | taccggaagctgctgaaggagatggaggatggtctgatcacatca | |
| | Y R K L L K E M E D G L I T S | |
| 2310 | ggggactcgttctatatccgcctgaacctgaacatctccagccag | |
| | G D S F Y I R L N L N I S S Q | |
| 2355 | ctggatgcctgctccatgtccctcaagtgtgacgacgtggtgcat | |
| | L D A C S M S L K C D D V V H | |
| 2400 | gtcctagacaccatgtaccaggacaggcacgagtggctgtgtgca | |
| | V L D T M Y Q D R H E W L C A | |
| 2445 | cgagtcgaccccttcactgaccaagacctggacacgggcaccatc | |
| | R V D P F T D Q D L D T G T I | |
| 2490 | cccagctacagccgggctcaacagcttctcctggtgaagctccag | |
| | P S Y S R A Q Q L L L V K L Q | |
| 2535 | cggttggttcacagaggcaaccgggaagaggcagacagcgctcac | |
| | R L V H R G N R E E A D S A H | |
| 2580 | cacaccctgcgcagcctccggaacaccctgcagcccgaagagatg | |
| | H T L R S L R N T L Q P E E M | |
| 2625 | ctttcgacgagcgacccccgagtcagcccccgcctctccagagcg | |
| | L S T S D P R V S P R L S R A | |
| 2670 | agtttcttctttggccagctcctgcagtttgtcagccggtcagaa | |
| | S F F F G Q L L Q F V S R S E | |
| 2715 | aacaagtacaaaagaatgaacagcaatgagcgcgtgagaatcatc | |
| | N K Y K R M N S N E R V R I I | |
| 2760 | tctgggagtcccctggggagcctctcccggtcctcgctggatgcc | |
| | S G S P L G S L S R S S L D A | |
| 2805 | accaaactcctgaccgagaagcatgaagaactggatcctgagaat | |
| | T K L L T E K H E E L D P E N | |
| 2850 | gagctcagccggaacctcaccctgatcccttacagcctggtgcgc | |
| | E L S R N L T L I P Y S L V R | |
| 2895 | gctttccactgtgagcgccgcaggcctgtgctcttcacgcccacc | |
| | A F H C E R R R P V L F T P T | |
| 2940 | atgctggccaagacattggtgcagaagctgctcaactcagggggt | |
| | M L A K T L V Q K L L N S G G | |
| 2985 | gccatggagttcaccatctgcaagtcagatattgtcacaagagat | |
| | A M E F T I C K S D I V T R D | |
| 3030 | gagttcctccgaaagcagaagacagagaccatcatctactcccgg | |
| | E F L R K Q K T E T I I Y S R | |
| 3075 | gaaaagaaccccaacacctttgaatgcatcgtccctgccaacatt | |
| | E K N P N T F E C I V P A N I | |
| 3120 | gaggctgtggcagccaagaacaaacactgcctgctggaggctggg | |
| | E A V A A K N K H C L L E A G | |
| 3165 | atcggctgtgtgcgcgacctgatcaagtgcaaggtgtaccccata | |
| | I G C V R D L I K C K V Y P I | |
| 3210 | gtgctgctcatccgggtgagcgagaagaacatcaaacggttcagg | |
| | V L L I R V S E K N I K R F R | |
| 3255 | aagctgctgccgcggccagagacggaagaggaattcctgcgagtg | |
| | K L L P R P E T E E E F L R V | |
| 3300 | tgcaggctcaaagagaaggagctggaggcgctgccctgcctctac | |
| | C R L K E K E L E A L P C L Y | |
| 3345 | gccaccgtggaagctgagatgtggagcagcgtggaggagctgctg | |
| | A T V E A E M W S S V E E L L | |
| 3390 | cgagtcctcaaagacaagattgtagaggagcagcgcaagaccatc | |
| | R V L K D K I V E E Q R K T I | |
| 3435 | tgggtggacgaggaccagctgtgagcttgtctgggtctgacctac | |
| | W V D E D Q L * | |
| 3480 | acacagacacaccgg 3494 | |

An exemplary CARD11 variant is presented below; SEQ ID NO:4 being the nucleic acid sequence, and SEQ ID NO:5 being the amino acid translation thereof. Compared to SEQ ID NO:2 (the functional, wild type mouse CARD11) it has a mutation at base 907, amino acid L←Q (leucine to glutamine), that inactivates its activity.

| | | |
|---|---|---|
| 1 | gaggagggccagct | (SEQ ID NO:4) |
| 15 | atggatgactacatggagacgctgaaggatgaagaggaggcccta | |
| | M D D Y M E T L K D E E E A L | (SEQ ID NO:5) |
| 60 | tgggataacgtggaatgcaaccggcacatgctgagccgttacatc | |
| | W D N V E C N R H M L S R Y I | |
| 105 | aaccccgccaagctcaccccctacctgcgccagtgcaaggtcatc | |
| | N P A K L T P Y L R Q C K V I | |
| 150 | gatgagcaagatgaagacgaggtgctcaatgcgcccatgctgccg | |
| | D E Q D E D E V L N A P M L P | |
| 195 | tccaagatcaaccgtgcaggccgattgttggacattcttcacacc | |
| | S K I N R A G R L L D I L H T | |
| 240 | aagggacaaaggggctatgtggtcttcctggagagcctggagttt | |
| | K G Q R G Y V V F L E S L E F | |
| 285 | tactacccagaactttacaaactggtgactggaaaggaacccacc | |
| | Y Y P E L Y K L V T G K E P T | |
| 330 | cggagattctccaccattgtggtggaggaaggccatgagggcctc | |
| | R R F S T I V V E E G H E G L | |
| 375 | acacacttcctgatgaacgaggtcatcaaactgcagcagcaagtg | |
| | T H F L M N E V I K L Q Q Q V | |
| 420 | aaagccaaggaccttcagcgctgtgagctgctggccaagtcccgg | |
| | K A K D L Q R C E L L A K S R | |
| 465 | caactggaggatgagaagaagcagctgagcctgataCgggtggag | |
| | Q L E D E K K Q L S L I R V E | |
| 510 | ctgctgaccttccaggagcgatactacaagatgaaggaggagcgg | |
| | L L T F Q E R Y Y K M K E E R | |
| 555 | gacagctacaatgacgagctcgtcaaggtcaaggacgacaactac | |
| | D S Y N D E L V K V K D D N Y | |
| 600 | aacttagccatgcgctacgcccagctcagtgaggagaaaaacatg | |
| | N L A M R Y A Q L S E E K N M | |
| 645 | gcggtgatgaggagccgcgacctccaactcgagatcgaccagctc | |
| | A V M R S R D L Q L E I D Q L | |
| 690 | aaacaccgactgaacaagatggaggaggaatgcaagctggagaga | |
| | K H R L N K M E E E C K L E R | |
| 735 | aatcagtccctcaagctcaagaatgacatcgagaaccggcccagg | |
| | N Q S L K L K N D I E N R P R | |
| 780 | aaggagcaggtcctggagctggagcgggagaatgagatgctgaag | |
| | K E Q V L E L E R E N E M L K | |
| 825 | acgaaaattcaggagctgcagtccatcatccaggctggcaagcgc | |
| | T K I Q E L Q S I I Q A G K R | |
| 870 | agcctccctgactcagacaaggccatcttggacatccaggaacat | |
| | S L P D S D K A I L D I Q E H | |
| 915 | gaccggaaggaggcgctagaggaccggcaggaactggtcaacaaa | |
| | D R K E A L E D R Q E L V N K | |
| 960 | atttacaacctacaagaggaagtccgccaggcggaggagctgcgg | |
| | I Y N L Q E E V R Q A E E L R | |
| 1005 | gataagtacctggaggagaaggaagacctggaactcaagtgttca | |
| | D K Y L E E K E D L E L K C S | |
| 1050 | accctggggaaggactgtgaaatgtacaagcaccgcatgaacaca | |
| | T L G K D C E M Y K H R M N T | |
| 1095 | gttatgctgcagctggaggaggtggagcgggagcgggaccaggcc | |
| | V M L Q L E E V E R E R D Q A | |
| 1140 | ttccactcccgagatgaggcacagacacagtactcac.agtgctta | |
| | F H S R D E A Q T Q Y S Q C L | |
| 1185 | atcgagaaggacaagtaccggaagcagatccgggagctggaggag | |
| | I E K D K Y R K Q I R E L E E | |
| 1230 | aagaacgatgagatgcgtattgagatggtgaggagggaggcctgt | |
| | K N D E M R I E M V R R E A C | |
| 1275 | attgtcaacctggaaagcaagctccggcgcctgtccaaggacaac | |
| | I V N L E S K L R R L S K D N | |
| 1320 | ggcagcctcgaccagagtctgcctagacaccttccagccaccatc | |
| | G S L D Q S L P R H L P A T I | |
| 1365 | atctcacagaaccttggagacaccagccccaggaccaatggccag | |
| | I S Q N L G D T S P R T N G Q | |
| 1410 | gaagctgatgattcttcaacctcagaagagtctcccgaagacagc | |
| | E A D D S S T S E E S P E D S | |
| 1455 | aagtactttctgccttaccacccaccccggcgccggatgaaccta | |
| | K Y F L P Y H P P R R R M N L | |
| 1500 | aagggcatccagctgcagagagccaaatcccccatcagcatgaag | |
| | K G I Q L Q R A K S P I S M K | |
| 1545 | caagcatctgagtttcaagtcaaggggcacgaagaggatttcaca | |
| | Q A S E F Q V K G H E E D F T | |
| 1590 | gacggcagccccagttcctcccgctcgctgcctgtcaccagctct | |
| | D G S P S S S R S L P V T S S | |
| 1635 | ttctccaagatgcaaccccatcggagccgcagcagcatcatgtca | |
| | F S K M Q P H R S R S S I M S | |
| 1680 | atcacggcagagcccccgggaaatgactccatagtcagacgctgt | |
| | I T A E P P G N D S I V R R C | |
| 1725 | aaggaagatgcgccacaccggagcacggtggaagaagacaacgat | |
| | K E D A P H R S T V E E D N D | |
| 1770 | agctgtgggtttgatgccttagaccttgacgatgaaaatcacgaa | |
| | S C G F D A L D L D D E N H E | |
| 1815 | cgttattcctttggacctccctccatccactcctcctcctcttca | |
| | R Y S F G P P S I H S S S S S | |
| 1860 | caccagtcagagggactggatgcctacgacctggagcaggtcaac | |
| | H Q S E G L D A Y D L E Q V N | |
| 1905 | ctcatgttacgaaagttctctttggaaaggcccttccggccatcg | |
| | L M L R K F S L E R P F R P S | |
| 1950 | gtcacatctgggggtcacgtgcggggcaccgggcccttggtccag | |
| | V T S G G H V R G T G P L V Q | |
| 1995 | cacacaactctgaatggcgatgggctcatcacgcagctcaccctt | |
| | H T T L N G D G L I T Q L T L | |
| 2040 | ctgggcggcaatgcacgcgggagcttcattcactctgtcaagcca | |
| | L G G N A R G S F I H S V K P | |
| 2085 | ggctcactggctgagagggccggactgcgtgagggccaccaactc | |
| | G S L A E R A G L R E G H Q L | |
| 2130 | ctgctgctggaaggttgcatccgaggcgaaaggcagagcgttcca | |
| | L L L E G C I R G E R Q S V P | |
| 2175 | ctggatgcgtgcacaaaagaagaggcccgttggaccatccagagg | |
| | L D A C T K E E A R W T I Q R | |
| 2220 | tgcagtggcctcatcactctgcattacaaggtcaaccatgaagga | |
| | C S G L I T L H Y K V N H E G | |
| 2265 | taccggaagctgctgaaggagatggaggatggtctgatcacatca | |
| | Y R K L L K E M E D G L I T S | |
| 2310 | ggggactcgttctatatccgcctgaacctgaacatctccagccag | |
| | G D S F Y I R L N L N I S S Q | |
| 2355 | ctggatgcctgctccatgtccctcaagtgtgacgacgtggtgcat | |
| | L D A C S M S L K C D D V V H | |
| 2400 | gtcctagacaccatgtaccaggacaggcacgagtggctgtgtgca | |
| | V L D T M Y Q D R H E W L C A | |
| 2445 | cgagtcgaccccttcactgaccaagacctggacacgggcaccatc | |
| | R V D P F T D Q D L D T G T I | |
| 2490 | cccagctacagccgggctcaacagcttctcctggtgaagctccag | |
| | P S Y S R A Q Q L L L V K L Q | |
| 2535 | cggttggttcacagaggcaaccgggaagaggcagacagcgctcac | |
| | R L V H R G N R E E A D S A H | |
| 2580 | cacaccctgcgcagcctccggaacaccctgcagcccgaagagatg | |
| | H T L R S L R N T L Q P E E M | |
| 2625 | ctttcgacgagcgacccccgagtcagcccccgcctctccagagcg | |
| | L S T S D P R V S P R L S R A | |
| 2670 | agtttcttctttggccagctcctgcagtttgtcagccggtcagaa | |
| | S F F F G Q L L Q F V S R S E | |
| 2715 | aacaagtacaaaagaatgaacagcaatgagcgcgtgagaatcatc | |
| | N K Y K R N N S N E R V R I I | |
| 2760 | tctgggagtcccctggggagcctctcccggtcctcgctggatgcc | |
| | S G S P L G S L S R S S L D A | |
| 2805 | accaaactcctgaccgagaagcatgaagaactggatcctgagaat | |
| | T K L L T E K H E E L D P E N | |
| 2850 | gagctcagccggaacctcaccctgatcccttacagcctggtgcgc | |
| | E L S R N L T L I P Y S L V R | |
| 2895 | gctttccactgtgagcgccgcaggcctgtgctcttcacgcccacc | |
| | A F H C E R R R P V L F T P T | |
| 2940 | atgctggccaagacattggtgcagaagctgctcaactcagggggt | |
| | M L A K T L V Q K L L N S G G | |
| 2985 | gccatggagttcaccatctgcaagtcagatattgtcacaagagat | |
| | A M E F T I C K S D I V T R D | |
| 3030 | gagttcctccgaaagcagaagacagagaccatcatctactcccgg | |
| | E F L R K Q K T E T I I Y S R | |
| 3075 | gaaaagaaccccaacacctttgaatgcatcgtccctgccaacatt | |
| | E K N P N T F E C I V P A N I | |
| 3120 | gaggctgtggcagccaagaacaaacactgcctgctggaggctggg | |
| | E A V A A K N K H C L L E A G | |
| 3165 | atcggctgtgtgcgcgacctgatcaagtgcaaggtgtaccccata | |
| | I G C V R D L I K C K V Y P I | |
| 3210 | gtgctgctcatccgggtgagcgagaagaacatcaaacggttcagg | |
| | V L L I R V S E K N I K R F R | |
| 3255 | aagctgctgccgcggccagagacggaagaggaattcctgcgagtg | |
| | K L L P R P E T E E E F L R V | |
| 3300 | tgcaggctcaaagagaaggagctggaggcgctgccctgcctctac | |
| | C R L K E K E L E A L P C L Y | |
| 3345 | gccaccgtggaagctgagatgtggagcagcgtggaggagctgctg | |
| | A T V E A E M W S S V E E L L | |
| 3390 | cgagtcctcaaagacaagattgtagaggagcagcgcaagaccatc | |
| | R V L K D K I V E E Q R K T I | |
| 3435 | tgggtggacgaggaccagctgtgagcttgtctgggtctgacctac | |
| | W V D E D Q L * | |
| 3480 | acacagacacaccgg 3494 | |

Screening Methodologies

In practicing the methods of the invention, a variety of apparatus and methodologies can be used to in conjunction with the polypeptides and nucleic acids of the invention, e.g., to screen polypeptides for NFkB activating activity, to screen compounds as potential modulators (e.g., inhibitors or activators) of a CARD11 activity, e.g., an NFkB activating activity, for antibodies that bind to a polypeptide of the invention, for nucleic acids that hybridize to a nucleic acid of the invention, to screen for cells expressing a polypeptide of the invention and the like.

In one aspect, the peptides and polypeptides of the invention can be bound to a solid support. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of peptides to a solid support can be direct (i.e., the protein contacts the solid support) or indirect (a particular compound or compounds are bound to the support and the target protein binds to this compound rather than the solid support). Peptides can be immobilized either covalently (e.g., utilizing single reactive thiol groups of cysteine residues (see, e.g., Colliuod (1993) Bioconjugate Chem. 4:528-536) or non-covalently but specifically (e.g., via immobilized antibodies (see, e.g., Schuhmann (1991) Adv. Mater. 3:388-391; Lu (1995) Anal. Chem. 67:83-87; the biotin/strepavidin system (see, e.g., Iwane (1997) Biophys. Biochem. Res. Comm. 230:76-80); metal chelating, e.g., Langmuir-Blodgett films (see, e.g., Ng (1995) Langmuir 11:4048-55); metal-chelating self-assembled monolayers (see, e.g., Sigal (1996) Anal. Chem. 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SLAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377:525-531 (1989).

Arrays or “Biochips”

The invention provides methods for identifying/screening for modulators (e.g., inhibitors, activators) of a CARD11 activity, e.g., an NFkB activating activity, using arrays. Potential modulators, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention, e.g., a CARD11 activity. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays can be used to simultaneously quantify a plurality of proteins. Small molecule arrays can be used to simultaneously analyze a plurality of CARD11 modulating or binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

Combinatorial Chemical Libraries

The invention provides methods for identifying/screening for modulators (e.g., inhibitors, activators) of a CARD11 activity, e.g., an NFkB activating activity. In practicing the screening methods of the invention, a test compound is provided. It can be contacted with a polypeptide of the invention in vitro or administered to a cell of the invention or an animal of the invention in vivo. Compounds are also screened using the compositions, cells, non-human animals and methods of the invention for their ability to ameliorate dermatitis, B cell defects and/or T cell defects, or a lymphoma, and, or for their ability to generate a toleragenic environment in an animal. Combinatorial chemical libraries are one means to assist in the generation of new chemical compound leads for, e.g., compounds that inhibit an NFkB activating activity or, using a transgenic or a knockout non-human animal of the invention, a compound that can be used to treat or ameliorate a dermatitis, a B cell defect, a T cell defect, a lymphoma, or to be used to tolerize a subject to an antigen.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250). Preparation and screening of combinatorial chemical libraries are well known to those of skill in the art, see, e.g., U.S. Pat. No. 6,004,617; 5,985,356. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), non-peptidal peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, PA, Martek Biosciences, Columbia, Md., etc.).

Antibodies and Antibody-Based Screening Methods

The invention provides isolated or recombinant antibodies that specifically bind to a polypeptide or nucleic acid of the invention. These antibodies can be used to isolate, identify or quantify a polypeptide of the invention or related polypeptides. These antibodies can be used to isolate other polypeptides within the scope the invention or other related NFkB activating polypeptides.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies which bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides from other organisms and samples. In such techniques, polypeptides from the organism are contacted with the antibody and those polypeptides which specifically bind the antibody are detected. Any of the procedures described above may be used to detect antibody binding.

Kits

The invention provides kits comprising the compositions, e.g., nucleic acids, expression cassettes, vectors, cells, polypeptides (e.g., CARD11/NFkB activating activity polypeptides) and/or antibodies of the invention. The kits also can contain instructional material teaching the methodologies and uses of the invention, as described herein.

Therapeutic Applications

The compounds and modulators identified by the methods of the present invention may be used in a variety of methods of treatment. Thus, the present invention provides compositions and methods for treating an autoimmune disease or disorder or a lymphoma.

Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, pemiciousanemia, rapidly progressive glomerulonephritis and fibrosing alveolitis.

The uncontrolled growth of lymphoma cells can be driven through activation of B cell signaling pathways which include CARD11. In particular, activation of nuclear factor kappa B (NFkB) has been described for diffuse B cell lymphomas, mucosa-associated lymphoid tissue (MALT) lymphomas, Epstein Barr Virus (EBV)-associated lymphomas, multiple myeloma, and Hodgkin lymphomas. Furthermore, there is also evidence that supports a role for antigen signaling through the B cell antigen receptor (BCR or surface immunoglobulin) in driving the growth of lymphoma cells. As described herein, antigen driven activation of NFkB in B cells is dependent on CARD11. As many of the signaling pathways between normal and lymphoma B cells are shared, CARD11 is implicated in NFkB activation in lymphoma cells. Accordingly, inhibition of CARD11 activity provides a therapeutic approach for blocking lymphoma cell growth, as this intervention would cause a decrease in NFkB activity. Clinical success has been shown similarly in the treatment of multiple myeloma, where NFkB activation was blocked using an inhibitor of the proteasome (Velcade) causing complete and partial responses.

Formulation and Administration of Pharmaceutical Compositions

The invention provides pharmaceutical compositions comprising nucleic acids, peptides and polypeptides (including Abs) of the invention. As discussed above, the nucleic acids, peptides and polypeptides of the invention can be used to inhibit or activate expression of an endogenous CARD11 polypeptide. Such inhibition in a cell or a non-human animal can generate a screening modality for identifying compounds to treat or ameliorate a T cell defect, a B cell defects, a lymphoma and/or a dermatitis. Administration of a pharmaceutical composition of the invention to a subject is used to generate a toleragenic immunological environment in the subject. This can be used to tolerize the subject to an antigen.

The nucleic acids, peptides and polypeptides of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Nucleic acids, peptides or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani (1996) “Systemic delivery of peptides and proteins across absorptive mucosae” Crit. Rev. Ther. Drug Carrier Syst. 13:85-184. For topical, transdermal administration, the agents are formulated into ointments, creams, salves, powders and gels. Transdermal delivery systems can also include, e.g., patches.

The nucleic acids, peptides or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney (1998) Nat. Biotechnol. 16:153-157).

For inhalation, the nucleic acids, peptides or polypeptides of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the nucleic acid, peptides and/or polypeptides of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467, U.S. Pat. Nos. 4, 235,871, 4,501,728 and 4,837,028.

Treatment Regimens: Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton (1997) “Bioavailability and transport of peptides and peptide drugs into the brain” Peptides 18:1431-1439; Langer (1990) Science 249:1527-1533.

In therapeutic applications, compositions are administered to a patient suffering from a dermatitis, a lymphoma, B cell defect and/or a T cell defect in an amount sufficient to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The invention will be further described with reference to the following examples; however, it is to be understood that the invention is not limited to such examples.

### EXAMPLES

### Example 1: Generation and characterization of the inbred mouse strain of the invention

The invention provides a mouse strain with a point mutation in the mouse orthologue of CARD 11 (also called CARMA-1), causing selective defects in B and T cell responses that establish a central role for this membrane bound guanylate kinase (MAGUK) protein in coordinating the quality of antigen receptor signaling. The following example describes the generation and characterization of an inbred mouse strain of the invention.

In making the mice of the invention, animals were first mutagenized, then selected for a desired phenotype, and finally the gene responsible for the selected phenotype is identified and characterized. This approach is markedly different from the knockout companies, where a gene is targeted for mutation and then the phenotype of the resulting mouse is characterized. In summary, the approach of the instant invention is to generate interesting phenotypes first, then find the gene. In contrast, the knockout technique strategy can be summarized as starting with an interesting gene, then finding the phenotype.

A library of mice with random, genome wide point mutations was generated by treating C57BL/6 (i.e., B6) male mice with the chemical mutagen ethylnitrosurea (ENU). ENU treatment resulted in mutagenesis of spermatagonial stem cells, among other cells. Sperm that are descendants of these stem cells carry mutations in their genome as a result of the ENU treatment. It was estimated that the mutational rate is equivalent to loss of function mutations in about 50 genes of the genome.

Individual first generation male offspring were used as founders of inbreeding pedigrees by breeding with a wild-type (WT) female and then with several daughters. Each third generation offspring is thus expected to be homozygous for 12.5% of the mutations carried by the founder male.

G3 mice were grouped into pedigrees where mice of the same pedigree are derived from the same G1 grandparent. Each pedigree was estimated to contain 50 mutations in total, and 1 in 8 of the G3 mice will be homozygous recessive for a mutation in any one of these (for a G1*G2 cross). As there are about 30,000 genes in the genome, about 600 pedigrees are needed to cover the genome at lx.

About 20 G3 animals from each pedigree were screened for phenotypic variation. In the best case, multiple phenodeviants will be observed within a pedigree for a given trait. This pedigree then has a high probability of containing a mutation that affects that particular phenotype.

A mutant pedigree with the phenotype described below was identified using a mutational screen. On identification of the mutant pedigree, the mutation was maintained by breeding to B6 wild type mice. The mutation was mapped by outcrossing to another mouse strain. The offspring from this cross are intercrossed and the offspring were phenotyped and genotyped. The mice were backcrossed to B6 at least 3 times. An important point is that the mutation was on a B6 background to begin with, so the mice are genetically quite homogeneous. The only heterogeneity comes from other irrelevant ENU point mutations that might be segregating. However, statistically there will be few if any of these left in the strain that are functionally relevant, even after 1 or 2 backcrosses.

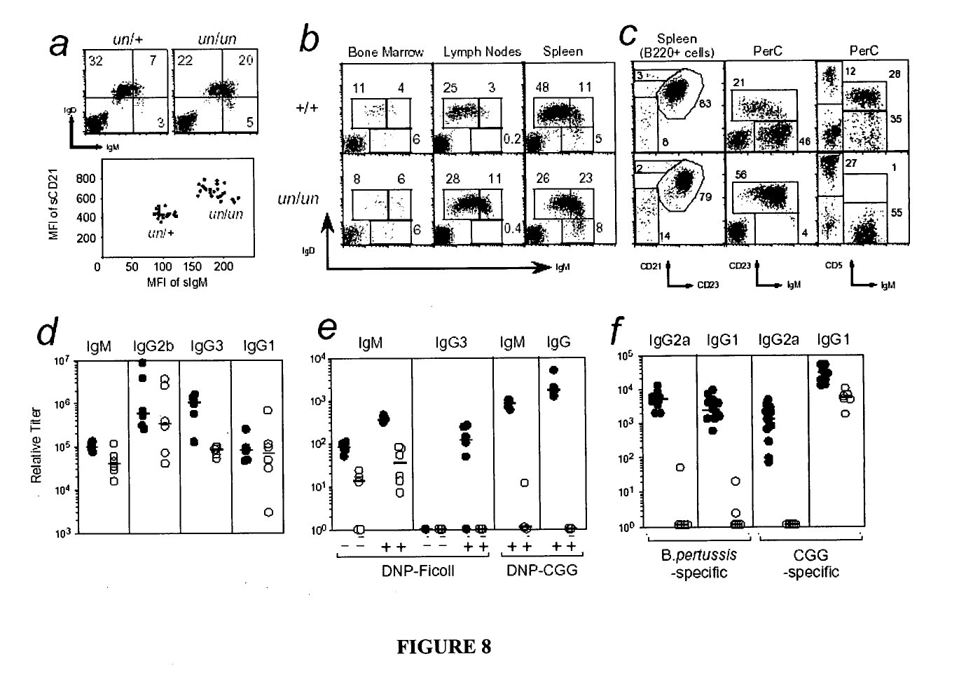

Approximately 20 third generation (G3) offspring in 185 pedigrees were screened for blood lymphocyte abnormalities by flow cytometry. Several mice in one pedigree exhibited an unusually high level of surface IgM antigen receptors on circulating IgD+ B cells (FIG. 8a). FIG. 8 illustrates data from experiments characterizing B cell populations and immune responses in unmodulated mice. In FIG. 8a peripheral blood cells were stained for surface IgM and IgD with percentage of cells in quadrants indicated. Lower panel shows mean fluorescence intensity (MFI) of CD21 and IgM of a group of homozygous (un/un) and heterozygous (un/+) unmodulated mice. FIG. 8b and FIG. 8c illustrate B cell sub-populations in lymphoid organs of wild-type and mutant mice. FIG. 8d illustrates basal serum antibody levels in wild-type ( ) and mutant (∘) mice. FIG. 8e illustrates the effect of DNP-specific antibodies in sera prior to immunization (−) and post-immunization (+). FIG. 8f illustrates CGG-specific and Bordetella pertussis-specific IgG1 and IgG2a antibody responses after combined immunization.

The unmodulated phenotype was inherited as a simple, fully penetrant recessive trait in progeny, and was characterized by 100% increased mean IgM and 50% higher expression of CD21 (FIG. 8a). Because surface IgM is normally modulated to lower levels on many mature recirculating B cells, the mutant strain was named “unmodulated”. This high surface IgM phenotype is reminiscent of previously described mutations in BCR signaling, notably in Bruton's tyrosine kinase (Btk), phospholipase C-gamma (PLCg), B cell activator of P13-kinase (BCAP), and vav1 and vav2.

In summary, the mutant pedigree strain of mice designated ‘unmodulated’ has the phenotype described below:

(I) Dermatitis. The inbred mice develop erythema of ears and weepy eyes at 10 weeks, progressing to dermatitis of ears and neck in >50% of mice at 4 months old. Infiltration of eosinophils is observed in inflamed areas of skin by histological evaluation. Circulating levels of IgE are elevated. Eosinophilia and hyper IgE are hallmarks of the human condition atopic dermatitis, suggesting that this mouse might be a good model for the human disease.

(II) Defects in Immunological Signaling.

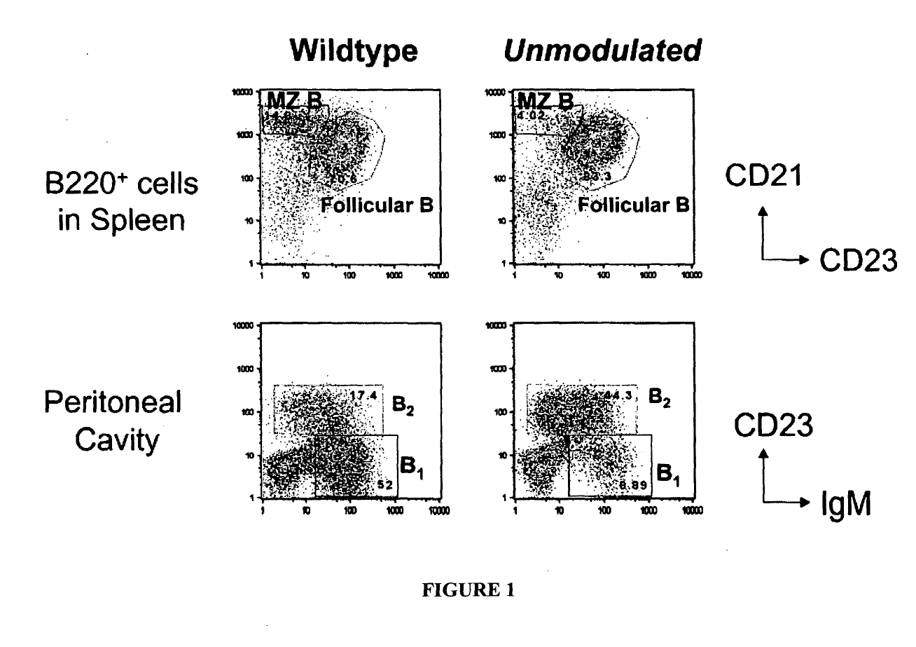

(a) Defects in B cell signaling. B cell development in the bone marrow appears normal, but the splenic follicular B cells arrest in their maturation and do not down-regulate surface IgM (hence ‘unmodulated’). In addition, marginal zone and peritoneal B cells are reduced in frequency, as illustrated by the summary of data in the two-dimensional two-color FACS, as shown in FIG. 1. Cells from the spleen and peritoneal cavity were stained with CD21 and CD23 and CD21 and IgM, respectively. These B cells are responsible for rapid secretion of relatively low affinity antibody in response to immune challenge. CD 21, CD23 and IgM are important B cell surface response regulators. CD19, through the CD19/CD21 complex, establishes a novel Src-family protein tyrosine kinase (PTK) amplification loop that regulates basal signaling thresholds and intensifies Src-family PTK activation following B cell antigen receptor (IgM) ligation. CD21 and CD23 are markers for activated B cells.

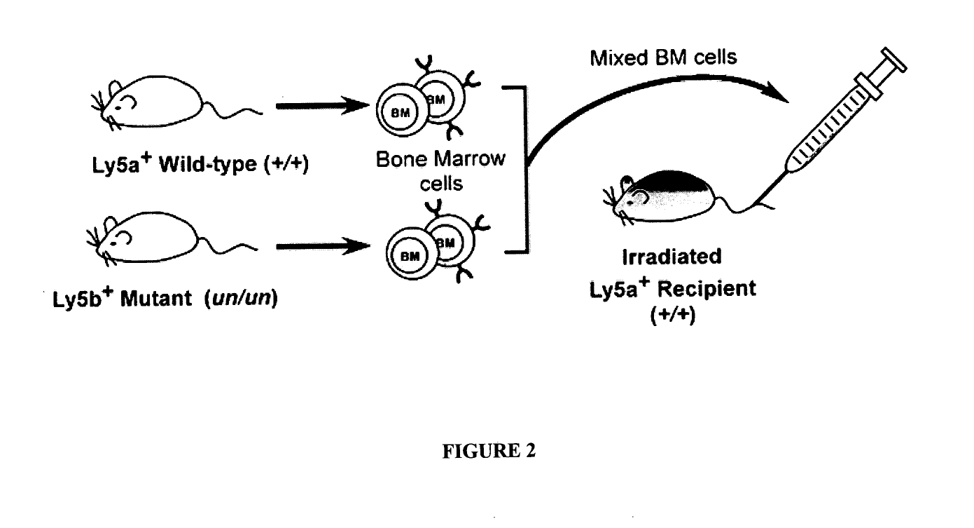

FIG. 2: A schematic of a mixed bone marrow chimera experiment.

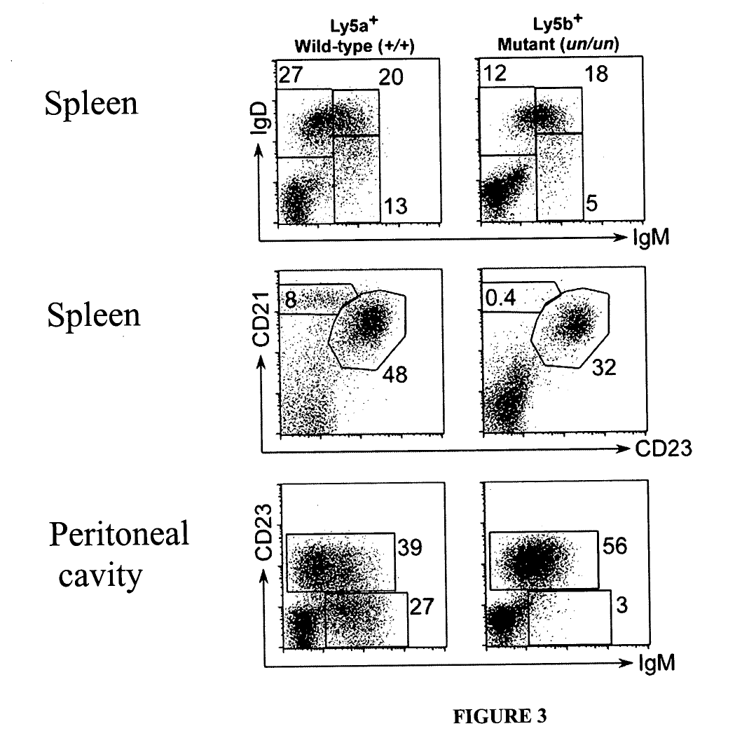

FIG. 3: A 2-D FACS showing “unmodulated” mice B cell phenotype is cell-intrinsic.

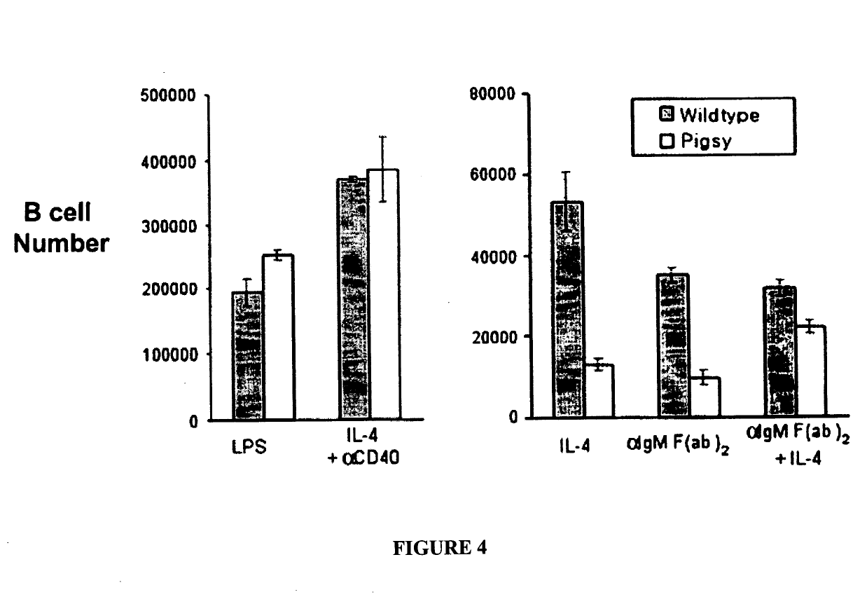

FIG. 4: “Unmodulated” mice B cells fail to respond to BCR crosslinking.

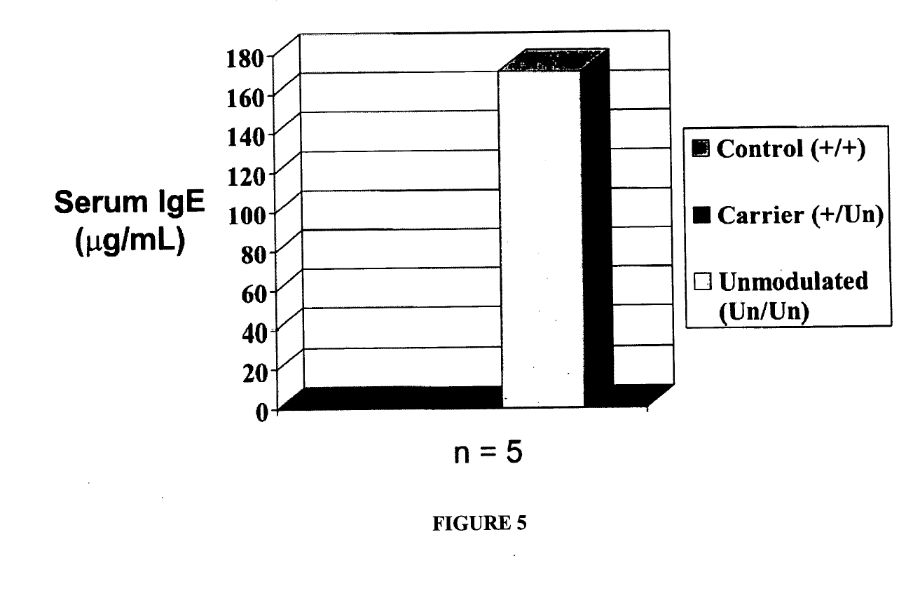

FIG. 5: “Unmodulated” mice with dermatitis have very high levels of serum IgE.

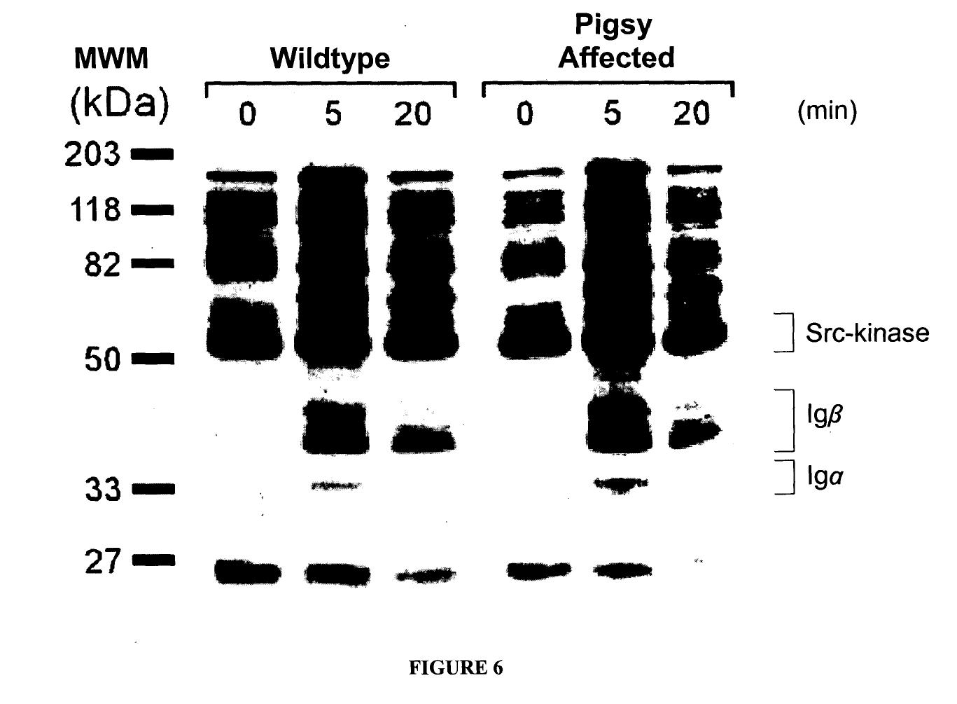

FIG. 6: Early signaling events in “Unmodulated” mice B cells are defective, as shown by Western blot with anti-pTyr(4G10) antibody.

This alteration in population frequencies of B cell subsets and antibody response is likely to be caused by a defect in signaling from the antigen receptor. B cells from these mice have a reduced mitogenic response to cross linking of the antigen receptor. Proximal signaling from antigen receptor is normal, as determined by anti-phosphotyrosine western blotting and calcium signaling. In addition, B cells have a reduced mitogenic response to a combination of PMA and ionomycin, suggesting that the defect in signaling in these cells is downstream of calcium and protein kinase C activation.

(b) Lymphocyte Development in Unmodulated Mice

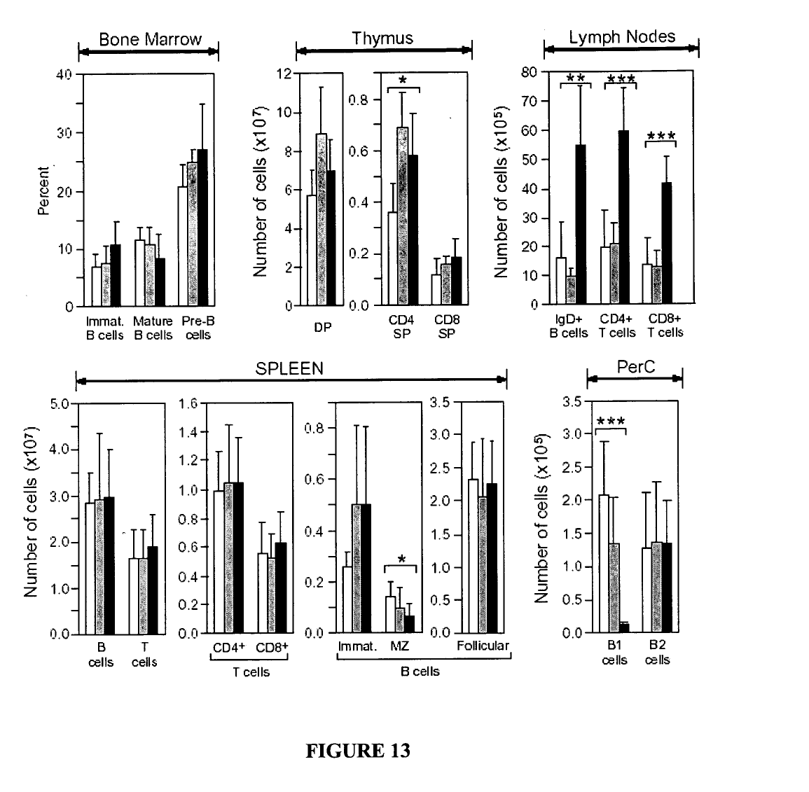

Lymphocyte development and peripheral subsets were analyzed in unmodulated mice by flow cytometry. In the bone marrow, no difference in frequency of pre-B cell or immature B cells could be detected between un/un homozygotes and +/+ or +/un controls, see FIG. 13.

FIG. 13 illustrates comparisons of primary and secondary lymphoid organ cellularity. Mice of 9-12 weeks of age and same sex were used for this analysis: wild-type (+/+, n=6, open bars), mutant heterozygotes (un/+, n=5, shaded bars) and mutant homozygotes (un/un, n=7, closed bars). Student t-test was applied to assess statistical significance of difference between wildtype control and mutant homozygote. *: P<0.05, **: P<0.01, ***: P<0.001

Each lymphocyte sub-population is determined as follows: Bone Marrow: Immature (IgM^{hi-intm}/B220^{intm}), Mature (IgM^{hi-intm}/B220^{hi}) and Pre-B cells (IgM^{lo}/B220^{intm}) Thymus: DP (CD4^{hi}/CD8^{hi}), CD4 SP (CD4^{hi}/CD8^{lo}) and CD8 SP (CD4^{lo}/CD8^{hi}). Lymph nodes: (pairs of inguinal, sub-axillary, brachial): IgD+ B cells (IgM^{hi-intm/hi}), CD4+ T cells (CD4^{hi}/CD8^{lo}) and CD8+ T cells (CD4^{lo}/CD8^{hi}). Spleen: B cells (B220^{hi}/Thy1.2^{lo}), T cells (B220^{lo}/Thy1.2^{hi}), CD4+ T cells (CD4^{hi}/CD8^{lo}), CD8+ T cells (CD4^{lo}/CD8^{hi}), Immature B cells (CD23^{lo}/CD21^{lo}/B220⁺), Marginal zone B cells (CD23^{lo}/CD21^{hi}/B220⁺) and Follicular B cells (CD23^{hi}/CD21^{intm}/B220⁺). Peritoneal cavity (PerC): B1 cells (IgM^{hi}/CD23^{lo}) and B2 cells (IgM^{hi-intm}/CD23^{hi}).

The numbers of immature T1 B cells and IgD+ mature follicular B cells in the spleen was also not different, see FIG. 13. The frequency of IgD+ cells in lymph nodes was normal, although there was an overall increase in lymph node B and T cell numbers, see FIG. 13. The chief difference in the IgD+ cells was a shift to higher levels of surface IgM. Marginal zone B cells in the spleen were reduced in number by 50% in unmodulated homozygotes, see FIG. 13. In the peritoneal cavity, there were normal numbers of CD23+follicular-type B cells, but an almost complete absence of the B1 cell subset, see FIG. 8c and FIG. 13. Consistent with the evidence linking the B1 cell subset to basal serum IgM secretion, serum from un/un mice had 50% less total IgM than heterozygous or wild-type control mice (p=0.01, see FIG. 8d). Serum IgG1 and IgG2b levels were not significantly different, but serum IgG3 levels were reduced in the mutant mice, see FIG. 8d. The number of CD4 single positive thymocytes was increased in the mutant mice, but CD8 single positives and CD4 CD8 double positive thymocytes were present in equal numbers between wild-type and mutant mice, see FIG. 13.

Construction of mixed bone marrow radiation chimeras reconstituted with 50:50 mixtures of B6-Ly5a wild-type and B6-un/un bone marrow confirmed that the CARD11 (Carmal) mutation does not affect B or T cell development or peripheral B or T cell numbers, and that the higher expression of IgM and absence of peritoneal B1 cells are autonomous to mutant B cells.

(c) Diminished Antibody Responses

Since the changes in surface IgM and absence of B1 cells in unmodulated mice resemble the phenotypic effects of BCR signaling mutations in the Btk/PLCg/vav pathway, whether there were also comparable deficits in B cell antibody responses to T cell independent type 2 antigen was tested.

Compared to B6 control mice, B6-un/un animals showed little increase in serum IgM and no increase in serum IgG titers to the DNP hapten following immunization with DNP-Ficoll, see FIG. 8e). There was also a dramatic decrease in the primary antibody response to immunization with a T-cell dependent antigen, DNP-chicken gamma globulin (CGG see FIG. 8e). When mice are immunized with a strongly immunogenic mixture of CGG and heat killed Bordetella pertussis organisms in alum, the serum response to CGG is normally skewed to the Th2-subtype of IgG1 whereas the antibodies to the bacterial antigen are primarily IgG2a associated with the Th1-type response see FIG. 8f. In un/un mice, the IgG1 response to CGG was lower but not absent, whereas there was no detectable IgG2a response to Pertussis organisms given at the same time.

(d) Selective B Cell Signaling and Response Deficits

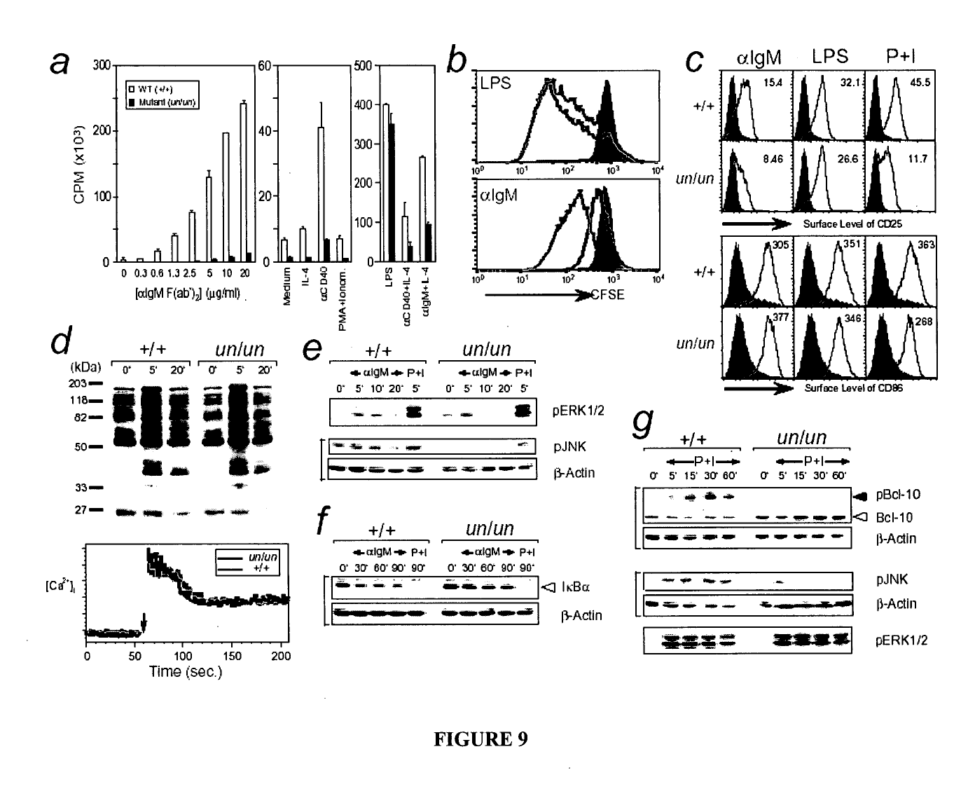

The defects in antibody response were further delineated by measuring unmodulated B cell responses to key B cell activating stimuli in vitro. Crosslinking B cell antigen receptors with antibodies to IgM stimulates vigorous cell division in wild-type B cells within 48 hours, but this response was greatly reduced in un/un B cells, see FIG. 9a. FIG. 9a illustrates DNA synthesis by splenic B cells cultured 48 hr with indicated stimuli. Mean+/−SD of triplicate samples is shown.

Splenocytes labeled with CFSE showed that mutant B cells were intrinsically poorer in survival and very few proceeded through more than one cell division by 72 hours, while wild-type B cells in the same culture divided extensively, see FIG. 9b). FIG. 9b illustrates the division of CFSE-labeled splenocytes from wildtype (grey) and unmodulated mice (black) stimulated for 72 hr. Shaded histograms show undivided cells in parallel cultures with IL-4 alone.

By contrast, the CARD11 (Carma-1) mutant B cells proliferated normally to bacterial lipopolysaccharide (LPS), demonstrating that the mutation selectively diminishes coupling of the BCR but not Toll-like receptor 4 to mitogenic signaling, see FIGS. 9a, 9b. Proliferation induced by antibody to the CD40 receptor, which mimics an important helper T cell signal, was also reduced although not as severely as the response to IgM crosslinking. Addition of the T cell-derived cytokine IL4 improved but did not fully restore proliferation to anti-IgM or anti-CD40, see FIG. 9a.

The initial phase of B cell activation is marked by upregulation of cell-cell interaction markers and cytokine receptors in response to different intracellular signals. The gene for the CD25 protein encodes the alpha subunit of the receptor for interleukin-2 and is induced through a pathway that requires signaling through the NFkB pathway. Relatively little CD25 was induced on unmodulated B cells when stimulated by B cell antigen receptor crosslinking, but this protein was induced normally when the mutant cells were stimulated by LPS, see FIG. 9c. FIG. 9c illustrates CD25 and CD86 levels on B cells after 16 hr stimulation. Shaded histograms represent unstimulated cells. Numbers indicate MFI.

A similar selective reduction in response to BCR but not TLR4 stimuli was observed for CD69 induction. BCR signaling and response nevertheless appeared normal as measured by induction of the T cell stimulating protein, CD86, see FIG. 9c. Stimulation with a combination of phorbol ester (PMA) and ionomycin to mimic downstream events in BCR signaling also elicited a diminished CD25 response in unmodulated B cells, see FIG. 9c. These results are consistent with a downstream and selective defect in B cell signaling.

Analysis of the immediate biochemical events in B cell antigen receptor signaling confirmed a distal, selective defect in B cells with mutant Carma-1. Crosslinking IgM receptors induced a normal pattern and intensity of tyrosine phosphorylation on the Iga/b receptor subunits and other cellular proteins, see FIG. 9d, and a normal amplitude and kinetics of intracellular calcium elevation, see FIG. 9d. FIG. 9d illustrates protein tyrosine phosphorylation after being stimulated with 10 μg/ml anti-IgM for times shown; lower panel: elevation of intracellular calcium, measured by Indo-1 fluorescence after stimulation with anti-IgM (arrow).

FIG. 9e illustrates phosphorylation of ERK and JNK measured in purified B cells by Western blotting with anti-phosphoERK1/2 or anti-phosphoJNK antibodies. Blots were re-probed for beta-actin to confirm equal loading.

Consistent with the normal intracellular calcium response, un/un cells exhibited a normal dephosphorylation and cytoplasmic/nuclear translocation of NFAT. The ERK map-kinase cascade was also activated normally by BCR crosslinking or the pharmacologic mimics PMA and ionomycin, see FIG. 9f. FIG. 9f illustrates BCR-induced degradation of IkB-alpha measured by Western blotting.

By contrast, BCR-induced degradation of the inhibitor of NfkB, IkBa, was diminished in un/un B cells, see FIG. 9g and BCR or PMA/ionomycin-induced phosphorylation of the JNK map-kinase cascade was reduced especially at later time-points after stimulation, see FIGS. 9f, 9g). FIG. 9g illustrates selective deficit in JNK and Bcl10 phosphorylation in purified B cells following stimulation with phorbol ester and ionomycin. Bcl10 was approximately 34 kDa, see unfilled arrowhead. PhosphoBcl10 was approximately 40 kDa, see filled arrowhead.

PMA and ionomycin induced a marked retardation in the electrophoretic mobility of Bcl-10 in wild-type B cells, consistent with the effect of phosphorylation shown by others. This response was undetectable in un/un B cells, see FIG. 9g. Thus, B cells with a CARD11 (Carma-1) mutation display a selective, partial deficit in antigen receptor coupling to the JNK and NfkB signaling pathways.

In summary, the B cell defect can be a defect in B cell development, an arrest in splenic follicular B cell maturation, an inability to down-regulate surface IgM, a reduction in frequency of marginal zone and peritoneal B cells, a defective T dependent and T independent antibody response, a reduced mitogenic response after cross linking of B cell antigen receptors or a reduced mitogenic response to a combination of PMA and ionomycin.

(e) Defects in Responsiveness of Unmodulated T Cells

Defects are also seen in T dependent and T independent antibody responses. The T cell defect includes a defective co-signal up-regulation of CD69 through CD28 signaling after T cell receptor (TCR) stimulation, a defective cell division response after CD28 signaling after T cell receptor (TCR) stimulation, a defective IL2 response after CD28 signaling after T cell receptor (TCR) stimulation and a defective IL2 receptor alpha chain response after CD28 signaling after T cell receptor (TCR) stimulation.

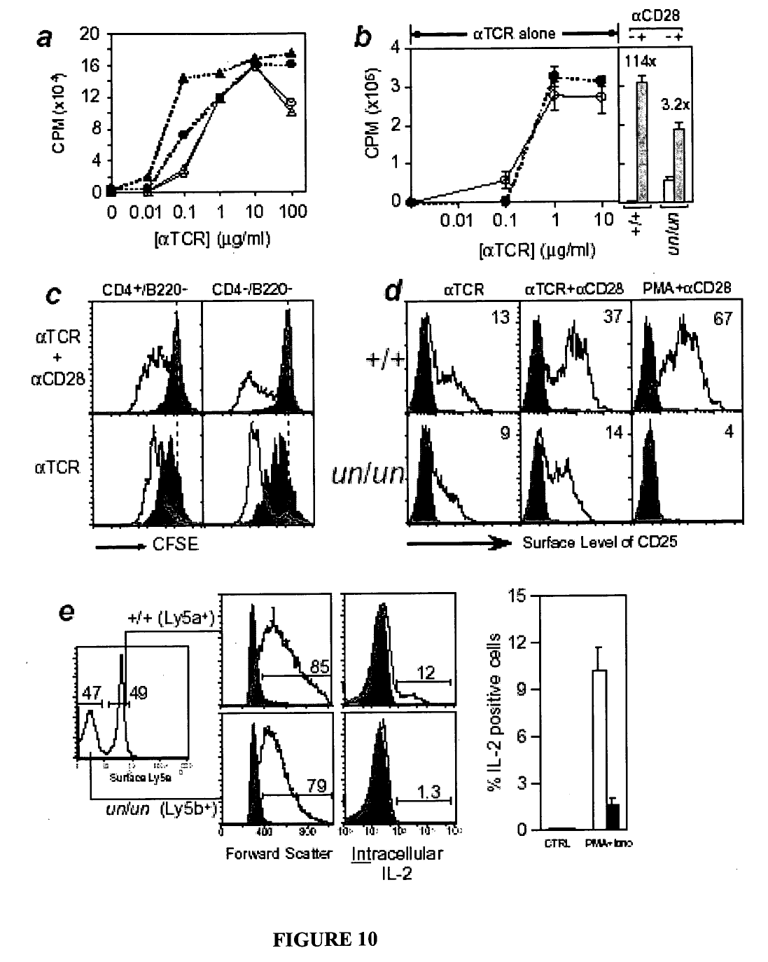

The poor antibody response of CARD11 (Carma-1) mutant mice to T cell dependent immunization, see, e.g., FIG. 8, suggested that T cell responses to antigen were also be affected by the mutation. To examine demonstrate that T cell responses to antigen were also be affected by the mutation, T cell activation responses were measured in vitro. Unmodulated T cells enlarged and proliferated almost normally during the first 48 hours of culture when stimulated by crosslinking their T cell antigen receptors, see FIG. 10a, FIG. 10b, although subsequent cell divisions were markedly reduced, see FIG. 10c. Proliferation, especially for successive cell divisions, is normally promoted by co-stimulation through CD28 receptors. The effects of CD28 co-stimulation were dramatically reduced in T cells with the mutant Carma-1, see FIGS. 10a, b. Co-culture experiments established that the proliferative defects through successive divisions are cell autonomous to T cells carrying the mutant Carma-1, see FIG. 10c. FIG. 10a illustrates DNA synthesis after 48 hr in splenocytes from wildtype (filled symbols) and mutant (unfilled symbols) mice stimulated with the indicated concentrations of plate-bound anti-TCR in the presence (circles) or absence (triangles) of anti-CD28. FIG. 10b illustrates DNA synthesis in purified splenic T cells stimulated with plate-bound anti-TCR alone, see the left panel, ●:+/+, ∘:un/un. The right panel shows proliferation to 0.1 μg/ml anti-TCR without or with anti-CD28 co-stimulation. Mean and SD are shown for three mice of each genotype. FIG. 10c illustrates divisions of CFSE-labeled wild-type (unshaded) and mutant (shaded) CD4 or CD8 splenocytes after 72 hr co-culture stimulated with low concentration of anti-TCR plus anti-CD28 or with saturating concentration of anti-TCR. Dashed lines indicate CFSE in undivided cells.

A similar selective defect in CD28 co-stimulation was apparent by measuring cell surface induction of CD25, see FIG. 10d. TCR stimulation alone elicited only low CD25 expression on a subset of wild-type or mutant T cells. Addition of agonistic antibody to CD28 greatly increased the frequency and levels of CD25 on wild-type T cells, but had only a modest effect on un/un T cells, see FIG. 10d. The capacity of CD28 to induce CD25 expression in combination with PMA was completely abolished in unmodulated T cells, see FIG. 10d. FIG. 10d illustrates induction of CD25 after 24 hr on CD4⁺ T cells stimulated as indicated (unshaded) or cultured without stimulation (shaded). Numbers represent MFI of CD25 on stimulated cells.

When cultured with PMA and Ionomycin to mimic potent TCR/CD28 co-stimulation, both mutant and wild-type T cells enlarged, but few of the mutant T cells produced measurable levels of IL-2, see FIG. 10e. Collectively, these findings indicate a relatively selective deficit in CD28 co-stimulation in T cells homozygous for the CARD11 (Carma-1) mutation. FIG. 10e illustrates blastogenesis and intracellular IL-2 in co-cultured wild-type and mutant splenocytes after 22 hr stimulation with phorbol ester and ionomycin. Shaded histograms, unstimulated cells; open histograms, stimulated cells. The mean percentage ±SD of cells secreting IL-2 in wild-type cells (open bars) is compared with that of mutant cells (closed bars).

(f) Spontaneous Atopy

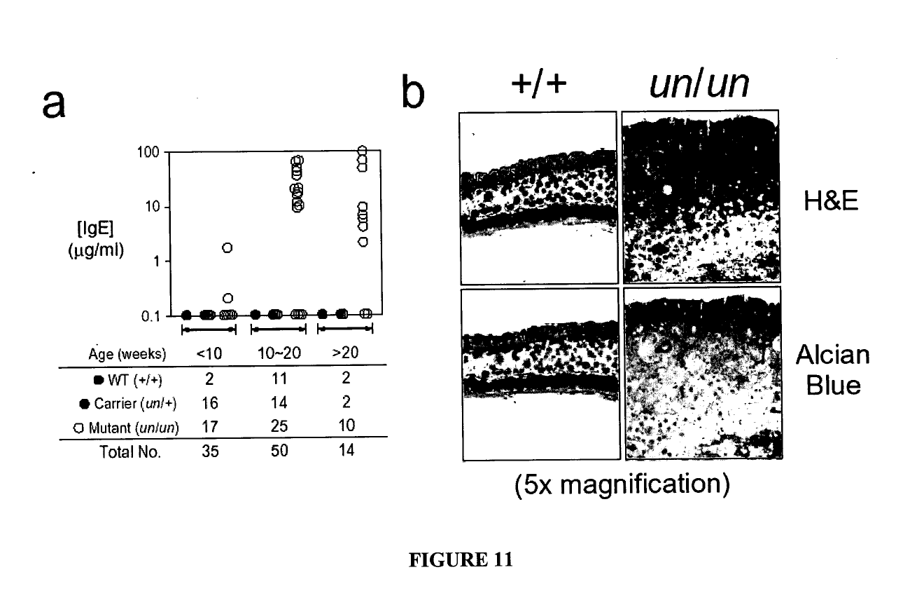

While young unmodulated mice are healthy and have normal serum Ig levels other than the reduction in serum IgM noted above, the majority of homozygous mutant mice but none of their un/+ or +/+ littermates begin to develop high serum levels of IgE, teary eyes, and erythematous ears at 10-20 weeks of age, see FIG. 11a, which illustrates serum IgE concentration in mice of different ages.

Their ears become intensely itchy, progressing to dermatitis of the ear and neck. Histologically, the inflamed regions of skin exhibit hyperkeratosis, extensive mast cell infiltrates, mononuclear cells, and occasional eosinophils, see FIG. 11b. This spontaneous atopic disorder was also observed in mice with B6xNODk F2 hybrid background as part of the mapping cross, and again was strictly correlated with homozygosity for the CARD11 (Carma-1) mutation, see FIG. 11b, which illustrates representative histology of wildtype and mutant mouse skin with atopic dermatitis.

(III) “Cork-Screw” Tail

Identification of the Mutation in “Unmodulated” Mice

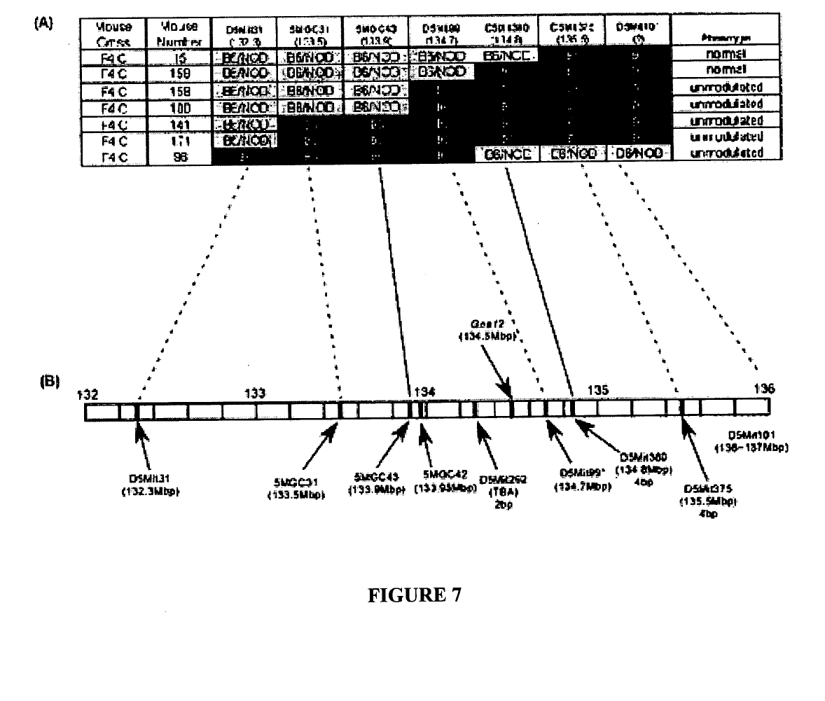

Affected animals were out-crossed to the NOD strain of mice. Offspring from this cross were interbred and the resulting mice were typed for the unmodulated phenotype. These mice were also genotyped using microsatellite markers. This approach defined a region of the mouse genome encompassing 950,000 bases. This region was predicted to contain 18 genes. See FIG. 7.

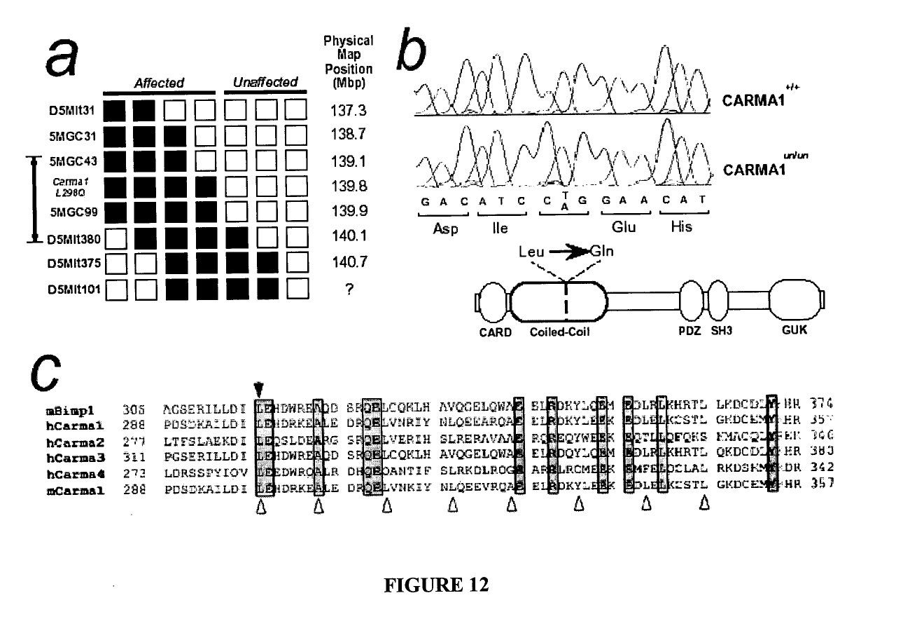

The chromosomal location of unmodulated was initially mapped to a region of chromosome 5 in an F2 intercross between un/un C57BL/6 homozygotes and the NOD.B10BR-H-2k strain. Since no known components of BCR signaling were encoded in this region, mapping via an F3 and F4 intercross was continued, localizing the defect to a 1 Mb region, see FIG. 12a. FIG. 12 shows illustrates aspects of the identification of the mutated gene in unmodulated mice. FIG. 12a illustrates high-resolution haplotype matrix representing a part of mouse chromosome 5 (|ι| symbol denotes the maximum non-recombinant interval). Numbers on the right column indicate the physical map position of SSLP markers based on public mouse genome assembly. Carmal L298Q is an allele-specific PCR marker differentiating the T to A mutation.

Two genes within this region were identified as potential candidates. One was the G-protein alpha 12 subunit, Gna12, which has been shown to interact with and activate Btk. Re-sequencing of Gna12 mRNA from un/un spleen cells and un/un genomic DNA did not reveal a mutation. The other candidate was annotated as a coiled-coil protein and was identified by BLAST and comparison with the Celera human genome database as encoding the mouse orthologue of CARMA-1/Card11/Bimp3. Re-sequencing of mRNA and exons in genomic DNA revealed a single T←A nucleotide substitution in unmodulated mice resulting in a Leu->Gln codon substitution in the encoded protein, see FIG. 12b. FIG. 12b illustrates electropherogram traces showing point-mutation and resulting codon substitution in unmodulated. Predicted amino-acid translation of nucleotide sequence adjacent point-mutation is shown in upper panel. A schematic diagram of mouse Carmal structure and location of mutation is the lower panel.

The altered leucine lies within a segment predicted by the COILS program to form a coiled-coil domain, and is a conserved leucine in the heptad repeats of the predicted alpha helical coil, see FIG. 12c. FIG. 12c illustrates partial amino-acid sequences of coiled-coil domain in mouse and human Carma proteins are aligned. Residues that are conserved in 100% of analyzed proteins are boxed and shaded. The filled arrowhead indicates mutated leucine in unmodulated. Unfilled arrowheads indicate heptad repeats of hydrophobic amino acids.

In other proteins, the repeating leucines of coiled-coil domains participate in pairing of these domains to form stable protein dimers or multimers. The change to glutamine would thus be predicted to interfere with CARD11 (Carma-1) self-association or association with other proteins, but future experiments will be needed to distinguish between this possibility and the alternative that the residue change destabilizes the protein.

To identify the mutation, these genes were sequenced from mutant unmodulated mice. This led to the identification of a mutation in the mouse homologue of the human gene CARD11. The identified CARD11 allele of the inbred strain has a single base pair residue change as compared to wild type murine CARD11. The changed, or mutant, sequence in the inbred mouse of the invention is at base residue number 907 in SEQ ID NO:1, where an ‘A’ is changed to a ‘T’ causing a change in nucleic acid subsequence “ACATCCTGGAACATGACC” to “ACATCCAGGAACATGACC” (see SEQ ID NO:4). This nucleic acid change results in a change of amino acid from leucine to glutamine at amino acid 298 of SEQ ID NO:5, and causing a change in amino acid subsequence ‘DILEH’ to ‘DIQEH’.

Re-sequencing of Gna12 mRNA from un/un spleen cells and genomic DNA did not reveal a mutation. The other candidate was annotated as a coiled-coil protein. It was identified by BLAST and comparison with the CELERA™ human genome database as encoding the mouse orthologue of CARMA-11/Card11/Bimp3. Re-sequencing of mRNA and exons in genomic DNA revealed a single T to A nucleotide substitution in unmodulated mice resulting in a Leu->Gln codon substitution in the encoded protein.

Summary of “Unmodulated” Mice

While the invention is not limited to any particular mechanism of action, the phenotype of the mouse mutant of the invention, unmodulated, defines CARD11 (Carma-1) as a selective step in immunogenic signaling by B and T cell antigen receptors. The evidence that the mutation in CARD11 (Carma-1) is responsible for the unmodulated phenotype is as follows. First, the B cell phenotype resembles that of mouse mutations in other BCR signaling molecules such as Btk, BCAP, PI3kinase, PLCg2, vav1/2, and Bcl10. Second, the defect was mapped to 1 Mb of DNA containing 18 predicted genes and the only other candidate for BCR signaling, Gna12, does not contain a mutation. Third, the defects in Bcl-10 phosphorylation and IkBa degradation are consistent with the physical interaction between CARD11 (Carma-1) and Bcl-10 and with the phenotype of T lymphoma cells lacking or overexpressing wild-type or dominant negative CARD11 (Carma-1).

In one aspect, the invention provides CARD11 polypeptides (and the nucleic acids encoding them) where one, some or all of the CARD11 heptad repeat leucines are replaces by another amino acid, e.g., substituted with a polar amino acid residue, e.g., substituted with a polar glutamine residue. In one aspect, the invention provides methods to disrupt the interaction of CARD11 (Carma-1) with other CC domain proteins, such as the B cell adaptor protein BCAP or the related molecule BANK, or methods to disrupt self-assembly of CARD11 (Carma-1) into higher order structures. Coiled-coil domains occur in diverse proteins, where their typical function is to promote formation of a two-, three-, or four-stranded bundle of interwound alpha helices. For example, this domain forms stable dimers in the leucine zipper family of transcription factors and stable tetramers in lac repressor. Since the heptad repeat leucines mediate helix-helix interactions in CC domains, substitution with a polar residue, such as a glutamine residue, can disrupt the interaction of CARD11 (Carma-1) with other CC domain proteins, such as the B cell adaptor protein BCAP or the related molecule BANK, or can disrupt self-assembly of CARD11 (Carma-1) into higher order structures.

The invention provides methods to promote immunogenic signaling in lymphocytes by, e.g., forming supramolecular signalosomes. While the invention is not limited to any particular mechanism of action, by analogy with the functions of other PDZ proteins such as INAD, PSD95 and disks large, in one aspect CARD11 (Carma-1) promotes immunogenic signaling in lymphocytes by forming supramolecular signalosomes (e.g., “immunons”) that couple the initial signaling events following antigen binding to NFkB and to sustain JNK activation. The early events in BCR signaling such as protein tyrosine phosphorylation and elevation of intracellular calcium occur normally in CARD11 (Carma-1) mutant B cells, and activation of NFAT and ERK takes place normally. These pathways represent events that are maximally triggered within 1 to 3 minutes of antigen receptor engagement in naïve B cells, and they selectively remain coupled to the BCR in anergic cells where they may help to maintain a state of tolerance.

Chronic signaling through NFAT appears to play a similar role in T cell anergy. By contrast, coupling to NFkB follows a slower tempo requiring 10-30 minutes of antigen stimulation. Similarly, phosphorylation of Bcl-10 appears slow compared to ERK or JNK activation, see FIG. 9. This tempo parallels the tempo of BCR capping with lipid rafts and cytoskeletal elements, a process that has been correlated with immunogenic signaling by BCRs. In the same way, the early events in TCR signaling precede formation of a supramolecular activation complex or synapse, but formation of this complex is correlated with activation of NFkB based on the fact that CD28, PKCtheta and CARD11 (Carma-1) are concentrated in these structures and selectively augment this connection.

The invention provides methods for co-clustering a signalosome containing key enzymes for NFkB activation, e.g., Btk, PKCtheta or PKCbeta, adaptor proteins like Bcl-10 and Malt-1, and the macromolecular IkB kinase complex responsible for activating NFkB. While the invention is not limited to any particular mechanism of action, like INAD or disks large, CARD11 (Carma-1) can co-cluster a signalosome containing key enzymes for NFkB activation such as Btk, PKCtheta or PKCbeta, adaptor proteins like Bcl-10 and Malt-1, and the macromolecular IkB kinase complex responsible for activating NFkB.

The spontaneous atopic disorder in CARD11 (Carma-1) mutant mice may have several causes. One possibility is that Th2 responses are less dependent on signaling to JNK and NFkB. This possibility is supported by the exaggerated Th2 cell formation in mice with JNK signaling defects, the severe Th2-like disorder in mice with a point mutation in LAT that reduces phospholipase C activation, and the finding that Th2 cells do not assemble supramolecular activation complexes. Similarly, Th2/atopic disorders characterize children with Wiskott-Aldrich syndrome, where a mutation in WASP interferes with the capacity of TCRs to reorganize the cytoskeleton and form concentrated supramolecular complexes. Because CARD11 (Carma-1) is expressed in a range of hemopoietic cells, in one aspect its defective expression causes defects in B cells, mast cells, or other cell types that can bring about an atopic disorder.

The invention provides methods for selective interference with NFkB and immunosuppressive therapies. The CARD11 (Carma-1) step in antigen receptor signaling make it a target for immunosuppressive therapy. Current immunosuppressive strategies based on calcineurin antagonists cyclosporin A and FK506 block antigen receptor activation of NFAT and NFkB. Thus, they may interfere with tolerogenic and immunogenic signaling. In one aspect, the selective interference methods of the invention (e.g., in one aspect, with NFkB) selectively preserve tolerogenic signaling to establish a self-reinforcing state of tolerance. While the invention is not limited to any particular mechanism of action, the phenotype of CARD11 (Carma-1) mutant mice indicates that selective inhibition of this pathway selectively antagonizes immunogenic signaling by antigen receptors.

While the invention has been described and exemplified in sufficient detail for those skilled in this art to make and use it, various alternatives, modifications, and improvements should be apparent without departing from the spirit and scope of the invention. The present invention is well adapted to carry out the objects and obtain the ends and advantages mentioned, as well as those inherent therein. The examples provided here are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

The disclosure of all publications cited above are expressly incorporated herein by reference, each in its entirety, to the same extent as if each were incorporated by reference individually.

The invention illustratively described herein suitably may be practiced in the absence of any element or elements, limitation or limitations, which is not specifically disclosed herein. Thus, for example, in each instance herein any of the terms “comprising”, “consisting essentially of” and “consisting of” maybe replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. An isolated or recombinant nucleic acid comprising a nucleic acid sequence having at least 90% sequence identity to SEQ ID NO:1, wherein the nucleic acid encodes at least one polypeptide having an NFkB activating activity.

2. The isolated or recombinant nucleic acid of claim 1, wherein the NFkB activating activity comprises mediating the assembly of CARD-containing proteins into apoptosis signaling complexes.

3. The isolated or recombinant nucleic acid of claim 1, wherein the NFkB activating activity comprises mediating the assembly of CARD-containing proteins into NF-kappaB signaling complexes.

4. The isolated or recombinant nucleic acid of claim 1, wherein the NFkB activating activity comprises being a molecular scaffold for the assembly of a multi-molecular complex.

5. The isolated or recombinant nucleic acid of claim 1, wherein the NFkB activating activity comprises recruitment of a signaling protein into a multi-molecular complex.

6. The isolated or recombinant nucleic acid of claim 5, wherein the signaling protein comprises a Bcl10, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

7. An isolated or recombinant nucleic acid comprising a nucleic acid sequence having at least 90% sequence identity to SEQ ID NO:3.

8. An isolated or recombinant nucleic acid comprising a nucleic acid sequence having at least 90% sequence identity to SEQ ID NO:4.

9. A first nucleic acid molecule for identifying a second nucleic acid molecule, wherein the second nucleic acid molecule encodes a polypeptide with an NFkB activating activity, wherein the first nucleic acid molecule comprises at least 10 consecutive bases of a sequence as set forth in SEQ ID NO:1, SEQ ID NO:3 or SEQ ID NO:4, and further wherein the first nucleic acid molecule identifies the second nucleic acid molecule by binding or hybridization.

10. An expression cassette comprising a nucleic acid comprising a nucleic acid sequence having at least 90% sequence identity to SEQ ID NO:1, SEQ ID NO:3, or SEQ ID NO:4.

11. A vector comprising a nucleic acid comprising a nucleic acid sequence having at least 90% sequence identity to SEQ ID NO:1, SEQ ID NO:3, or SEQ ID NO:4.

12. A transformed cell comprising a vector according to claim 11.

13. The transformed cell of claim 14, wherein the cell is a bacterial cell, a mammalian cell, a fungal cell, a yeast cell, an insect cell or a plant cell.

14. A cloning vehicle comprising a nucleic acid sequence having at least 90% sequence identity to SEQ ID NO:1, SEQ ID NO:3, or SEQ ID NO:4.

15. The cloning vehicle of claim 12, wherein the nucleic acid sequence is contained within a recombinant virus, a plasmid, a phage, a phagemid, a cosmid, a fosmid, a bacteriophage or an artificial chromosome.

16. An antisense oligonucleotide comprising a nucleic acid sequence complementary to or capable of hybridizing under stringent conditions to a nucleic acid sequence with at least 90% sequence identity to SEQ ID NO:1, SEQ ID NO:3, or SEQ ID NO:4.

17. A method of inhibiting an NFkB activity in a cell comprising administering to the cell or expressing in the cell an antisense oligonucleotide according to claim 16.

18. A double stranded RNA oligonucleotide comprising a nucleic acid sequence complementary to or capable of hybridizing under stringent conditions to a nucleic acid sequence with at least 90% sequence identity to SEQ ID NO:1, SEQ ID NO:3, or SEQ ID NO:4.

19. A method of inducing the degradation by RNA interference of a message in a cell comprising administering to the cell or expressing in the cell a double stranded RNA molecule, or a molecule predicted to fold into a double stranded form, or two complementary RNA molecules that are capable of hybridizing to form a double stranded RNA molecule, wherein the double stranded RNA molecule comprises a nucleic acid sequence complementary to or capable of hybridizing under stringent conditions to a nucleic acid sequence at least 90% sequence identity to SEQ ID NO:1, SEQ ID NO:3, or SEQ ID NO:4.

20. An isolated or recombinant polypeptide comprising (i) an amino acid sequence having at least 95% sequence identity to SEQ ID NO:2; or (ii) an amino acid sequence encoded by a nucleic acid comprising a sequence having at least 90% sequence identity to SEQ ID NO:1; wherein the polypeptide has an NFkB activating activity.

21. The isolated or recombinant polypeptide of claim 20, wherein the NFkB activating activity comprises mediating the assembly of CARD-containing proteins into apoptosis signaling complexes.

22. The isolated or recombinant polypeptide of claim 20, wherein the NFkB activating activity comprises mediating the assembly of CARD-containing proteins into NF-kappaB signaling complexes.

23. The isolated or recombinant polypeptide of claim 20, wherein the NFkB activating activity comprises being a molecular scaffold for the assembly of a multi-molecular complex.

24. The isolated or recombinant polypeptide of claim 20, wherein the NFkB activating activity comprises recruitment of a signaling protein into a multi-molecular complex.

25. The isolated or recombinant polypeptide of claim 24, wherein the signaling protein comprises a Bcl10, a calcineurin, a PKCtheta, a PKCbeta, an IKKalpha, an IKKbeta, an IKKgamma, an IkappaB (IκB), a vav, a MALT1, an AKT/PKB, an MEKK1, an MEKK2, an MLK3, a Cot/Tp12 or an NIK.

26. An isolated or recombinant antibody that specifically binds to a polypeptide according to claim 20.

27. A hybridoma comprising an antibody according to claim 26.

28. A method for identifying a polypeptide having an NFkB activating activity, said method comprising:
contacting a polypeptide according to claim 20, or a fragment thereof, with two or more molecules that multimerize or specifically associate in the presence of an NFkB activating polypeptide; and
detecting multimerization or specific association of the molecules;
wherein multimerization or specific association of the molecules identifies the polypeptide as having an NFkB activating activity.

29. A method for identifying a polypeptide having an NFkB activating activity, said method comprising:
contacting a polypeptide according to claim 20, or a fragment thereof, with a construct comprising an NFkB-responsive promoter operably linked to a reporter gene; and
detecting the amount of reporter gene product produced;
wherein an increase in the amount of reporter gene product identifies the polypeptide as having an NFkB activating activity.

30. A method of determining whether a test compound specifically binds to a polypeptide according to claim 20, or a fragment thereof, said method comprising:
contacting the polypeptide or fragment thereof with the test compound; and
determining whether the test compound specifically binds to the polypeptide, thereby determining that the test compound specifically binds to the polypeptide.

31. A method for identifying a modulator of an NFkB activating activity, said method comprising:
contacting a polypeptide according to claim 20, or a fragment thereof, with a test compound; and
measuring an activity of the NFkB activating polypeptide;
wherein a change in NFkB activating activity measured in the presence of the test compound as compared to the NFkB activating activity in the absence of the test compound provides a determination that the test compound modulates an activity of the NFkB activating polypeptide.

32. The method of claim 31, further comprising providing two or more molecules that multimerize or specifically associate in the presence of an NFkB activating polypeptide, wherein the NFkB activating activity is measured by detecting an increase or decrease in the amount of multimerization or specific association of the molecules.

33. A method for identifying a polypeptide able to upregulate the activity of an NFkB activity, said method comprising:
contacting a polypeptide according to claim 20, or a fragment thereof, with a reporter gene with activity determined by the activation state of an NFkB; and
detecting an increase in reporter gene activity;
wherein an increase in the amount of reporter gene activity identifies a polypeptide able to upregulate the activity of NFkB.

34. A method for determining a functional fragment of an NFkB activating polypeptide, said method comprising:
deleting a plurality of amino acid residues from a polypeptide according to claim 20 to create a subsequence thereof; and
testing the subsequence for an NFkB activating activity, thereby determining a functional fragment of an NFkB activating polypeptide.

35. A transgenic non-human animal comprising a heterologous nucleic acid, wherein the nucleic acid comprises a sequence having at least 90% sequence identity to SEQ ID NO:1 SEQ ID NO:3, or SEQ ID NO:4, wherein said animal exhibits a phenotype, relative to a wild-type phenotype comprising a characteristic selected from the group consisting of a dermatitis, a B cell defect, a T cell defect, and a combination of any two or more thereof.

36. The transgenic non-human animal of claim 35, wherein the animal is a mouse or a rat.

37. A cell or cell line derived from a transgenic non-human animal according to claim 35.

38. An in vitro method of screening for a modulator of an NFkB activating activity, said method comprising:
contacting a cell or cell line according to claim 37 with a test compound; and
detecting an increase or a decrease in the amount of an NFkB reporter gene, an NFkB transcript, an NFkB protein, or an NFkB activity; thereby identifying the test compound as a modulator of an NFkB activating activity.

39. An in vivo method of screening for a modulator of an NFkB activating activity, said method comprising:
contacting a transgenic non-human animal according to claim 35 with a test compound; and
detecting an increase or a decrease in the amount of an NFkB reporter gene, an NFkB transcript, an NFkB protein, or an NFkB activity; thereby identifying the test compound as a modulator of an NFkB activating activity.

40. An in vivo method for screening for a modulator of a dermatitis, a B cell defect, or a T cell defect, said method comprising:
contacting a transgenic non-human animal according to claim 35 with a test compound; and
detecting an increase or a decrease in the amount or severity of the dermatitis, the B cell defect, or the T cell defect;
wherein the increase or the decrease identifies the test compound as a modulator of the dermatitis, the B cell defect, or the T cell defect.

41. An in vivo method to identify a genetic modulator of a dermatitis, a B cell defect, or a T cell defect, said method comprising:
inserting a test gene into one or more cells of a transgenic non-human animal according to claim 35; and
detecting an increase or a decrease in the amount or severity of the dermatitis, the B cell defect, or the T cell defect;
wherein the increase or decrease identifies the test gene as a genetic modulator of the dermatitis, the B cell defect, or the T cell defect.

42. An in vivo method to identify a genetic modulator of a dermatitis, a B cell defect, or a T cell defect, said method comprising:
mating a first transgenic non-human animal according to claim 35 with a second non-human animal of a sex opposite of the first transgenic non-human animal, wherein the second non-human animal is selected from the group consisting of an inbred non-human animal strain, a randomly mutagenized non-human animal, a transgenic non-human animal, and a knockout non-human animal; and
selecting an offspring of the mating that exhibits an increase or a decrease in the amount or severity of the dermatitis, the B cell defect, or the T cell defect, thereby identifying a genetic modulator of the dermatitis, the B cell defect, or the T cell defect.

43. An in vivo method to identify a genetic modulator of a dermatitis, a B cell defect, or a T cell defect, said method comprising:
(i) mating a first transgenic non-human animal according to claim 35 with a second non-human animal of a sex opposite of the first transgenic non-human animal, wherein the second non-human animal is a randomly mutagenized non-human animal;
(ii) mating two offspring of the mating of step (i); and
(iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

44. An in vivo method to identify a genetic modulator of a dermatitis, a B cell defect, or a T cell defect, said method comprising:
(i) mating a first transgenic non-human animal according to claim 35 with a second non-human animal of a sex opposite of the first transgenic non-human animal, wherein the second non-human animal is a randomly mutagenized non-human animal;
(ii) mating an offspring of the mating of step (i) with a transgenic non-human animal according to claim 35; and
(iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

45. An in vivo method to identify a genetic modulator of a dermatitis, a B cell defect, or a T cell defect, said method comprising:
(i) mating a first transgenic non-human animal according to claim 35 with a second non-human animal of a sex opposite of the first transgenic non-human animal, wherein the second non-human animal is a randomly mutagenized non-human animal;
(ii) mating an offspring of the mating of step (i) with a randomly mutagenized non-human animal; and
(iii) identifying offspring of the mating of step (ii) that carry a mutated allele of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

46. A knockout non-human animal, wherein an endogenous gene sequence comprising a nucleic acid sequence having at least 90% sequence identity to SEQ ID NO:1 or SEQ ID NO:3 is disrupted so as to produce a phenotype comprising a characteristic selected from the group consisting of a dermatitis, a B cell defect, a T cell defect, and a combination of any two or more thereof.

47. The knockout non-human animal of claim 46, wherein the animal is a mouse or a rat.

48. A cell or cell line derived from a knockout non-human animal according to claim 46.

49. An in vivo method for screening for a modulator of a dermatitis, a B cell defect, or a T cell defect, said method comprising:
contacting a knockout non-human animal according to claim 46 with a test compound; and
detecting an increase or a decrease in the amount or severity of the dermatitis, the B cell defect, or the T cell defect;
wherein the increase or the decrease identifies the test compound as a modulator of the dermatitis, the B cell defect, or the T cell defect.

50. An in vivo method to identify a genetic modulator of a dermatitis, a B cell defect, or a T cell defect, said method comprising:
inserting a test gene into one or more cells of a knockout non-human animal according to claim 46; and
detecting an increase or a decrease in the amount or severity of the dermatitis, the B cell defect, or the T cell defect;
wherein the increase or decrease identifies the test gene as a genetic modulator of the dermatitis, the B cell defect, or the T cell defect.

51. An inbred mouse comprising a genome that is homozygous for a nucleic acid sequence encoding a polypeptide having at least 95% sequence identity to SEQ ID NO:2, wherein said polypeptide comprises a change in the amino acid sequence of SEQ ID NO:2 at amino acid residue number 298.

52. An inbred mouse according to claim 51, wherein the polypeptide comprises a sequence as set forth in SEQ ID NO:5.

53. An inbred mouse according to claim 51, wherein the mouse has a phenotype comprising a characteristic selected from the group consisting of a dermatitis, a B cell defect, and a T cell defect.

54. A cell or cell line derived from an inbred mouse according to claim 51.

55. An in vitro method of screening for a modulator of an NFkB activating activity, said method comprising:
contacting a cell or cell line according to claim 54 with a test compound; and
detecting an increase or a decrease in the amount of an NFkB reporter gene, an NFkB transcript, an NFkB protein, or an NFkB activity; thereby identifying the test compound as a modulator of an NFkB activating activity.

56. An in vivo method of screening for a modulator of an NFkB activating activity, said method comprising:
contacting an inbred mouse according to claim 51 with a test compound; and
detecting an increase or a decrease in the amount of an NFkB reporter gene, an NFkB transcript, an NFkB protein, or an NFkB activity; thereby identifying the test compound as a modulator of an NFkB activating activity.

57. An in vivo method for screening for a modulator of a dermatitis, a B cell defect, a T cell defect, said method comprising:
contacting an inbred mouse according to claim 51 with a test compound; and
detecting an increase or a decrease in the amount or severity of the dermatitis, the B cell defect, or the T cell defect;
wherein the increase or the decrease identifies the test compound as a modulator of the dermatitis, the B cell defect, or the T cell defect.

58. An in vivo method to identify a genetic modulator of a dermatitis, a B cell defect, or a T cell defect, said method comprising:
inserting a test gene into one or more cells of an inbred mouse according to claim 51; and
detecting an increase or a decrease in the amount or severity of the dermatitis, the B cell defect, or the T cell defect;
wherein the increase or decrease identifies the test gene as a genetic modulator of the dermatitis, the B cell defect, or the T cell defect.

59. An in vivo method to identify a genetic modulator of a dermatitis, a B cell defect, or a T cell defect, said method comprising:
mating a first inbred mouse according to claim 51 with a second mouse of a sex opposite of the first inbred mouse, wherein the second mouse is selected from the group consisting of an inbred mouse strain, a randomly mutagenized mouse, a transgenic mouse, and a knockout mouse; and
selecting an offspring of the mating that exhibits an increase or a decrease in the amount or severity of the dermatitis, the B cell defect, or the T cell defect, thereby identifying a genetic modulator of the dermatitis, the B cell defect, or the T cell defect.

60. An in vivo method to identify a genetic modulator of a dermatitis, a B cell defect, or a T cell defect, said method comprising:
(i) mating a first inbred mouse according to claim 51 with a second mouse of a sex opposite of the first inbred mouse, wherein the second mouse is a randomly mutagenized non-human animal;
(ii) mating two offspring of the mating of step (i); and
(iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

61. An in vivo method to identify a genetic modulator of a dermatitis, a B cell defect, or a T cell defect, said method comprising:
(i) mating a first inbred mouse according to claim 51 with a second mouse of a sex opposite of the first inbred mouse, wherein the second mouse is a randomly mutagenized non-human animal;
(ii) mating an offspring of the mating of step (i) with an inbred mouse according to claim 51; and
(iii) identifying offspring of the mating of step (ii) that carry two mutated alleles of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

62. An in vivo method to identify a genetic modulator of a dermatitis, a B cell defect, or a T cell defect, said method comprising:
(i) mating a first inbred mouse according to claim 51 with a second mouse of a sex opposite of the first inbred mouse, wherein the second mouse is a randomly mutagenized mouse;
(ii) mating an offspring of the mating of step (i) with a randomly mutagenized mouse; and
(iii) identifying offspring of the mating of step (ii) that carry a mutated allele of a nucleic acid having at least 90% identity with SEQ ID NO:1 and that exhibit an increase or a decrease in the amount or severity of the dermatitis, the B cell defect, or the T cell defect, thereby identifying a genetic modulator of the dermatitis, the B cell defect, or the T cell defect.

63. A method for generating a toleragenic signal in a subject, said method comprising administering to the subject an amount of an antisense oligonucleotide according to claim 16 sufficient to inhibit the expression of a CARD11 polypeptide, thereby generating a T cell defect and generating a toleragenic signal in the subject.

64. A method for generating a toleragenic signal in a subject, said method comprising administering to the subject an amount of an antibody according to claim 26 sufficient to inhibit the activity of a CARD11 polypeptide, thereby generating a T cell defect and generating a toleragenic signal in the subject.

65. A method for tolerizing a subject to an antigen, said method comprising:
administering to the subject an amount of an antisense polynucleotide according to claim 16 sufficient to inhibit the expression of a CARD11 polypeptide, thereby generating a T cell defect and generating a toleragenic signal in the subject; and
administering an antigen to the subject, thereby tolerizing the subject to the antigen.

66. A method for tolerizing a subject to an antigen, said method comprising:
administering to the subject an amount of an antibody according to claim 26 sufficient to inhibit the expression of a CARD11 polypeptide, thereby generating a T cell defect and generating a toleragenic signal in the subject; and
administering an antigen to the subject, thereby tolerizing the subject to the antigen.

67. A method for tolerizing a subject to an antigen, said method comprising:
administering to the subject an amount of small molecule inhibitor to a polypeptide having an NFkB activating activity according to claim 20 sufficient to inhibit the expression of a CARD11 polypeptide, thereby generating a T cell defect and generating a toleragenic signal in the subject; and
administering an antigen to the subject, thereby tolerizing the subject to the antigen.

68. A method of treating an autoimmune disease or a lymphoma, said method comprising modulating a polypeptide having NFkB activating activity by administering a compound that binds to or modulates a polypeptide comprising (i) an amino acid sequence having at least 95% sequence identity to SEQ ID NO:2; or (ii) an amino acid sequence encoded by a nucleic acid comprising a sequence having at least 90% sequence identity to SEQ ID NO:1; wherein the polypeptide has an NFkB activating activity.


SEQUENCE LISTING (35 sequences)

SEQ ID NO: 1 - DNA, 3494 bp, Homo sapiens
  CDS (15)..(3455)
gaggagggccagctatggatgactacatggagacgctgaaggatgaagaggaggccctatgggataacgtggaatgcaaccggcacatgctgagccgttacatcaaccccgccaagctcaccccctacctgcgccagtgcaaggtcatcgatgagcaagatgaagacgaggtgctcaatgcgcccatgctgccgtccaagatcaaccgtgcaggccgattgttggacattcttcacaccaagggacaaaggggctatgtggtcttcctggagagcctggagttttactacccagaactttacaaactggtgactggaaaggaacccacccggagattctccaccattgtggtggaggaaggccatgagggcctcacacacttcctgatgaacgaggtcatcaaactgcagcagcaagtgaaagccaaggaccttcagcgctgtgagctgctggccaagtcccggcaactggaggatgagaagaagcagctgagcctgatacgggtggagctgctgaccttccaggagcgatactacaagatgaaggaggagcgggacagctacaatgacgagctcgtcaaggtcaaggacgacaactacaacttagccatgcgctacgcccagctcagtgaggagaaaaacatggcggtgatgaggagccgcgacctccaactcgagatcgaccagctcaaacaccgactgaacaagatggaggaggaatgcaagctggagagaaatcagtccctcaagctcaagaatgacatcgagaaccggcccaggaaggagcaggtcctggagctggagcgggagaatgagatgctgaagacgaaaattcaggagctgcagtccatcatccaggctggcaagcgcagcctccctgactcagacaaggccatcttggacatcctggaacatgaccggaaggaggcgctagaggaccggcaggaactggtcaacaaaatttacaacctacaagaggaagtccgccaggcggaggagctgcgggataagtacctggaggagaaggaagacctggaactcaagtgttcaaccctggggaaggactgtgaaatgtacaagcaccgcatgaacacagttatgctgcagctggaggaggtggagcgggagcgggaccaggccttccactcccgagatgaggcacagacacagtactcacagtgcttaatcgagaaggacaagtaccggaagcagatccgggagctggaggagaagaacgatgagatgcgtattgagatggtgaggagggaggcctgtattgtcaacctggaaagcaagctccggcgcctgtccaaggacaacggcagcctcgaccagagtctgcctagacaccttccagccaccatcatctcacagaaccttggagacaccagccccaggaccaatggccaggaagctgatgattcttcaacctcagaagagtctcccgaagacagcaagtactttctgccttaccacccaccccggcgccggatgaacctaaagggcatccagctgcagagagccaaatcccccatcagcatgaagcaagcatctgagtttcaagtcaaggggcacgaagaggatttcacagacggcagccccagttcctcccgctcgctgcctgtcaccagctctttctccaagatgcaaccccatcggagccgcagcagcatcatgtcaatcacggcagagcccccgggaaatgactccatagtcagacgctgtaaggaagatgcgccacaccggagcacggtggaagaagacaacgatagctgtgggtttgatgccttagaccttgacgatgaaaatcacgaacgttattcctttggacctccctccatccactcctcctcctcttcacaccagtcagagggactggatgcctacgacctggagcaggtcaacctcatgttacgaaagttctctttggaaaggcccttccggccatcggtcacatctgggggtcacgtgcggggcaccgggcccttggtccagcacacaactctgaatggcgatgggctcatcacgcagctcacccttctgggcggcaatgcacgcgggagcttcattcactctgtcaagccaggctcactggctgagagggccggactgcgtgagggccaccaactcctgctgctggaaggttgcatccgaggcgaaaggcagagcgttccactggatgcgtgcacaaaagaagaggcccgttggaccatccagaggtgcagtggcctcatcactctgcattacaaggtcaaccatgaaggataccggaagctgctgaaggagatggaggatggtctgatcacatcaggggactcgttctatatccgcctgaacctgaacatctccagccagctggatgcctgctccatgtccctcaagtgtgacgacgtggtgcatgtcctagacaccatgtaccaggacaggcacgagtggctgtgtgcacgagtcgaccccttcactgaccaagacctggacacgggcaccatccccagctacagccgggctcaacagcttctcctggtgaagctccagcggttggttcacagaggcaaccgggaagaggcagacagcgctcaccacaccctgcgcagcctccggaacaccctgcagcccgaagagatgctttcgacgagcgacccccgagtcagcccccgcctctccagagcgagtttcttctttggccagctcctgcagtttgtcagccggtcagaaaacaagtacaaaagaatgaacagcaatgagcgcgtgagaatcatctctgggagtcccctggggagcctctcccggtcctcgctggatgccaccaaactcctgaccgagaagcatgaagaactggatcctgagaatgagctcagccggaacctcaccctgatcccttacagcctggtgcgcgctttccactgtgagcgccgcaggcctgtgctcttcacgcccaccatgctggccaagacattggtgcagaagctgctcaactcagggggtgccatggagttcaccatctgcaagtcagatattgtcacaagagatgagttcctccgaaagcagaagacagagaccatcatctactcccgggaaaagaaccccaacacctttgaatgcatcgtccctgccaacattgaggctgtggcagccaagaacaaacactgcctgctggaggctgggatcggctgtgtgcgcgacctgatcaagtgcaaggtgtaccccatagtgctgctcatccgggtgagcgagaagaacatcaaacggttcaggaagctgctgccgcggccagagacggaagaggaattcctgcgagtgtgcaggctcaaagagaaggagctggaggcgctgccctgcctctacgccaccgtggaagctgagatgtggagcagcgtggaggagctgctgcgagtcctcaaagacaagattgtagaggagcagcgcaagaccatctgggtggacgaggaccagctgtgagcttgtctgggtctgacctacacacagacacaccgg

SEQ ID NO: 2 - protein, 1147 aa, Homo sapiens
MDDYMETLKDEEEALWDNVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQVKAKDLQRCELLAKSRQLEDEKKQLSLIRVELLTFQERYYKMKEERDSYNDELVKVKDDNYNLAMRYAQLSEEKNMAVMRSRDLQLEIDQLKHRLNKMEEECKLERNQSLKLKNDIENRPRKEQVLELERENEMLKTKIQELQSIIQAGKRSLPDSDKAILDILEHDRKEALEDRQELVNKIYNLQEEVRQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDNGSLDQSLPRHLPATIISQNLGDTSPRTNGQEADDSSTSEESPEDSKYFLPYHPPRRRMNLKGIQLQRAKSPISMKQASEFQVKGHEEDFTDGSPSSSRSLPVTSSFSKMQPHRSRSSIMSITAEPPGNDSIVRRCKEDAPHRSTVEEDNDSCGFDALDLDDENHERYSFGPPSIHSSSSSHQSEGLDAYDLEQVNLMLRKFSLERPFRPSVTSGGHVRGTGPLVQHTTLNGDGLITQLTLLGGNARGSFIHSVKPGSLAERAGLREGHQLLLLEGCIRGERQSVPLDACTKEEARWTIQRCSGLITLHYKVNHEGYRKLLKEMEDGLITSGDSFYIRLNLNISSQLDACSMSLKCDDVVHVLDTMYQDRHEWLCARVDPFTDQDLDTGTIPSYSRAQQLLLVKLQRLVHRGNREEADSAHHTLRSLRNTLQPEEMLSTSDPRVSPRLSRASFFFGQLLQFVSRSENKYKRMNSNERVRIISGSPLGSLSRSSLDATKLLTEKHEELDPENELSRNLTLIPYSLVRAFHCERRRPVLFTPTMLAKTLVQKLLNSGGAMEFTICKSDIVTRDEFLRKQKTETIIYSREKNPNTFECIVPANIEAVAAKNKHCLLEAGIGCVRDLIKCKVYPIVLLIRVSEKNIKRFRKLLPRPETEEEFLRVCRLKEKELEALPCLYATVEAEMWSSVEELLRVLKDKIVEEQRKTIWVDEDQL

SEQ ID NO: 3 - DNA, 313 bp, Homo sapiens
tgtttcatagaggcttttttattaaccttgttggggtttatgcctcgcaggaggagggccagctatggatgactacatggagacgctgaaggatgaagaggaggccctatgggataacgtggaatgcaaccggcacatgctgagccgttacatcaaccccgccaagctcaccccctacctgcgccagtgcaaggtcatcgatgagcaagatgaagacgaggtgctcaatgcgcccatgctgccgtccaagatcaaccgtgcaggtagtggagggaagggaagtgtgtgaaccacacatgctcagtcaagggca

SEQ ID NO: 4 - DNA, 3494 bp, Homo sapiens
  CDS (15)..(3455)
gaggagggccagctatggatgactacatggagacgctgaaggatgaagaggaggccctatgggataacgtggaatgcaaccggcacatgctgagccgttacatcaaccccgccaagctcaccccctacctgcgccagtgcaaggtcatcgatgagcaagatgaagacgaggtgctcaatgcgcccatgctgccgtccaagatcaaccgtgcaggccgattgttggacattcttcacaccaagggacaaaggggctatgtggtcttcctggagagcctggagttttactacccagaactttacaaactggtgactggaaaggaacccacccggagattctccaccattgtggtggaggaaggccatgagggcctcacacacttcctgatgaacgaggtcatcaaactgcagcagcaagtgaaagccaaggaccttcagcgctgtgagctgctggccaagtcccggcaactggaggatgagaagaagcagctgagcctgatacgggtggagctgctgaccttccaggagcgatactacaagatgaaggaggagcgggacagctacaatgacgagctcgtcaaggtcaaggacgacaactacaacttagccatgcgctacgcccagctcagtgaggagaaaaacatggcggtgatgaggagccgcgacctccaactcgagatcgaccagctcaaacaccgactgaacaagatggaggaggaatgcaagctggagagaaatcagtccctcaagctcaagaatgacatcgagaaccggcccaggaaggagcaggtcctggagctggagcgggagaatgagatgctgaagacgaaaattcaggagctgcagtccatcatccaggctggcaagcgcagcctccctgactcagacaaggccatcttggacatccaggaacatgaccggaaggaggcgctagaggaccggcaggaactggtcaacaaaatttacaacctacaagaggaagtccgccaggcggaggagctgcgggataagtacctggaggagaaggaagacctggaactcaagtgttcaaccctggggaaggactgtgaaatgtacaagcaccgcatgaacacagttatgctgcagctggaggaggtggagcgggagcgggaccaggccttccactcccgagatgaggcacagacacagtactcacagtgcttaatcgagaaggacaagtaccggaagcagatccgggagctggaggagaagaacgatgagatgcgtattgagatggtgaggagggaggcctgtattgtcaacctggaaagcaagctccggcgcctgtccaaggacaacggcagcctcgaccagagtctgcctagacaccttccagccaccatcatctcacagaaccttggagacaccagccccaggaccaatggccaggaagctgatgattcttcaacctcagaagagtctcccgaagacagcaagtactttctgccttaccacccaccccggcgccggatgaacctaaagggcatccagctgcagagagccaaatcccccatcagcatgaagcaagcatctgagtttcaagtcaaggggcacgaagaggatttcacagacggcagccccagttcctcccgctcgctgcctgtcaccagctctttctccaagatgcaaccccatcggagccgcagcagcatcatgtcaatcacggcagagcccccgggaaatgactccatagtcagacgctgtaaggaagatgcgccacaccggagcacggtggaagaagacaacgatagctgtgggtttgatgccttagaccttgacgatgaaaatcacgaacgttattcctttggacctccctccatccactcctcctcctcttcacaccagtcagagggactggatgcctacgacctggagcaggtcaacctcatgttacgaaagttctctttggaaaggcccttccggccatcggtcacatctgggggtcacgtgcggggcaccgggcccttggtccagcacacaactctgaatggcgatgggctcatcacgcagctcacccttctgggcggcaatgcacgcgggagcttcattcactctgtcaagccaggctcactggctgagagggccggactgcgtgagggccaccaactcctgctgctggaaggttgcatccgaggcgaaaggcagagcgttccactggatgcgtgcacaaaagaagaggcccgttggaccatccagaggtgcagtggcctcatcactctgcattacaaggtcaaccatgaaggataccggaagctgctgaaggagatggaggatggtctgatcacatcaggggactcgttctatatccgcctgaacctgaacatctccagccagctggatgcctgctccatgtccctcaagtgtgacgacgtggtgcatgtcctagacaccatgtaccaggacaggcacgagtggctgtgtgcacgagtcgaccccttcactgaccaagacctggacacgggcaccatccccagctacagccgggctcaacagcttctcctggtgaagctccagcggttggttcacagaggcaaccgggaagaggcagacagcgctcaccacaccctgcgcagcctccggaacaccctgcagcccgaagagatgctttcgacgagcgacccccgagtcagcccccgcctctccagagcgagtttcttctttggccagctcctgcagtttgtcagccggtcagaaaacaagtacaaaagaatgaacagcaatgagcgcgtgagaatcatctctgggagtcccctggggagcctctcccggtcctcgctggatgccaccaaactcctgaccgagaagcatgaagaactggatcctgagaatgagctcagccggaacctcaccctgatcccttacagcctggtgcgcgctttccactgtgagcgccgcaggcctgtgctcttcacgcccaccatgctggccaagacattggtgcagaagctgctcaactcagggggtgccatggagttcaccatctgcaagtcagatattgtcacaagagatgagttcctccgaaagcagaagacagagaccatcatctactcccgggaaaagaaccccaacacctttgaatgcatcgtccctgccaacattgaggctgtggcagccaagaacaaacactgcctgctggaggctgggatcggctgtgtgcgcgacctgatcaagtgcaaggtgtaccccatagtgctgctcatccgggtgagcgagaagaacatcaaacggttcaggaagctgctgccgcggccagagacggaagaggaattcctgcgagtgtgcaggctcaaagagaaggagctggaggcgctgccctgcctctacgccaccgtggaagctgagatgtggagcagcgtggaggagctgctgcgagtcctcaaagacaagattgtagaggagcagcgcaagaccatctgggtggacgaggaccagctgtgagcttgtctgggtctgacctacacacagacacaccgg

SEQ ID NO: 5 - protein, 1147 aa, Homo sapiens
MDDYMETLKDEEEALWDNVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQVKAKDLQRCELLAKSRQLEDEKKQLSLIRVELLTFQERYYKMKEERDSYNDELVKVKDDNYNLAMRYAQLSEEKNMAVMRSRDLQLEIDQLKHRLNKMEEECKLERNQSLKLKNDIENRPRKEQVLELERENEMLKTKIQELQSIIQAGKRSLPDSDKAILDIQEHDRKEALEDRQELVNKIYNLQEEVRQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDNGSLDQSLPRHLPATIISQNLGDTSPRTNGQEADDSSTSEESPEDSKYFLPYHPPRRRMNLKGIQLQRAKSPISMKQASEFQVKGHEEDFTDGSPSSSRSLPVTSSFSKMQPHRSRSSIMSITAEPPGNDSIVRRCKEDAPHRSTVEEDNDSCGFDALDLDDENHERYSFGPPSIHSSSSSHQSEGLDAYDLEQVNLMLRKFSLERPFRPSVTSGGHVRGTGPLVQHTTLNGDGLITQLTLLGGNARGSFIHSVKPGSLAERAGLREGHQLLLLEGCIRGERQSVPLDACTKEEARWTIQRCSGLITLHYKVNHEGYRKLLKEMEDGLITSGDSFYIRLNLNISSQLDACSMSLKCDDVVHVLDTMYQDRHEWLCARVDPFTDQDLDTGTIPSYSRAQQLLLVKLQRLVHRGNREEADSAHHTLRSLRNTLQPEEMLSTSDPRVSPRLSRASFFFGQLLQFVSRSENKYKRMNSNERVRIISGSPLGSLSRSSLDATKLLTEKHEELDPENELSRNLTLIPYSLVRAFHCERRRPVLFTPTMLAKTLVQKLLNSGGAMEFTICKSDIVTRDEFLRKQKTETIIYSREKNPNTFECIVPANIEAVAAKNKHCLLEAGIGCVRDLIKCKVYPIVLLIRVSEKNIKRFRKLLPRPETEEEFLRVCRLKEKELEALPCLYATVEAEMWSSVEELLRVLKDKIVEEQRKTIWVDEDQL

SEQ ID NO: 6 - DNA, 15 bp, Homo sapiens
  CDS (1)..(15)
gacatccwggaacat

SEQ ID NO: 7 - protein, 5 aa, Homo sapiens
  MOD_RES (3): Leu or Gln
DIXEH

SEQ ID NO: 8 - protein, 70 aa, Mus sp.
AGSERILLDILEHDWREAQDSRQELCQKLHAVQGELQWAEELRDKYLQEMEDLRLKHRTLLKDCDLYKHR

SEQ ID NO: 9 - protein, 70 aa, Homo sapiens
PDSDKAILDILEHDRKEALEDRQELVNRIYNLQEEARQAEELRDKYLEEKEDLELKCSTLGKDCEMYKHR

SEQ ID NO: 10 - protein, 70 aa, Homo sapiens
LTFSLAEKDILEQSLDEARGSRQELVERIHSLRERAVAAERQREQYWEEKEQTLLQFQKSKMACQLYREK

SEQ ID NO: 11 - protein, 70 aa, Homo sapiens
PGSERILLDILEHDWREAQDSRQELCQKLHAVQGELQWAEELRDQYLQEMEDLRLKHRTLQKDCDLYKHR

SEQ ID NO: 12 - protein, 70 aa, Homo sapiens
LDRSSPYIQVLEEDWRQALRDHQEQANTIFSLRKDLRQGEARRLRCMEEKEMFELQCLALRKDSKMYKDR

SEQ ID NO: 13 - protein, 70 aa, Mus sp.
PDSDKAILDILEHDRKEALEDRQELVNKIYNLQEEVRQAEELRDKYLEEKEDLELKCSTLGKDCEMYKHR

SEQ ID NO: 14 - DNA, 239 bp, Homo sapiens
tgtcatccaatcaaacacactctcaccatctggatggaacttctccccaggccgattgttggacattcttcacaccaagggacaaaggggctatgtggtcttcctggagagcctggagttttactacccagaactttacaaactggtgactggaaaggaacccacccggagattctccaccattgtgggtaagtggctttgctaccaggggcaagggaaccctagtagaaggatgtgtg

SEQ ID NO: 15 - DNA, 427 bp, Homo sapiens
ctccgccactgccccacacctcaggtctgactgggtacccctctccacagtggaggaaggccatgagggcctcacacacttcctgatgaacgaggtcatcaaactgcagcagcaagtgaaagccaaggaccttcagcgctgtgagctgctggccaagtcccggcaactggaggatgagaagaagcagctgagcctgatacgggtggagctgctgaccttccaggagcgatactacaagatgaaggaggagcgggacagctacaatgacgagctcgtcaaggtcaaggacgacaactacaacttagccatgcgctacgcccagctcagtgaggagaaaaacatggcggtgatgaggagccgcgacctccaactcgaggtggggatgcctgggctccggctgaactgagggaagggaaaagaaatgtct

SEQ ID NO: 16 - DNA, 281 bp, Mus sp.
gcattccttgacaaccatcctcacccctctgctggcgtgcttgtcttcagatcgaccagctcaaacaccgactgaacaagatggaggaggaatgcaagctggagagaaatcagtccctcaagctcaagaatgacatcgagaaccggcccaggaaggagcaggtcctggagctggagcgggagaatgagatgctgaagacgaaaattcaggagctgcagtccatcatccaggtgagacgcaccacccttgtataggggagggctaggcgggacaaggtgggt

SEQ ID NO: 17 - DNA, 254 bp, Homo sapiens
tagctcctttgtcttcctcctgtactgatctctgctcctaaccccaccaggctggcaagcgcagcctccctgactcagacaaggccatcttggacatcctggaacatgaccggaaggaggcgctagaggaccggcaggaactggtcaacaaaatttacaacctacaagaggaagtccgccaggcggaggagctgcgggataaggtgggagtactatgggtcaggagagcagcagccagccagtgcctttaacag

SEQ ID NO: 18 - DNA, 226 bp, Homo sapiens
cccctggaagacccaggatctcattgctctctcgcattgctctctcgcagtacctggaggagaaggaagacctggaactcaagtgttcaaccctggggaaggactgtgaaatgtacaagcaccgcatgaacacagttatgctgcagctggaggaggtggagcgggagcgggaccaggtacggtgccaccctggacgtggcagaccgtgaggatgcccagcagcgct

SEQ ID NO: 19 - DNA, 298 bp, Homo sapiens
ctactttactttagtttgctttgggtgaacccgtagctcttccttggcaggccttccactcccgagatgaggcacagacacagtactcacagtgcttaatcgagaaggacaagtaccggaagcagatccgggagctggaggagaagaacgatgagatgcgtattgagatggtgaggagggaggcctgtattgtcaacctggaaagcaagctccggcgcctgtccaaggacaacggcagcctcgaccaggtaggcttagctagccatgtccccatacccatggccaggtgtcccccaat

SEQ ID NO: 20 - DNA, 185 bp, Homo sapiens
agtctgcctagacaccttccagccaccatcatctcacagaaccttggagacaccagccccaggaccaatggccaggaagctgatgattcttcaacctcagaagagtctcccgaagacagcaagtactttctgccttaccacccaccccggcgccggatgaacctaaagggcatccagctgcagag

SEQ ID NO: 21 - DNA, 145 bp, Homo sapiens
ttgtgtcttctgtcttgtgtcttcccctcctctctctttcagctgcagagagccaaatcccccatcagcatgaagcaagcatctgagtttcaaggttagtaggctgcccagatcctttttgcccttgctcatctgtcaccctccc

SEQ ID NO: 22 - DNA, 183 bp, Homo sapiens
cccttttctctctcctctgttttctgggcccgcggctctgatgaggacagtcaaggggcacgaagaggatttcacagacggcagccccagttcctcccgctcgctgcctgtcaccagctctttctccaagatggtgagctgccgtaatttccactccacttacacaccttcagagtccccggg

SEQ ID NO: 23 - DNA, 202 bp, Homo sapiens
cgaggcatcgcagataaatatccggtaacacagctgtgccgcattcacagcaaccccatcggagccgcagcagcatcatgtcaatcacggcagagcccccgggaaatgactccatagtcagacgctgtaaggaagatgcgccacaccggaggtgagtgagtggcagctggaggccttggctaggtgactgaccctgtctcca

SEQ ID NO: 24 - DNA, 153 bp, Homo sapiens
cgccagtgtcgtctcaccgccccccctccccccgggcttgtttcttacagcacggtggaagaagacaacgatagctgtgggtttgatgccttagaccttgacggtatgtatgtatctgccaggcccgaaggaaccccggagggcagggtctgc

SEQ ID NO: 25 - DNA, 233 bp, Homo sapiens
ccccccccccccagtgctccaggtcctgacagaccctcattcctctgcagatgaaaatcacgaacgttattcctttggacctccctccatccactcctcctcctcttcacaccagtcagagggactggatgcctacgacctggagcaggtcaacctcatgttacgaaagttctctttggaaaggtatggaggcagggctggggagatgactctgtgggtgtagcacttgccac

SEQ ID NO: 26 - DNA, 302 bp, Homo sapiens
ttcagctcttctcttttgagtccaactgcctattttatttcccacctcaggcccttccggccatcggtcacatctgggggtcacgtgcggggcaccgggcccttggtccagcacacaactctgaatggcgatgggctcatcacgcagctcacccttctgggcggcaatgcacgcgggagcttcattcactctgtcaagccaggctcactggctgagagggccggactgcgtgagggccaccaactcctgctggtgagacatagagggagaagctacgggttgtccccaggccctccacttct

SEQ ID NO: 27 - DNA, 228 bp, Homo sapiens
tgcccatcagcagagccaaagaaaatgaacctcttgttcttgtcctgtagctggaaggttgcatccgaggcgaaaggcagagcgttccactggatgcgtgcacaaaagaagaggcccgttggaccatccagaggtgcagtggcctcatcactctgcattacaaggtcaaccatgaaggtaaacctgggccggacctggtccacacaagggtagggtacaggacgcata

SEQ ID NO: 28 - DNA, 342 bp, Homo sapiens
agacgcggtatctccagggtgcttacatttctggattacggtctcctcaggataccggaagctgctgaaggagatggaggatggtctgatcacatcaggggactcgttctatatccgcctgaacctgaacatctccagccagctggatgcctgctccatgtccctcaagtgtgacgacgtggtgcatgtcctagacaccatgtaccaggacaggcacgagtggctgtgtgcacgagtcgaccccttcactgaccaagacctggacacgggcaccatccccagctacagccggtgagtgggggatgggctcccacaccaccccaggccagcagctccctcagc

SEQ ID NO: 29 - DNA, 197 bp, Homo sapiens
acaccttgcccagtcatagctctgaggttgctcccatgtcccctccacagggctcaacagcttctcctggtgaagctccagcggttggttcacagaggcaaccgggaagaggcagacagcgctcaccacaccctgcgcagcctccgggtaggtacacaaagacacacacacacacagcccaggccctgctgccacca

SEQ ID NO: 30 - DNA, 196 bp, Homo sapiens
tactcgggagccctttcctcagcctgagctgattgacaaactcattccagaacaccctgcagcccgaagagatgctttcgacgagcgacccccgagtcagcccccgcctctccagagcgagtttcttctttggccagctcctgcaggtaaggttggtgatcggatgcccactgacttttctggcacagtgtgctgg

SEQ ID NO: 31 - DNA, 236 bp, Homo sapiens
cctgcccccaccctctaccccaccctcctaccactgtctcttctccacagtttgtcagccggtcagaaaacaagtacaaaagaatgaacagcaatgagcgcgtgagaatcatctctgggagtcccctggggagcctctcccggtcctcgctggatgccaccaaactcctgaccgagaagcatgaaggtgtgtgacgacctcgaggccccaccccacagcccagcaggggcatgtct

SEQ ID NO: 32 - DNA, 280 bp, Homo sapiens
tgccctcgggcagggcaggctctaacagcccactgtttctgttctgttagaactggatcctgagaatgagctcagccggaacctcaccctgatcccttacagcctggtgcgcgctttccactgtgagcgccgcaggcctgtgctcttcacgcccaccatgctggccaagacattggtgcagaagctgctcaactcagggggtgccatggagttcaccatctgcaagtcaggtgagcatggccaggtgacagacagagggacacaggcttcggcagcccat

SEQ ID NO: 33 - DNA, 225 bp, Homo sapiens
ccaccacaaatagcacttgccaccctgggcactaactcccctctcttcagatattgtcacaagagatgagttcctccgaaagcagaagacagagaccatcatctactcccgggaaaagaaccccaacacctttgaatgcatcgtccctgccaacattgaggctgtggcagccaaggtgagagaccctgggtactggccagactaaggccctacccaaccatctct

SEQ ID NO: 34 - DNA, 216 bp, Homo sapiens
aaggacagctgggtgggcaccactgctgatggtggctgttatacttgcagaacaaacactgcctgctggaggctgggatcggctgtgtgcgcgacctgatcaagtgcaaggtgtaccccatagtgctgctcatccgggtgagcgagaagaacatcaaacggttcaggtaaggacacccagtcctcacacctgccacacacacaccacccttactca

SEQ ID NO: 35 - DNA, 291 bp, Homo sapiens
catgtcttcccctgcctgctgtctctccctgcccacctctccacccccaggaagctgctgccgcggccagagacggaagaggaattcctgcgagtgtgcaggctcaaagagaaggagctggaggcgctgccctgcctctacgccaccgtggaagctgagatgtggagcagcgtggaggagctgctgcgagtcctcaaagacaagattgtagaggagcagcgcaagaccatctgggtggacgaggaccagctgtgagcttgtctgggtctgacctacacacagacacaccgg